SEQUENCE LISTING (20 sequences)

SEQ ID NO: 1 - protein, 110 aa, Mus musculus
MSMTDVLSAEDIKKAIGAFAAADSFDHKKFFQMVGLKKKNPDEVKKVFHILDKDKSGFIEEDELGSILKGFSSDARDLSAKETKTLLAAGDKDGDGKIGVEEFSTLVAES

SEQ ID NO: 2 - protein, 116 aa, Mus musculus
MLSCRLQCALAALCIVLALGGVTGAPSDPRLRQFLQKSLAAATGKQELAKYFLAELLSEPNQTENDALEPEDLPQAAEQDEMRLELQRSANSNPAMAPRERKAGCKNFFWKTFTSC

SEQ ID NO: 3 - protein, 466 aa, Mus musculus
MGRKKIQITRIMDERNRQVTFTKRKFGLMKKAYELSVLCDCEIALIIFNSTNKLFQYASTDMDKVLLKYTEYNEPHESRTNSDIVETLRKKGLNGCDSPDPDADDSVGHSPESEDKYRKINEDIDLMISRQRLCAVPPPSFEMPVTIPVSSHNSLVYSNPVSTLGNPNLLPLAHPSLQRNSMSPGVTHRPPSAGNTGGLMGGDLTSGAGTSAGNGYGNPRNSPGLLVSPGNLNKNIQAKSPPPMNLGMNNRKPDLRVLIPPGSKNTMPSVNQRINNSQSAQSLATPVVSVATPTLPGQGMGGYPSAISTTYGTEYSLSSADLSSLSGFNTASALHLGSVTGWQQQHLHNMPPSALSQLGACTSTHLSQSSNLSLPSTQSLSIKSEPVSPPRDRTTTPSRYPQHTTRHEAGRSPVDSLSSCSSSYDGSDREDHRNEFHSPIGLTRPSPDERESPSVKRMRLSEGWAT

SEQ ID NO: 4 - protein, 585 aa, Mus musculus
MASPGSGFWSFGSEDGSADPENPGTARAWCQVAQKFTGGIGNKLCALLYGDSGKPAEGGGSVTSRAATGKVACTCDQKPCNCPKGDVNYAFLHATDLLPACDGERPTLAFLQDVMNILLQYVVKSFDRSTKVIDFHYPNELLQEYNWELADQPQNLEEILTHCQTTLKYAIKTGHPRYFNQLSTGLDMVGLAADWLTSTANTNMFTYEIAPVFVLLEYVTLKKMREIIGWPGGSGDGIFSPGGAISNMYAMLIARYKMFPEVKEKGMAAVPRLIAFTSEHSHFSLKKGAAALGIGTDSVILIKCDERGKMIPSDLERRILEVKQKGFVPFLVSATAGTTVYGAFDPLLAVADICKKYKIWMHVDAAWGGGLLMSRKHKWKLSGVERANSVTWNPHKMMGVPLQCSALLVREEGLMQSCNQMHASYLFQQDKHYDLSYDTGDKALQCGRHVDVFKLWLMWRAKGTTGFEAHIDKCLELAEYLYTIIKNREGYEMVFDGKPQHTNVCFWFVPPSLRTLEDNEERMSRLSKVAPVIKARMMEYGTTMVSYQPLGDKVNFFRMVISNPAATHQDIDFLIEEIERLGQDL

SEQ ID NO: 5 - protein, 146 aa, Mus musculus
MTGVFDRRVPSIRSGDFQAPFPTSAAMHHPSQESPTLPESSATDSDYYSPAGAAPHGYCSPTSASYGKALNPYQYQYHGVNGSAAGYPAKAYADYGYASPYHQYGGAYNRVPSATSQPAFSWPLYREGFRRLSTSPCQNARSWPPL

SEQ ID NO: 6 - protein, 297 aa, Mus musculus
MMTMTTMADGLEGQDSSKSAFMEFGQQQQQQQQQQQQQQQQQQQQQQPPPPPPPPPPQPHSQQTSPAMAGAHYPLHCLHSAAAAAAAAGSHHHHHQHHHHGSPYASSGGNSYNHRSLAAYPYMSHSQHSPYLQSYHNSSAAAQTRGDDTDQQKTTVIENGEIRFNGKGKKIRKPRTIYSSLQLQALNHRFQQTQYLALPERAELAASLGLTQTQVKIWFQNKRSKFKKLLKQGSNPHESDPLPGSAALSPRSPALPPVWDVSASAKGVSMPPNSYMPGYSHWYSSPHQDTMQRPQMM

SEQ ID NO: 7 - protein, 372 aa, Mus musculus
MSMSPKHTTPFSVSDILSPLEESYKKVGMEGGGLGAPLAAYRQGQAAPPAAAMQQHAVGHHGAVTAAYHMTAAGVPQLSHSAVGGYCNGNLGNMSELPPYQDTMRNSASGPGWYGANPDPRFPAISRFMGPASGMNMSGMGGLGSLGDVSKNMAPLPSAPRRKRRVLFSQAQVYELERRFKQQKYLSAPEREHLASMIHLTPTQVKIWFQNHRYKMKRQAKDKAAQQQLQQDSGGGGGGGGGAGCPQQQQAQQQSPRRVAVPVLVKDGKPCQAGAPAPGAASLQSHAQQQAQQQAQAAQAAAAAISVGSGGAGLGAHPGHQPGSAGQSPDLAHHAASPAGLQGQVSSLSHLNSSGSDYGAMSCSTLLYGRTW

SEQ ID NO: 8 - protein, 2549 aa, Mus musculus
MLGTGPAVATASAATSSNVSVLQQFASGLKSRNEETRAKAAKELQHYVTMELREMSQEESTRFYDQLNHHIFELVSSSDANERKGGILAIASLIGVEGGNSTRIGRFANYLRNLLPSSDPVVMEMASKAIGRLAMAGDTFTAEYVEFEVKRALEWLGADRNEGRRHAAVLVLRELAISVPTFFFQQVQPFFDNIFVAVWDPKQAIREGAVAALRACLILTTQREPKEMQKPQWYRHTFEEAEKGFDETLAKEKGMNRDDRIHGALLILNELVRISSMEGERLREEMEEITQQQLVHDKYCKDLMGFGTKPRHITPFTSFQAVQPQQPNALVGLLGYSSPQGLMGFGTSPSPAKSTLVESRCCRDLMEEKFDQVCQWVLKCRSSKNSLIQMTILNLLPRLAAFRPSAFTDTQYLQDTMNHVLSCVKKEKERTAAFQALGLLSVAVRSEFKVYLPRVLDIIRAALPPKDFAHKRQKTVQVDATVFTCISMLARAMGPGIQQDIKELLEPMLAVGLSPALTAVLYDLSRQIPQLKKDIQDGLLKMLSLVLMHKPLRHPGMPKGLAHQLASPGLTTLPEASDVASITLALRTLGSFEFEGHSLTQFVRHCADHFLNSEHKEIRMEAARTCSRLLTPSIHLISGHAHVVSQTAVQVVADVLSKLLVVGITDPDPDIRYCVLASLDERFDAHLAQAENLQALFVALNDQVFEIRELAICTVGRLSSMNPAFVMPFLRKMLIQILTELEHSGIGRIKEQSARMLGHLVSNAPRLIRPYMEPILKALILKLKDPDPDPNPGVINNVLATIGELAQVSGLEMRKWVDELFIIIMDMLQDSSLLAKRQVALWTLGQLVASTGYVVEPYRKYPTLLEVLLNFLKTEQNQGTRREAIRVLGLLGALDPYKHKVNIGMIDQSRDASAVSLSESKSSQDSSDYSTSEMLVNMGNLPLDEFYPAVSMVALMRIFRDQSLSHHHTMVVQAITFIFKSLGLKCVQFLPQVMPTFLNVIRVCDGAIREFLFQQLGMLVSFVKSHIRPYMDEIVTLMREFWVMNTSIQSTIILLIEQIVVALGGEFKLYLPQLIPHMLRVFMHDNSQGRIVSIKLLAAIQLFGANLDDYLHLLLPPIVKLFDAPEVPLPSRKAALETVDRLTESLDFTDYASRIIHPIVRTLDQSPELRSTAMDTLSSLVFQLGKKYQIFIPMVNKVLVRHRINHQRYDVLICRIVKGYTLADEEEDPLIYQHRMLRSSQGDALASGPVETGPMKKLHVSTINLQKAWGAARRVSKDDWLEWLRRLSLELLKDSSSPSLRSCWALAQAYNPMARDLFNAAFVSCWSELNEDQQDELIRSIELALTSQDIAEVTQTLLNLAEFMEHSDKGPLPLRDDNGIVLLGERAAKCRAYAKALHYKELEFQKGPTPAILESLISINNKLQQPEAASGVLEYAMKHFGELEIQATWYEKLHEWEDALVAYDKKMDTNKEDPELMLGRMRCLEALGEWGQLHQQCCEKWTLVNDETQAKMARMAAAAAWGLGQWDSMEEYTCMIPRDTHDGAFYRAVLALHQDLFSLAQQCIDKARDLLDAELTAMAGESYSRAYGAMVSCHMLSELEEVIQYKLVPERREIIRQIWWERLQGCQRIVEDWQKILMVRSLVVSPHEDMRTWLKYASLCGKSGRLALAHKTLVLLLGVDPSRQLDHPLPTAHPQVTYAYMKNMWKSARKIDAFQHMQHFVQTMQQQAQHAIATEDQQHKQELHKLMARCFLKLGEWQLNLQGINESTIPKVLQYYSAATEHDRSWYKAWHAWAVMNFEAVLHYKHQNQARDEKKKLRHASGANITNATTAATTAASAAAATSTEGSNSESEAESNENSPTPSPLQKKVTEDLSKTLLLYTVPAVQGFFRSISLSRGNNLQDTLRVLTLWFDYGHWPDVNEALVEGVKAIQIDTWLQVIPQLIARIDTPRPLVGRLIHQLLTDIGRYHPQALIYPLTVASKSTTTARHNAANKILKNMCEHSNTLVQQAMMVSEELIRVAILWHEMWHEGLEEASRLYFGERNVKGMFEVLEPLHAMMERGPQTLKETSFNQAYGRDLMEAQEWCRKYMKSGNVKDLTQAWDLYYHVFRRISKQLPQLTSLELQYVSPKLLMCRDLELAVPGTYDPNQPIIRIQSIAPSLQVITSKQRPRKLTLMGSNGHEFVFLLKGHEDLRQDERVMQLFGLVNTLLANDPTSLRKNLSIQRYAVIPLSTNSGLIGWVPHCDTLHALIRDYREKKKILLNIEHRIMLRMAPDYDHLTLMQKVEVFEHAVNNTAGDDLAKLLWLKSPSSEVWFDRRTNYTRSLAVMSMVGYILGLGDRHPSNLMLDRLSGKILHIDFGDCFEVAMTREKFPEKIPFRLTRMLTNAMEVTGLDGNYRTTCHTVMEVLREHKDSVMAVLEAFVYDPLLNWRLMDTNTKGNKRSRTRTDSYSAGQSVEILDGVELGEPAHKKAGTTVPESIHSFIGDGLVKPEALNKKAIQIINRVRDKLTGRDFSHDDTLDVPTQVELLIKQATSHENLCQCYIGWCPFW

SEQ ID NO: 9 - protein, 1161 aa, Mus musculus
MAQLANIGELLSMLDSSTLGVRDDVTAIFKESLNSERGPMLVNTLVDYYLETNSQPVLHILTTLQEPHDKHLLDKINEYVGKAATRLSILSLLGHVVRLQPSWKHKLSQAPLLPSLLKCLKMDTDVVVLTTGVLVLITMLPMIPQSGKQHLLDFFDIFGRLSSWCLKKPGHVTEVYLVHLHASVYALFHRLYGMYPCNFVSFLRSHYSMKENVETFEEVVKPMMEHVRIHPELVTGSKDHELDPRRWKTLETHDVVIECAKISLDPTEASYEDGYSVSHQLSACFPYRSADVTTSPYVDTQNSYGGSTSTPSSSSRLMLFSPPGQLPQSLSSPSTRLLPEPLQASLWSPSAVCGMTTPPTSPGNVPADLSHPYSKAFGTTAGGKGTPSGTPATSPPPAPPCPQDDCVHGSAAQASATAPRKEERADSSRPYLHRQSNDRGLEDPPGSKGSVTLRNLPDFLGDLASEEDSIEKDKEEAAISKELSEITTAEADPVVPRGGFDSPFYRDSLSGSQRKTHSAASGTQGSSVNPEPLHSSLDKHGPDTPKQAFTPIDPPSGSADVSPAGDRDRQTSLETSILTPSPCKIPPQRGVSFGSGQLPPYDHLFEVALPKTACHFVSKKTEELLKKVKGNPEEDCVPSTSPMEVLDRLIEQGAGAHSKELSRLSLPSKSVDWTHFGGSPPSDELRTLRDQLLLLHNQLLYERFKRQQHALRNRRLLRKVIRAAALEEHNAAMKDQLKLQEKDIQMWKVSLQKEQARYSQLQEQRDTMVTQLHSQIRQLQHDREEFYNQSQELQTKLEDCRNMIAELRVELKKANNKVCHTELLLSQVSQKLSNSESVQQQMEFLNRQLLVLGEVNELYLEQLQSKHPDTTKEVEMMKTAYRKELEKNRSHLLQQNQRLDASQRRVLELESLLAKKDHLLLEQKKYLEDVKSQASGQLLAAESRYEAQRKITRVLELEILDLYGRLEKDGRLRKLEEDRAEAAEAAEERLDCCSDGCTDSLVGHNEEASGHNGETRTSRPGGTRASCGGRVTGGSSSSSSELSTPEKPPSQRFSSRWEPALGEPSSSIPTTVGSLPSSKSFLGMKARELFRNKSESQCDEDSVTMSSSSLSETLKTELGKDSGTENKTSLSLDAPHPSSPNSDNVGQLHIMDYNETHPEHS

SEQ ID NO: 10 - protein, 1740 aa, Homo sapiens
MAKPTSKDSGLKEKFKILLGLGTPRPNPRSAEGKQTEFIITAEILRELSMECGLNNRIRMIGQICEVAKTKKFEEHAVEALWKAVADLLQPERPLEARHAVLALLKAIVQGQGERLGVLRALFFKVIKDYPSNEDLHERLEVFKALTDNGRHITYLEEELADFVLQWMDVGLSSEFLLVLVNLVKFNSCYLDEYIARMVQMICLLCVRTASSVDIEVSLQVLDAVVCYNCLPAESLPLFIVTLCRTINVKELCEPCWKLMRNLLGTHLGHSAIYNMCHLMEDRAYMEDAPLLRGAVFFVGMALWGAHRLYSLRNSPTSVLPSFYQAMACPNEVVSYEIVLSITRLIKKYRKELQVVAWDILLNIIERLLQQLQTLDSPELRTIVHDLLTTVEELCDQNEFHGSQERYFELVERCADQRPESSLLNLISYRAQSIHPAKDGWIQNLQALMERFFRSESRGAVRIKVLDVLSFVLLINRQFYEEELINSVVISQLSHIPEDKDHQVRKLATQLLVDLAEGCHTHHFNSLLDIIEKVMARSLSPPPELEERDVAAYSASLEDVKTAVLGLLVILQTKLYTLPASHATRVYEMLVSHIQLHYKHSYTLPIASSIRLQAFDFLLLLRADSLHRLGLPNKDGVVRFSPYCVCDYMEPERGSEKKTSGPLSPPTGPPGPAPAGPAVRLGSVPYSLLFRVLLQCLKQESDWKVLKLVLGRLPESLRYKVLIFTSPCSVDQLCSALCSMLSGPKTLERLRGAPEGFSRTDLHLAVVPVLTALISYHNYLDKTKQREMVYCLEQGLIHRCASQCVVALSICSVEMPDIIIKALPVLVVKLTHISATASMAVPLLEFLSTLARLPHLYRNFAAEQYASVFAISLPYTNPSKFNQYIVCLAHHVIAMWFIRCRLPFRKDFVPFITKGLRSNVLLSFDDTPEKDSFRARSTSLNERPKRIQTSLTSASLGSADENSVAQADDSLKNLHLELTETCLDMMARYVFSNFTAVPKRSPVGEFLLAGGRTKTWLVGNKLVTVTTSVGTGTRSLLGLDSGELQSGPESSSSPGVHVRQTKEAPAKLESQAGQQVSRGARDRVRSMSGGHGLRVGALDVPASQFLGSATSPGPRTAPAAKPEKASAGTRVPVQEKTNLAAYVPLLTQGWAEILVRRPTGNTSWLMSLENPLSPFSSDINNMPLQELSNALMAAERFKEHRDTALYKSLSVPAASTAKPPPLPRSNTDSAVVMEEGSPGEVPVLVEPPGLEDVEAALGMDRRTDAYSRSSSVSSQEEKSLHAEELVGRGIPIERVVSSEGGRPSVDLSFQPSQPLSKSSSSPELQTLQDILGDPGDKADVGRLSPEVKARSQSGTLDGESAAWSASGEDSRGQPEGPLPSSSPRSPSGLRPRGYTISDSAPSRRGKRVERDALKSRATASNAEKVPGINPSFVFLQLYHSPFFGDESNKPILLPNESQSFERSVQLLDQIPSYDTHKIAVLYVGEGQSNSELAILSNEHGSYRYTEFLTGLGRLIELKDCQPDKVYLGGLDVCGEDGQFTYCWHDDIMQAVFHIATLMPTKDVDKHRCDKKRHLGNDFVSIVYNDSGEDFKLGTIKGQFNFVHVIVTPLDYECNLVSLQCRKDMEGLVDTSVAKIVSDRNLPFVARQMALHANMASQVHHSRSNPTDIYPSKWIARLRHIKRLRQRICEEAAYSNPSLPLVHPPSHSKAPAQTPAEPTPGYEVGQRKRLISSVEDFTEFV

SEQ ID NO: 11 - protein, 501 aa, Mus musculus
MAAAAATAAAAAAPSGGGGGGEEERLEEKSEDQDLQGLRDKPLKFKKAKKDKKEDKEGKHEPLQPSAHHSAEPAEAGKAETSESSGSAPAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPPKKPKSPKAPGTGRGRGRPKGSGTGRPKAAASEGVQVKRVLEKSPGKLVVKMPFQASPGGKGEGGGATTSAQVMVIKRPGRKRKAEADPQAIPKKRGRKPGSVVAAAAAEAKKKAVKESSIRSVHETVLPIKKRKTRETVSIEVKEVVKPLLVSTLGEKSGKGLKTCKSPGRKSKESSPKGRSSSASSPPKKEHHHHHHHSESTKAPMPLLPSPPPPEPESSEDPISPPEPQDLSSSICKEEKMPRGGSLESDGCPKEPAKTQPMVATTTTVAEKYKHRGEGERKDIVSSSMPRPNREEPVDSRTPVTERVS

SEQ ID NO: 12 - protein, 403 aa, Mus musculus
MTAIIKEIVSRNKRRYQEDGFDLDLTYIYPNIIAMGFPAERLEGVYRNNIDDVVRFLDSKHKNHYKIYNLCAERHYDTAKFNCRVAQYPFEDHNPPQLELIKPFCEDLDQWLSEDDNHVAAIHCKAGKGRTGVMICAYLLHRGKFLKAQEALDFYGEVRTRDKKGVTIPSQRRYVYYYSYLLKNHLDYRPVALLFHKMMFETIPMFSGGTCNPQFVVCQLKVKIYSSNSGPTRREDKFMYFEFPQPLPVCGDIKVEFFHKQNKMLKKDKMFHFWVNTFFIPGPEETSEKVENGSLCDQEIDSICSIERADNDKEYLVLTLTKNDLDKANKDKANRYFSPNFKVKLYFTKTVEEPSNPEASSSTSVTPDVSDNEPDHYRYSDTTDSDPENEPFDEDQHSQITKV

SEQ ID NO: 13 - protein, 591 aa, Mus musculus
MRAGRGGVPGSGGLRAPPPPLLLLLLAMLPAAAPRSPALAAAPAGPSVSLYLSEDEVRRLLGLDAELYYVRNDLISHYALSFNLLVPSETNFLHFTWHAKSKVEYKLGFQVDNFVAMGMPQVNISAQGEVPRTLSVFRVELSCTGKVDSEVMILMQLNLTVNSSKNFTVLNFKRRKMCYKKLEEVKTSALDKNTSRTIYDPVHAAPTTSTRVFYISVGVCCAVIFLVAIILAVLHLHSMKRIELDDSISASSSSQGLSQPSTQTTQYLRADTPNNATPITSYPTLRIEKNDLRSVTLLEAKAKVKDIAISRERITLKDVLQEGTFGRIFHGILVDEKDPNKEKQTFVKTVKDQASEVQVTMMLTESCKLRGLHHRNLLPITHVCIEEGEKPMVVLPYMNWGNLKLFLRQCKLVEANNPQAISQQDLVHMAIQIACGMSYLARREVIHRDLAARNCVIDDTLQVKITDNALSRDLFPMDYHCLGDNENRPVRWMALESLVNNEFSSASDVWAFGVTLWELMTLGQTPYVDIDPFEMAAYLKDGYRIAQPINCPDELFAVMACCWALDPEERPKFQQLVQCLTEFHAALGAYV

SEQ ID NO: 14 - protein, 2582 aa, Mus musculus
MADPIMDLFDDPNLFGLDSLTDDSFNQVTQDPIEEALGLPSSLDSLDQMNQDGGGGDVGNSSASDLVPPPEETASTELPKESTAPAPESLTLHDYTTQPTSQEQPAQPVLQTSTPTAGLLQVSKSQEILSQGNPFMGVSATGVSPSNTGGQPSQSAPKIVILKAPPNSSVTGTHVAQIQAQGITSTAQPLVAGTANGGKVTFTKVLTGTPLRPGVSIVSGNTVLATKVPGNQAAVQRIVQPSRPVKQLVLQPVKGSAPAGNPGAAGPPLKPAVTLTSTPTQGESKRITLVLQQPQSGGPQGHRHVVLGSLPGKIVLQGNQLAALTQAKNAQGQPAKVVTIQLQVQQPQQKIQIVPQPPSSQPQPQPQPPPSAQPLTLSSVQQAQIMGPGQNPGQRLSVPLKMVLQPQAGSSQGASSGLSVVKVLSASEVAALSSPASCAPHTAGKTGMEENRRLEHQKKQEKANRIVAEAIARARARGEQNIPRVLNEDELPSVRPEEEGEKKRRKKSSGERLKEEKPKKSKTAAASKTKGKSKLNTITPVVGKKRKRNTSSDNSDVEVMPAQSPREDEESSIQKRRSNRQVKRKKYTEDLDIKITDDEEEEEVDVTGPIKPEPILPEPVQEPDGETLPSMQFFVENPSEEDAAIVDKVLSMRVVKKELPSGQYTEAEEFFVKYKNYSYLHCEWATISQLEKDKRIHQKLKRFKTKMAQMRHFFHEDEEPFNPDYVEVDRILDESHSVDKDNGEPVIYYLVKWCSLPYEDSTWELKEDVDEGKIREFKRIQSRHPELRRVNRPQANAWKKLELSHEYKNRNQLREYQLEGVNWLLFNWYNRQNCILADEMGLGKTIQSIAFLQEVYNVGIHGPFLVIAPLSTITNWEREFNTWTEMNTIVYHGSLASRQMIQQYEMYCKDSRGRLIPGAYKFDALITTFEMILSDCPELREIEWRCVIIDEAHRLKNRNCKLLDSLKHMDLEHKVLLTGTPLQNTVEELFSLLHFLEPSQFPSESEFLKDFGDLKTEEQVQKLQAILKPMMLRRLKEDVEKNLAPKQETIIEVELTNIQKKYYRAILEKNFSFLSKGAGHTNMPNLLNTMMELRKCCNHPYLINGAEEKILMEFREACHIIPQDFHLQAMVRSAGKLVLIDKLLPKLKAGGHKVLIFSQMVRCLDILEDYLIQRRYLYERIDGRVRGNLRQAAIDRFSKPDSDRFVFLLCTRAGGLGINLTAADTCIIFDSDWNPQNDLQAQARCHRIGQSKAVKVYRLITRNSYEREMFDKASLKLGLDKAVLQSMSGRDGNITGIQQFSKKEIEDLLRKGAYAAIMEEDDEGSKFCEEDIDQILLRRTTTITIESEGKGSTFAKASFVASENRTDISLDDPNFWQKWAKKADLDMDLLNSKNNLVIDTPRVRKQTRHFSTLKDDDLVEFSDLESEDDERPRSRRHDRHHTYGRTDCFRVEKHLLVYGWGRWRDILSHGRFKRRMTERDVETICRAILVYCLLHYRGDENIKSFIWDLISPAENGKTKELQNHSGLSIPVPRGRKGKKVKSQSTFDIHKADWIRKYNPDTLFQDESYKKHLKHQCNKVLLRVRMLYYLRQEVIGDQAEKVLGGAIASEIDIWFPVVDQLEVPTTWWDSEADKSLLIGVFKHGYEKYNTMRADPALCFLEKAGRPDDKAIAAEHRVLDNFSDLVEGIDFDKDCEDPEYKPLQGPPKDPDDEGDPLMMMDEEISVIDGEEAQVTQQPGHLFWPPGSALTARLRRLVTAYQRSYKREQMKMEAAERGDRRRRRCEAAFKLKEIARREKQQRWTRREQTDFYRVVSTFGVEYDPDNMQFHWDRFRTFARLDKKTDESLTKYFHGFVAMCRQVCRLPPAAGDEPPDPNLFIEPITEERASRTLYRIELLRRLREQVLCHPLLEDRLALCQPPGLELPKWWEPVRHDGELLRGAARHGVSQTDCNIMQDPDFSFLAARMNYMQNHQAGASAASLSRCSTPLLHQQCTSRTASPSPLRPDAPVEKSPEESTVQVPNLESLTLKLEDEVVARSRLTSQDYEVRVGSSDTAPLSRSVPPVKLEDEDDSDSELDLSKLSPSSSSSSSSSSSSSSTDESEDEKEEKLTADRSRPKLYDEESLLSLTMSQDGFPNEDGEQMTPELLLLQERQRASEWPKDRVLINRIDLVCQAVLSGKWPSNRRSQEVTAGGILGPGNHLLDSPSLTPGEDGDSPVPTPRSGSAASMAEEEASAVTTAAAQFTKLRRGMDEKEFTVQIKDEEGLKLTFQKHRLMANGVMGDGHPLFHKKKGNRKKLVELEVECMEEPNHLDLDLETRIPVINKVDGTLLVGDEAPRRAELEMWLQGHPEFAVDPRFLAYMEERRKQKWQRCKKNNKAELNCLGMEPVQPANSRNGKKGHYAETAFNRVLPGPVAPENSKKRVRRTRPDLSKMMALMQGGSTGSLSLHNTFQHSSSNLQSVSSLGHSSTTSASLPFMPFVMGAAAPPHVDSSTMLHHHHHHPHPHHHHHHHPGLRTTGYPSSPATTTSGTALRLPTLQPEDDDEEEDEEDDDLSQGYDSSERDFSLIDDPMMPANSDSSEDADD

SEQ ID NO: 15 - protein, 1292 aa, Mus musculus
MKLATGLWVWGSLLMAAGTVQPSASQSVCAGTENKLSSLSDLEQQYRALRKYYENCEVVMGNLEITSIEHNRDLSFLRSIREVTGYVLVALNQFRYLPLENLRIIRGTKLYEDRYALAIFLNYRKDGNFGLQELGLKNLTEILNGGVYVDQNKFLCYADTIHWQDIVRNPWPSNMTLVSTNGSSGCGRCHKSCTGRCWGPTENHCQTLTRTVCAEQCDGRCYGPYVSDCCHRECAGGCSGPKDTDCFACMNFNDSGACVTQCPQTFVYNPTTFQLEHNFNAKYTYGAFCVKKCPHNFVVDSSSCVRACPSSKMEVEENGIKMCKPCTDICPKACDGIGTGSLMSAQTVDSSNIDKFINCTKINGNLIFLVTGIHGDPYNAIDAIDPEKLNVFRTVREITGFLNIQSWPPNMTDFSVFSNLVTIGGRVLYSGLSLLILKQQGITSLQFQSLKEISAGNIYITDNSNLCYYHTINWTTLFSTINQRIVIRDNRRAENCTAEGMVCNHLCSNDGCWGPGPDQCLSCRRFSRGKICIESCNLYDGEFREFENGSICVECDSQCEKMEDGLLTCHGPGPDNCTKCSHFKDGPNCVEKCPDGLQGANSFIFKYADQDRECHPCHPNCTQGCNGPTSHDCIYYPWTGHSTLPQHARTPLIAAGVIGGLFILVIMALTFAVYVRRKSIKKKRALRRFLETELVEPLTPSGTAPNQAQLRILKETELKRVKVLGSGAFGTVYKGIWVPEGETVKIPVAIKILNETTGPKANVEFMDEALIMASMDHPHLVRLLGVCLSPTIQLVTQLMPHGCLLDYVHEHKDNIGSQLLLNWCVQIAKGMMYLEERRLVHRDLAARNVLVKSPNHVKITDFGLARLLEGDEKEYNADGGKMPIKWMALECIHYRKFTHQSDVWSYGVTIWELMTFGGKPYDGIPTREIPDLLEKGERLPQPPICTIDVYMVMVKCWMIDADSRPKFKELAAEFSRMARDPQRYLVIQGDDRMKLPSPNDSKFFQNLLDEEDLEDMMDAEEYLVPQAFNIPPPIYTSRTRIDSNRNQFVYQDGGFATQQGMPMPYRATTSTIPEAPVAQGATAEMFDDSCCNGTLRKPVAPHVQEDSSTQRYSADPTVFAPERNPRGELDEEGYMTPMHDKPKQEYLNPVEENPFVSRRKNGDLQALDNPEYHSASSGPPKAEDEYVNEPLYLNTFANALGSAEYMKNSVLSVPEKAKKAFDNPDYWNHSLPPRSTLQHPDYLQEYSTKYFYKQNGRIRPIVAENPEYLSEFSLKPGTMLPPPPYRHRNTVV

SEQ ID NO: 16 - protein, 2009 aa, Mus musculus
MEQTVLVPPGPDSFNFFTRESLAAIERRIAEEKAKNPKPDKKDDDENGPKPNSDLEAGKNLPFIYGDIPPEMVSEPLEDLDPYYINKKTFIVLNKGKAIFRFSATSALYILTPFNPLRKIAIKILVHSLFSMLIMCTILTNCVFMTMSNPPDWTKNVEYTFTGIYTFESLIKIIARGFCLEDFTFLRDPWNWLDFTVITFAYVTEFVDLGNVSALRTFRVLRALKTISVIPGLKTIVGALIQSVKKLSDVMILTVFCLSVFALIGLQLFMGNLRNKCVQWPPTNASLEEHSIEKNITMDYNGTLVNETVFEFDWKSYIQDSRYHYFLEGVLDALLCGNSSDAGQCPEGYMCVKAGRNPNYGYTSFDTFSWAFLSLFRLMTQDFWENLYQLTLRAAGKTYMIFFVLVIFLGSFYLINLILAVVAMAYEEQNQATLEEAEQKEAEFQQMLEQLKKQQEAAQQAAATTASEHSREPSAAGRLSDSSSEASKLSSKSAKERRNRRKKRKQKEQSGGEEKDDDEFHKSESEDSIRRKGFRFSIEGNRLTYEKRYSSPHQSLLSIRGSLFSPRRNSRTSLFSFRGRAKDVGSENDFADDEHSTFEDNESRRDSLFVPRRHGERRNSNLSQTSRSSRMLAVFPANGKMHSTVDCNGVVSLVGGPSVPTSPVGQLLPEVIIDKPATDDNGTTTETEMRKRRSSSFHVSMDFLEDPSQRQRAMSIASILTNTVEELEESRQKCPPCWYKFSNIFLIWDCSPYWLKVKHIVNLVVMDPFVDLAITICIVLNTLFMAMEHYPMTEHFNHVLTVGNLVFTGIFTAEMFLKIIAMDPYYYFQEGWNIFDGFIVTLSLVELGLANVEGLSVLRSFRLLRVFKLAKSWPTLNMLIKIIGNSVGALGNLTLVLAIIVFIFAVVGMQLFGKSYKDCVCKIATDCKLPRWHMNDFFHSFLIVFRVLCGEWIETMWDCMEVAGQAMCLTVFMMVMVIGNLVVLNLFLALLLSSFSADNLAATDDDNEMNNLQIAVDRMHKGIAYVKRKIYEFIQQSFVKKQKILDEIKPLDDLNNRKDNCISNHTTEIGKDLDCLKDVNGTTSGIGTGSSVEKYIIDESDYMSFINNPSLTVTVPIAVGESDFENLNTEDFSSESDLEESKEKLNESSSSSEGSTVDIGAPAEEQPVIEPEETLEPEACFTEGCVQRFKCCQISVEEGRGKQWWNLRRTCFRIVEHNWFETFIVFMILLSSGALAFEDIYIDQRKTIKTMLEYADKVFTYIFILEMLLKWVAYGYQTYFTNAWCWLDFLIVDVSLVSLTANALGYSELGAIKSLRTLRALRPLRALSRFEGMRVVVNALLGAIPSIMNVLLVCLIFWLIFSIMGVNLFAGKFYHCVNTTTGDIFEISEVNNHSDCLKLIERNETARWKNVKVNFDNVGFGYLSLLQVATFKGWMDIMYAAVDSRNVELQPKYEESLYMYLYFVIFIIFGSFFTLNLFIGVIIDNFNQQKKKFGGQDIFMTEEQKKYYNAMKKLGSKKPQKPIPRPGNKFQGMVFDFVTRQVFDISIMILICLNMVTMMVETDDQSDYVTSILSRINLVFIVLFTGECVLKLISLRHYYFTIGWNIFDFVVVILSIVGMFLAELIEKYFVSPTLFRVIRLARIGRILRLIKGAKGIRTLLFALMMSLPALFNIGLLLFLVMFIYAIFGMSNFAYVKREVGIDDMFNFETFGNSMICLFQITTSAGWDGLLAPILNSKPPDCDPNKVNPGSSVKGDCGNPSVGIFFFVSYIIISFLVVVNMYIAVILENFSVATEESAEPLSEDDFEMFYEVWEKFDPDATQFMEFEKLSQFAAALEPPLNLPQPNKLQLIAMDLPMVSGDRIHCLDILFAFTKRVLGESGEMDALRIQMEERFMASNPSKVSYQPITTTLKRKQEEVSAVIIQRAYRRHLLKRTVKQASFTYNKNKLKGGANLLVKEDMLIDRINENSITEKTDLTMSTAACPPSYDRVTKPIVEKHEQEGKDEKAKGK

SEQ ID NO: 17 - protein, 655 aa, Mus musculus
MRPSGTARTTLLVLLTALCAAGGALEEKKVCQGTSNRLTQLGTFEDHFLSLQRMYNNCEVVLGNLEITYVQRNYDLSFLKTIQEVAGYVLIALNTVERIPLENLQIIRGNALYENTYALAILSNYGTNRTGLRELPMRNLQEILIGAVRFSNNPILCNMDTIQWRDIVQNVFMSNMSMDLQSHPSSCPKCDPSCPNGSCWGGGEENCQKLTKIICAQQCSHRCRGRSPSDCCHNQCAAGCTGPRESDCLVCQKFQDEATCKDTCPPLMLYNPTTYQMDVNPEGKYSFGATCVKKCPRNYVVTDHGSCVRACGPDYYEVEEDGIRKCKKCDGPCRKVCNGIGIGEFKDTLSINATNIKHFKYCTAISGDLHILPVAFKGDSFTRTPPLDPRELEILKTVKEITGFLLIQAWPDNWTDLHAFENLEIIRGRTKQHGQFSLAVVGLNITSLGLRSLKEISDGDVIISGNRNLCYANTINWKKLFGTPNQKTKIMNNRAEKDCKAVNHVCNPLCSSEGCWGPEPRDCVSCQNVSRGRECVEKCNILEGEPREFVENSECIQCHPECLPQAMNITCTGRGPDNCIQCAHYIDGPHCVKTCPAGIMGENNTLVWKYADANNVCHLCHANCTYGCAGPGLQGCEVWPSGYVQWQWILKTFWI

SEQ ID NO: 18 - protein, 422 aa, Mus musculus
MRNIFKRNQEPNVAPATTTATMPLAPVAPADNSTESTGPGESQEDMFAKLKEKFFNEINKIPLPPWALIAMAVVAGLLLLTCCFCICKKCCCKKKKNKKEKGKGMKNAMNMKDMKGGQDDDDAETGLTEGEGEGEEEKEPENLGKLQFSLDYDFQANQLTVGVLQAAELPALDMGGTSDPYVKVFLLPDKKKKYETKVHRKTLNPAFNETFTFKVPYQELGGKTLVMAIYDFDRFSKHDIIGEVKVPMNTVDLGQPIEEWRDLQGGEKEEPEKLGDICTSLRYVPTAGKLTVCILEAKNLKKMDVGGLSDPYVKIHLMQNGKRLKKKKTTVKKKTLNPYFNESFSFEIPFEQIQKVQVVVTVLDYDKLGKNEAIGKIFVGSNATGTELRHWSDMLANPRRPIAQWHSLKPEEEVDALLGKNK

SEQ ID NO: 19 - protein, 1544 aa, Mus musculus
METSAPAPALEKKEAKSGLLEDSSFPDPGKKACPLAVAAAVAAHGVPQQLLPAFHAPLPIDMRHQEGRYHYDPHSVHSVHGPPTLSGSPVISDISLIRLSPHPAGPGESPFSAHHPYVNPHMEHYLRSVHSSPTLSMISAARGLSPADVAHEHLKERGLFSLAAPGTNPSDYYHQMTLMASHPTPYGDLLMQSGGAASAPHLHDYLNPVDASRFSSPRVTPRLSRKRALSISPLSDASLDLQRMIRTSPNSLVAYINNSRSSSAASGSYGHLSAGALSPAFTFPHPINPVAYQQILSQQRGLGSAFGHTPPLIQPSPTFLAQQPMTLTSISTMPTQLSSSSSNCLNDANQNKQNSESAVSSTVNPITIHKRSKVKTEAEGLRPASPLGLTQEQLADLKEDLDRDDCKQEAEVVIYETNCHWADCTKEYDTQEQLVHHINNEHIHGEKKEFVCRWQACTREQKPFKAQYMLVVHMRRHTGEKPHKCTFEGCSKAYSRLENLKTHLRSHTGEKPYVCEHEGCNKAFSNASDRAKHQNRTHSNEKPYICKIPGCTKRYTDPSSLRKHVKTVHGPDAHVTKKQRNDVHVRAPLLKENGDNEASAEPGGRGPEESVEASSTSHTVEDCLHIKAIKTESSGLCQSSPGAQSSCSSEPSPLGSAPNNDSGMEMPGTGPGSLGDLTALADTCPGADTSALAAPSTGGLQLRKHMSTVHRFEQLKREKLKSLKDSCSWAGPAPHTRNTKLPPLPVNGSVLENFNNTGGGGPAGLLPSQRLPELTEVTMLSQLQERRDSSTSTMSSAYTVSRRSSGISPYFSSRRSSEASPLGGLRPHNASSADSYDPISTDASRRSSEASQCSGGGPGLLNLTPAQQYNLRAKYAAATGGPPPTPLPGLDRVSLRTRLALLDAPERALPGACPHPLGPRRGSDGPTYSHGHGHGYAGAAPAFPHEGPNSSTRRASDPVRRPDPLILPRVQRFHSTHNMNPGSLPPCADRRGLHVQSHPSVDSNLTRNAYSPRPPSINENVVMEAVAAGVDGPGLECDLGLVEDELVLPDDVVQYIKAHTGGTLDDGIRQGYPTEGTGFPENSKLPSPGLQGHRRLAAADSNMGPSAPGLGGCQLSYSPSSNLNKSNMPVQWNEVSSGTVDALPTQVKPPPFPHSNLAVVQQKPAFGQYPGYNPQSVQSSSGGLDSTQPHLQLRGAPSASRGSYTQQPRQPAAGSQCLGMSAAMSPQASYSQAHPQLSPNIVSGSLNQFSPSCSNMAAKPSHLGLPQQMEVVPNATIMNGHQREHGVPNSSLAAVSQPHPVLSYPQQDSYQQGSNLLSSHQPGFMESQQNAGFGLMQPRPPLEPNTASRHRGVRSGQQQLYARTTGQAMVTSANQETAEAMPKGPAGTMVSLAPQPSQDTGRAQDQNTLYYYGQIHMYEQNGGCPAVQPQPPQPQACSDSIQPEPLPSPGVNQVSSTVDSQLLEPPQIDFDAIMDDGDHSSLFSGALSPTLLHNLSQNSSRLTTPRNSLTLPSIPAGISNMAVGDMSSMLTSLAEESKFLNMMT

SEQ ID NO: 20 - protein, 365 aa, Mus musculus
MPSISSDRAALCAGCGGKISDRYYLLAVDKQWHMRCLKCCECKLNLESELTCFSKDGSIYCKEDYYRRFSVQRCARCHLGISASEMVMRARDLVYHLNCFTCTTCNKMLTTGDHFGMKDSLVYCRLHFEALLQGEYPAHFNHADVAAAAAAAAAAKSAGLGSAGANPLGLPYYNGVGTVQKGRPRKRKSPGPGADLAAYNAALSCNENDAEHLDRDQPYPSSQKTKRMRTSFKHHQLRTMKSYFAINHNPDAKDLKQLAQKTGLTKRVLQVWFQNARAKFRRNLLRQENTGVDKTSDATLQTGTPSGPASELSNASLSPSSTPTTLTDLTSPTLPTVTSVLTSVPGNLEGHEPHSPSQTTLTNLF

(19) United States
(12) Patent Application Publication
(10) Pub. No.: US 2024/0400985 A1
(43) Pub. Date: Dec. 5, 2024

(54) METHODS FOR GENERATING PARVALBUMIN-POSITIVE INTERNEURONS

(71) Applicants: The Regents of the University of California, Oakland, CA (US); CZ Biohub SF, LLC, San Francisco, CA (US)

(72) Inventors: Mohammed Andres Mostajo Radji, Santa Cruz, CA (US); Alexander A. Pollen, San Francisco, CA (US)

(21) Appl. No.: 18/563,301

(22) PCT Filed: Jun. 14, 2022
(86) PCT No.: PCT/US22/33434

§ 371 Date: Nov. 21, 2023

Related U.S. Application Data
(60) Provisional application No. 63/210,742, filed on Jun. 15, 2021.

(51) Int. Cl. C12N 5/0793

(52) U.S. Cl. CPC C12N 5/0619; C12N 2506/08

(57) ABSTRACT

The present disclosure provides a method of generating an enriched population of parvalbumin-positive interneurons. The present disclosure provides a chimeric organoid comprising an enriched population of parvalbumin-positive interneurons. The present disclosure provides methods of identifying agents that modulate a feature of a parvalbumin-positive interneuron.

### CROSS-REFERENCE

This application claims the benefit of U.S. Provisional Patent Application No. 63/210,742, filed Jun. 15, 2021, which application is incorporated herein by reference in its entirety.

### STATEMENT REGARDING FEDERALLY SPONSORED RESEARCH

This invention was made with government support under Grant No. TL1 TR001871 awarded by the National Institutes for Health. The government has certain rights in the invention.

### INCORPORATION BY REFERENCE OF SEQUENCE LISTING PROVIDED AS A TEXT FILE

A Sequence Listing is provided herewith as a text file, “CZBH-002WO_SEQ_LIST_ST25.txt” created on Jun. 13, 2022, and having a size of 153 KB. The contents of the text file are incorporated by reference herein in their entirety.

### INTRODUCTION

Parvalbumin-positive interneurons are the largest subpopulation of inhibitory interneurons in the cerebral cortex, and are involved in a variety of neurodevelopmental and neurodegenerative disorders.

There is a need in the art for method of generating parvalbumin-positive interneurons.

### SUMMARY

The present disclosure provides a method of generating an enriched population of parvalbumin-positive interneurons. The present disclosure provides a chimeric brain organoid comprising an enriched population of parvalbumin-positive interneurons. The present disclosure provides methods of identifying agents that modulate a feature of a parvalbumin-positive interneuron.

### BRIEF DESCRIPTION OF THE DRAWINGS

FIG. 1A-1T provide amino acid sequences of polypeptide gene products of various target nucleic acids of interest.

FIG. 2: Host-dependent differentiation of mouse MGE progenitors. Transplantation of mouse MGE progenitors into mouse and human cortical organotypic cultures give rise to different interneuron populations 7 DPT. A) Transplantation into E14.5 mouse organotypic hosts give rise primarily to SST-positive interneurons, while transplantation into GW22 human organotypic hosts gives rise to PV-positive interneurons. B) Quantifications of interneuron populations in mouse and human hosts. C) Transplantation of unlabeled MGE progenitors allows for unbiased identification of PV-positive interneurons (Ins). White arrows indicate mouse cells, while yellow arrows indicate neighboring human cells.

FIG. 3A-3D provide data on chimeric corticoid organoid generation and migratory behavior of mouse inhibitory neurons.

FIG. 4A-4C provide data on mouse inhibitory neuron integration into human chimeric cortical organoid models.

FIG. 5A-5H provide data showing that organoid models recapitulate species-specific fate biases on transplanted INs.

FIG. 6A-6F provide data depicting the effect of cellular environment on mouse inhibitory neuron fate.

FIG. 7A-7D provide data on the necessity of a 3-dimensional human cortical environment for the instruction of PV fate.

FIG. 8A-8B provide data showing that PV fate is instructed in a non cell autonomous mechanism.

FIG. 9A-9C provide data on the reprogramming of postmitotic SST INs into a PV fate.

FIG. 10 provide disease-associated mutations of interest.

### DEFINITIONS

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a parvalbumin-positive interneuron” includes a plurality of such parvalbumin-positive interneurons and reference to “the target nucleic acid” includes reference to one or more target nucleic acids and equivalents thereof known to those skilled in the art, and so forth. It is further noted that the claims may be drafted to exclude any optional element. As such, this statement is intended to serve as antecedent basis for use of such exclusive terminology as “solely.” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides a method of generating an enriched population of parvalbumin-positive interneurons. The present disclosure provides a chimeric cortical organoid comprising an enriched population of parvalbumin-positive interneurons. The present disclosure provides methods of identifying agents that modulate a feature of a parvalbumin-positive interneuron.

### Methods For Generating Parvalbumin-Positive Interneurons

The present disclosure provides a method of generating an enriched population of parvalbumin-positive interneurons. The methods generally involve culturing a population of mouse primary neuronal progenitors in a human brain organoid or a primary brain slice of human origin. Over a period of time, the mouse primary neuronal progenitors differentiate into parvalbumin-positive interneurons in the brain organoid or the primary brain slice.

A human brain organoid (e.g., a human cortical organoid) can be generated using any known method, including methods described in, e.g., U.S. Patent Publication No. 2020/0291352 and U.S. Pat. No. 10,087,417. For example, a human brain organoid can be generated by: (i) inducing a neural fate in a pluripotent stem cell suspension culture or a three-dimensional (3D) aggregation culture, to provide a spheroid of neural progenitor cells; (ii) differentiating the neural progenitor cells in the spheroid to differentiate into cortical organoids; and (iii) culturing the cortical organoids under conditions permissive for cell fusion while maintaining for an extended period of time in neural medium in the absence of growth factors. As another example, a human cortical organoid can be generated by: (i) inducing a neural fate in a pluripotent stem cell suspension or 3D aggregation culture, to provide a spheroid of neural progenitor cells; (ii) differentiating the neural progenitor cells in the spheroid to differentiate into forebrain spheroids; and (iii) differentiating the spheroid for an extended period of time in neural medium. The resulting organoid is an integrated cortical structure comprising interacting GABAergic and glutamatergic neurons. The human brain organoid (human cortical organoid) contains one or more of one or more of glial cells, neuro-epithelial cells, and oligodendrocytes. The pluripotent stem cells can be induced pluripotent stem cells (iPSCs) or human embryonic stem cells (hESCs).

A human cortical organoid suitable for use herein is a three-dimensional microphysiological system that comprises functionally-integrated excitatory glutamatergic and GABAergic neurons as well as non-neuronal cells. The human brain organoid can be generated by the directed differentiation of subdomains of the forebrain that functionally interact in development.

Mouse primary neuronal progenitors suitable for use in a method of the present disclosure include medial ganglionic eminence neuronal progenitors (MGE progenitors). MGE progenitors can be obtained using any known method. For example, MGE progenitors are obtained from mouse embryonic brains (e.g., embryonic day E12.2-14.5). See, e.g., Chen et al. (2017) Sci. Reports 7:45656; and Hsieh and Baraban (2017) eNeuro 4:e0359.

Mouse primary neuronal progenitors are introduced into the human brain organoid, thereby generating a chimeric brain organoid (e.g., a chimeric cortical organoid), and the chimeric brain organoid is cultured in vitro in a culture medium for a period of time of at least 2 days. To generate a chimeric brain organoid (e.g., chimeric cortical organoid), from about 10 to about 10³ mouse primary neuronal progenitors (e.g., MGE-progenitors) are introduced into a human brain organoid (e.g., chimeric cortical organoid). For example, to generate a chimeric cortical organoid, from about 10 to about 50, from about 50 to about 10², from about 10² to about 2×10², from about 2×10² to about 5×10², from about 5×10² to about 7×10², from about 7×10² to about 10³, from about 10³ to about 5×10³, from about 5×10³ to about 10⁴, from about 10⁴ to about 5×10⁴, from about 5×10⁴ to about 10⁵, from about 10⁵ to about 5×10⁵, from about 5×10⁵ to about 10⁶, or more than 10⁶, mouse primary neuronal progenitors (e.g., MGE-progenitors) are introduced into a human cortical organoid.

In some cases, mouse primary neuronal progenitors (e.g., MGE-progenitors) that are introduced into a human brain organoid (e.g., human cortical organoid) are wild-type (i.e., not genetically modified by human intervention). In some cases, mouse primary neuronal progenitors (e.g., MGE-progenitors) that are introduced into a human cortical organoid are genetically modified. Where the mouse primary neuronal progenitors (e.g., MGE-progenitors) are genetically modified, the genetic modification can derive from the mouse from which the mouse primary neuronal progenitors (e.g., MGE-progenitors) are obtained. For example, the mouse from which the mouse primary neuronal progenitors (e.g., MGE-progenitors) are obtained can include one or more genetic modifications that introduce one or more heterologous nucleic acids into the mouse primary neuronal progenitors (e.g., MGE-progenitors). Alternatively, mouse primary neuronal progenitors (e.g., MGE-progenitors) can be genetically modified in vitro once they are obtained from a mouse embryo.

Genetic modifications of interest include knock-ins and knock-downs. For example, genes that can be knocked down (e.g., rendered non-functional; reduced in gene expression; or deleted) include PVALB, SST, MEF2C, GAD1, GAD2, DLX5, DLX6, NKX2-1, MTOR, TSC1, TSC2, CACNA1A, CACNA1H, CACN4B, CACNA1C, SHANK3, SHANK1, SHANK2, NKX6-1, CIT, MECP2, PTEN, MAP2, RYK, SHH, BMP4, BMP7, AUTS2, DCX, CHD8, PLCXD3, ERBB4, BCAN, GPHN, DLG4, SLC32A1, SLC6A1, GABRA1, GABRA2, GABRA3, GABRA4, GABRA5, GABRA6, GABRB1, GABRB2, GABRB3, GABRG1, GABRG2, GABRG3, GABRD, GABRE, GABRP, GABRQ, GABRR1, GABRR2, GABRR3, MAFA, MAFB, SATB1, DPF1, SSBP2, CARHSP1, CUX2, ZEB2, PBX3, POU3F4, CXCR4, CXCR7, NXPH1, NRP1, NETO1, CHL1, DSCAML1, NLGN2, GRIA1, GRIA2, LAMP5, KCC2, CACNB4, SEZ6L2, DPP6, CCK, CCKAR, CENTG2, DTNA, NR2F2, NR2F1, SOX6, SOX2, KCNC2, KCNK2, SCN1A, ABAT, CACNG2, NCAM1, EPHA3, EPHB5, ARX, FOS, ACKR3, MEIS1, CITED2, HDAC11, DNMT3A, DNMT3B, DNMT1, SIRT1, NPAS3, NPAS1, MAP3K, MAP2K, MAPK, EGF, EGFR, GRB2, NTRK1, NTRK2, NTRK3, FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, FGF23, FGFR1, FGFR2, FGFR3, FGFR4, LGALS1, RBP4, SYT2, STAC2, LGI2, KCNAB3, SP8, COX6A2, CTHRC1, ETL4, CBLN4, FAM134B, TMEM91, RAB3B, PTGS1, PPARGC1A, TCAP, CORT, ST3GAL6, PTGES2, ALDH5A1, CPLX1, TAC1, NRSN2, RYR1, RYR2, THY1, BCL11B, RPTOR, DEPTOR, PKC, PRKCA, OTX2, BDNF, GDNF, LHX6, LHX8, LDB1, LMO1, LMO3, ISLET1, FZD5, GLI2, BMPER, PGC1A, DLX1, DLX2, MET, CNTNAP2, FOXG1, NRG1, DTNBP1, FMR1, NRL4X, NOVA1, NRL3, CDKL5, ASCL1, and OSTN. In some cases, a gene that is knocked down (e.g., rendered non-functional; reduced in gene expression; or deleted) is selected from: PVALB, SST, MEF2C, GAD2, DLX5, DLX6, NKX2-1, MTOR, TSC1, TSC2, MECP2, PTEN, RYK, CHD8, ERBB4, MAKA, SCN1A, EGFR, SYT2, GLI2, and LHX6.

In some cases, a gene product (e.g., mRNA or polypeptide) of a gene is increased, where the gene product is a product of any one of the following genes: PVALB, SST, MEF2C, GAD1, GAD2, DLX5, DLX6, NKX2-1, MTOR, TSC1, TSC2, CACNA1A, CACNA1H, CACN4B, CACNA1C, SHANK3, SHANK1, SHANK2, NKX6-1, CIT, MECP2, PTEN, MAP2, RYK, SHH, BMP4, BMP7, AUTS2, DCX, CHD8, PLCXD3, ERBB4, BCAN, GPHN, DLG4, SLC32A1, SLC6A1, GABRA1, GABRA2, GABRA3, GABRA4, GABRA5, GABRA6, GABRB1, GABRB2, GABRB3, GABRG1, GABRG2, GABRG3, GABRD, GABRE, GABRP, GABRQ, GABRR1, GABRR2, GABRR3, MAFA, MAFB, SATB1, DPF1, SSBP2, CARHSP1, CUX2, ZEB2, PBX3, POU3F4, CXCR4, CXCR7, NXPH1, NRP1, NETO1, CHL1, DSCAML1, NLGN2, GRIA1, GRIA2, LAMP5, KCC2, CACNB4, SEZ6L2, DPP6, CCK, CCKAR, CENTG2, DTNA, NR2F2, NR2F1, SOX6, SOX2, KCNC2, KCNK2, SCN1A, ABAT, CACNG2, NCAM1, EPHA3, EPHB5, ARX, FOS, ACKR3, MEIS1, CITED2, HDAC11, DNMT3A, DNMT3B, DNMT1, SIRT1, NPAS3, NPAS1, MAP3K, MAP2K, MAPK, EGF, EGFR, GRB2, NTRK1, NTRK2, NTRK3, FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, FGF23, FGFR1, FGFR2, FGFR3, FGFR4, LGALS1, RBP4, SYT2, STAC2, LGI2, KCNAB3, SP8, COX6A2, CTHRC1, ETL4, CBLN4, FAM134B, TMEM91, RAB3B, PTGS1, PPARGC1A, TCAP, CORT, ST3GAL6, PTGES2, ALDH5A1, CPLX1, TAC1, NRSN2, RYR1, RYR2, THY1, BCL11B, RPTOR, DEPTOR, PKC, PRKCA, OTX2, BDNF, GDNF, LHX6, LHX8, LDB1, LMO1, LMO3, ISLET1, FZD5, GLI2, BMPER, PGC1A, DLX1, DLX2, MET, CNTNAP2, FOXG1, NRG1, DTNBP1, FMR1, NRL4X, NOVA1, NRL3, CDKL5, ASCL1, and OSTN. In some cases, a gene product (e.g., mRNA or polypeptide) of a gene is increased, where the gene product is a product of a gene selected from: selected from: PVALB, SST, MEF2C, GAD2, DLX5, DLX6, NKX2-1, MTOR, TSC1, TSC2, MECP2, PTEN, RYK, CHD8, ERBB4, MAKA, SCN1A, EGFR, SYT2, GLI2, and LHX6.

In some cases, a mouse primary neuronal progenitor (e.g., a MGE-progenitor) is genetically modified with a heterologous nucleic acid comprising a nucleotide sequence encoding a mutated version of a gene product of one of the following genes: PVALB, SST, MEF2C, GAD1, GAD2, DLX5, DLX6, NKX2-1, MTOR, TSC1, TSC2, CACNA1A, CACNA1H, CACN4B, CACNA1C, SHANK3, SHANK1, SHANK2, NKX6-1, CIT, MECP2, PTEN, MAP2, RYK, SHH, BMP4, BMP7, AUTS2, DCX, CHD8, PLCXD3, ERBB4, BCAN, GPHN, DLG4, SLC32A1, SLC6A1, GABRA1, GABRA2, GABRA3, GABRA4, GABRA5, GABRA6, GABRB1, GABRB2, GABRB3, GABRG1, GABRG2, GABRG3, GABRD, GABRE, GABRP, GABRQ, GABRR1, GABRR2, GABRR3, MAFA, MAFB, SATB1, DPF1, SSBP2, CARHSP1, CUX2, ZEB2, PBX3, POU3F4, CXCR4, CXCR7, NXPH1, NRP1, NETO1, CHL1, DSCAML1, NLGN2, GRIA1, GRIA2, LAMP5, KCC2, CACNB4, SEZ6L2, DPP6, CCK, CCKAR, CENTG2, DTNA, NR2F2, NR2F1, SOX6, SOX2, KCNC2, KCNK2, SCN1A, ABAT, CACNG2, NCAM1, EPHA3, EPHB5, ARX, FOS, ACKR3, MEIS1, CITED2, HDAC11, DNMT3A, DNMT3B, DNMT1, SIRT1, NPAS3, NPAS1, MAP3K, MAP2K, MAPK, EGF, EGFR, GRB2, NTRK1, NTRK2, NTRK3, FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, FGF23, FGFR1, FGFR2, FGFR3, FGFR4, LGALS1, RBP4, SYT2, STAC2, LGI2, KCNAB3, SP8, COX6A2, CTHRC1, ETL4, CBLN4, FAM134B, TMEM91, RAB3B, PTGS1, PPARGC1A, TCAP, CORT, ST3GAL6, PTGES2, ALDH5A1, CPLX1, TAC1, NRSN2, RYR1, RYR2, THY1, BCL11B, RPTOR, DEPTOR, PKC, PRKCA, OTX2, BDNF, GDNF, LHX6, LHX8, LDB1, LMO1, LMO3, ISLET1, FZD5, GLI2, BMPER, PGC1A, DLX1, DLX2, MET, CNTNAP2, FOXG1, NRG1, DTNBP1, FMR1, NRL4X, NOVA1, NRL3, CDKL5, ASCL1, and OSTN. In some cases, a mouse primary neuronal progenitor (e.g., an MGE-progenitor) is genetically modified with a heterologous nucleic acid comprising a nucleotide sequence encoding a mutated version of a gene product of a gene selected from: selected from: PVALB, SST, MEF2C, GAD2, DLX5, DLX6, NKX2-1, MTOR, TSC1, TSC2, MECP2, PTEN, RYK, CHD8, ERBB4, MAKA, SCN1A, EGFR, SYT2, GLI2, and LHX6. Mutated versions that result in a disease or disorder can be used to genetically modified a mouse primary neuronal progenitor (e.g., an MGE-progenitor).

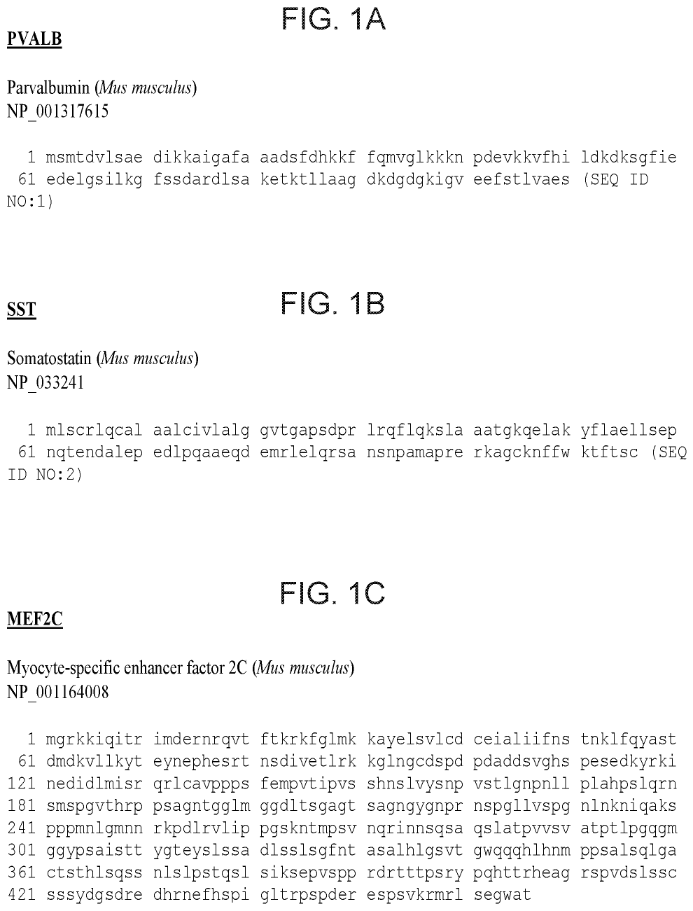

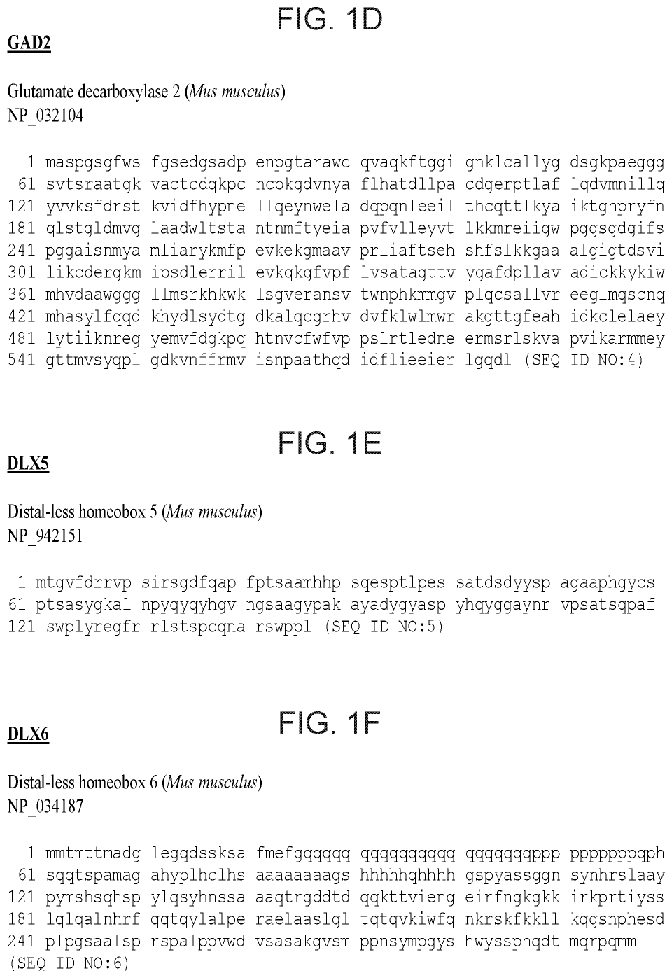

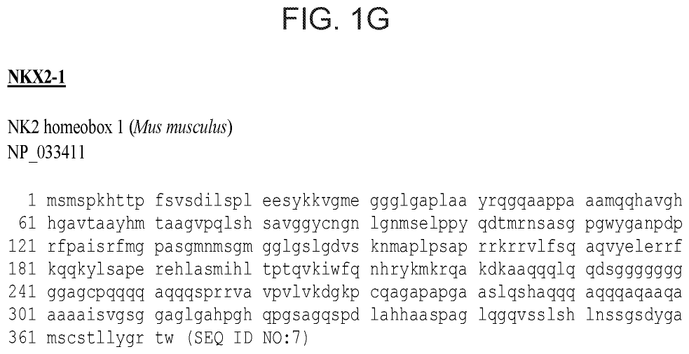

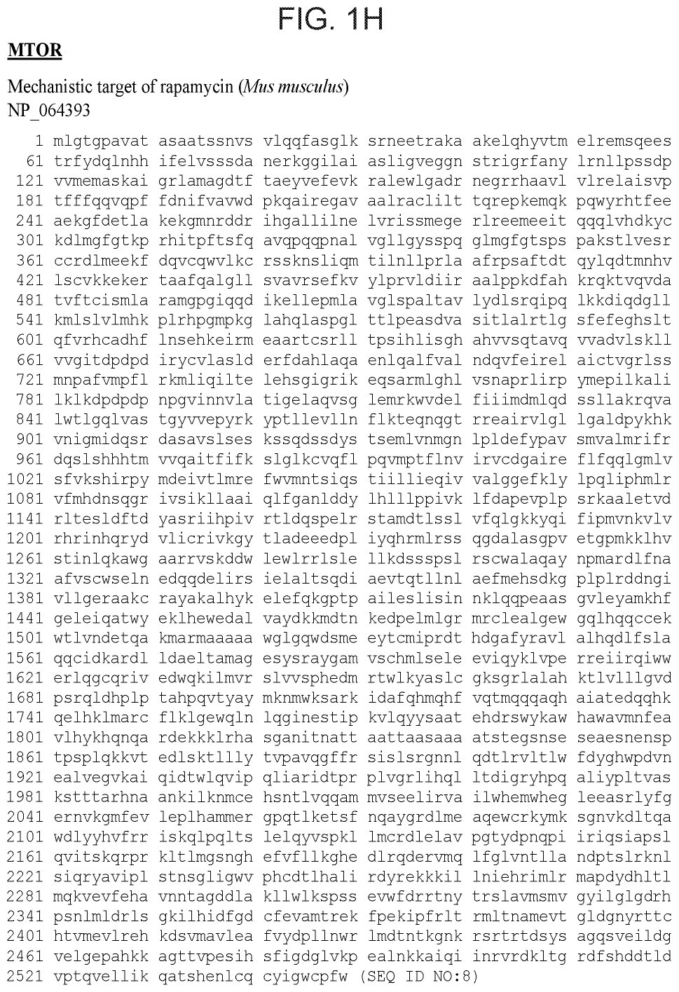

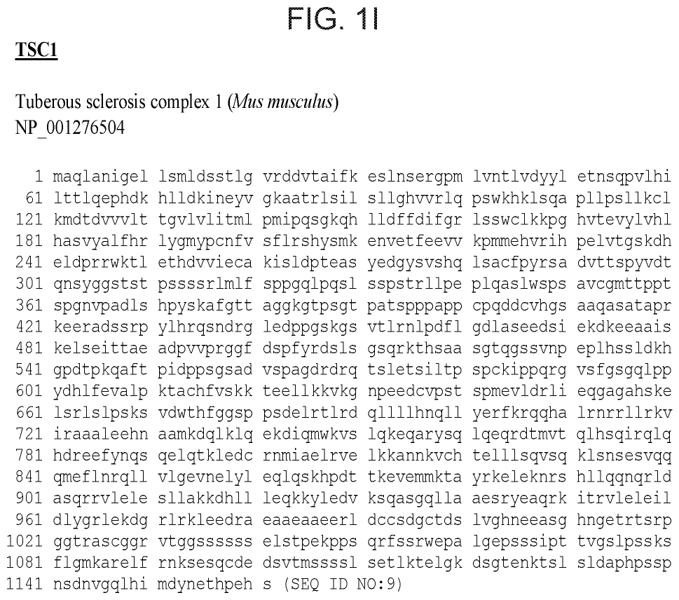

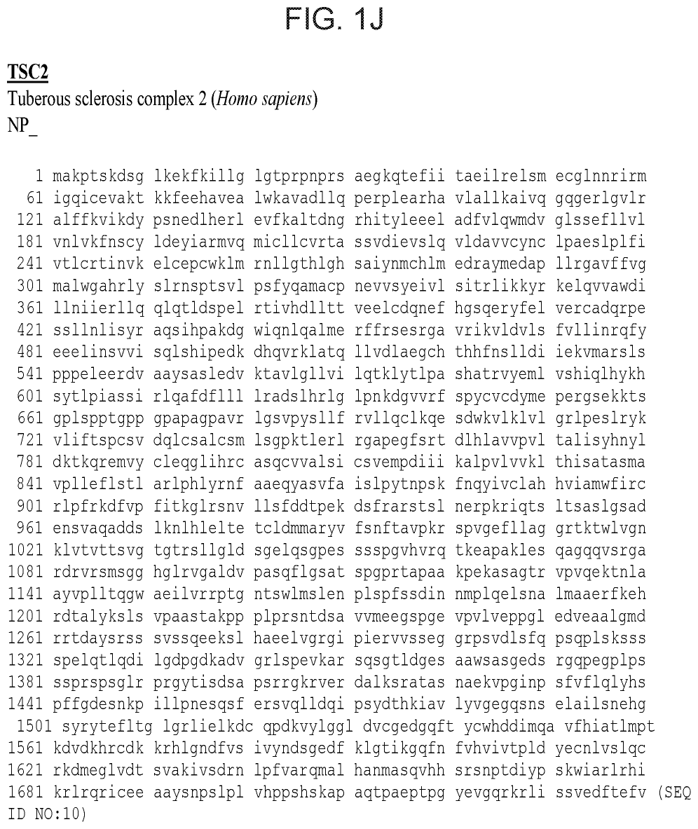

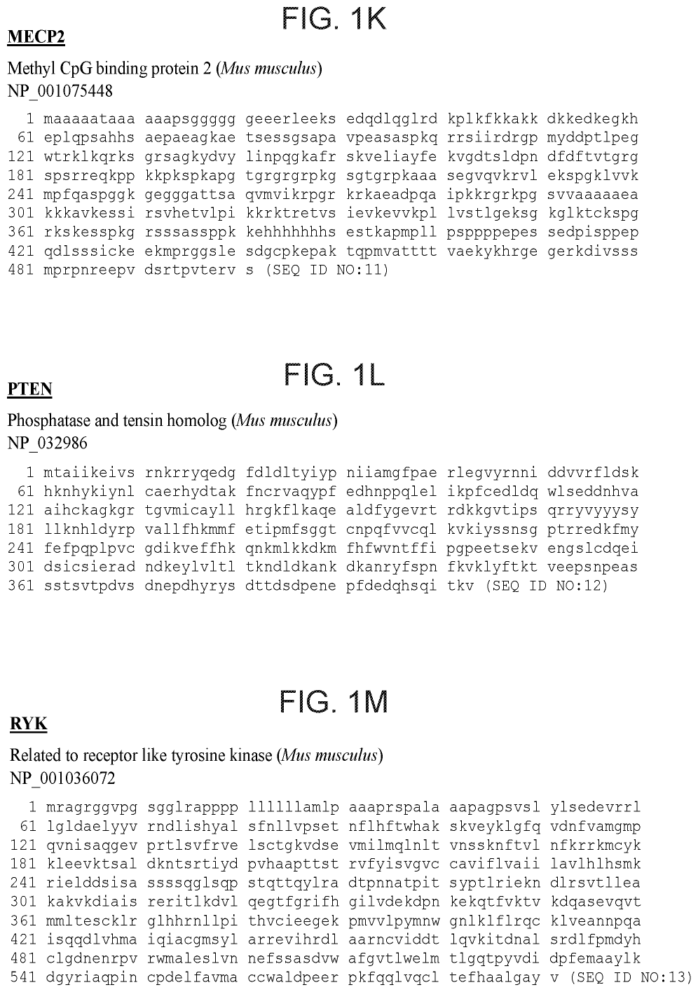

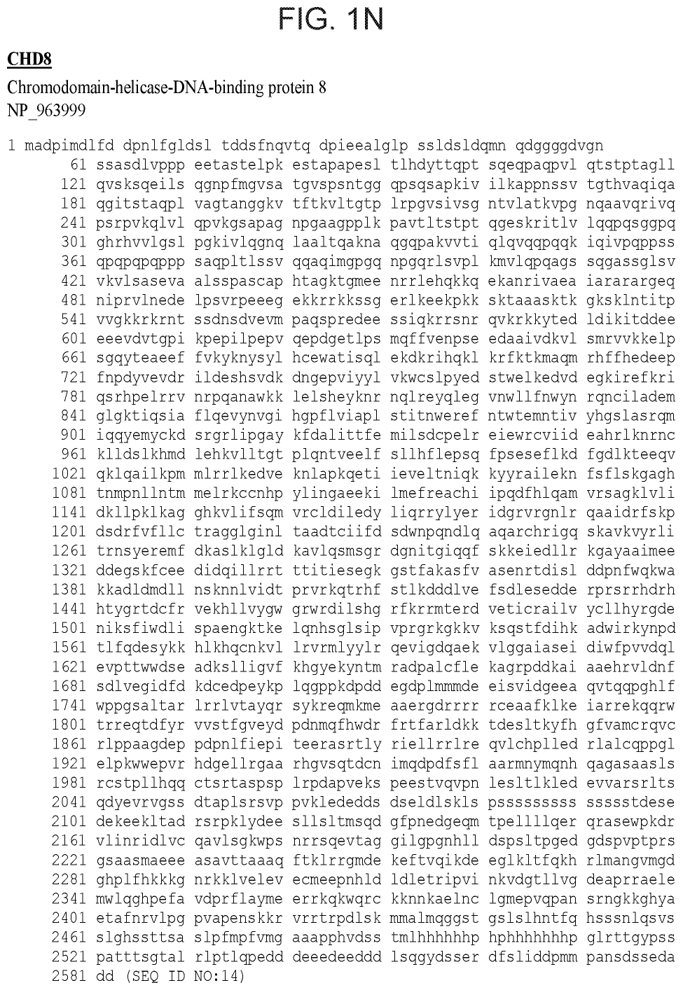

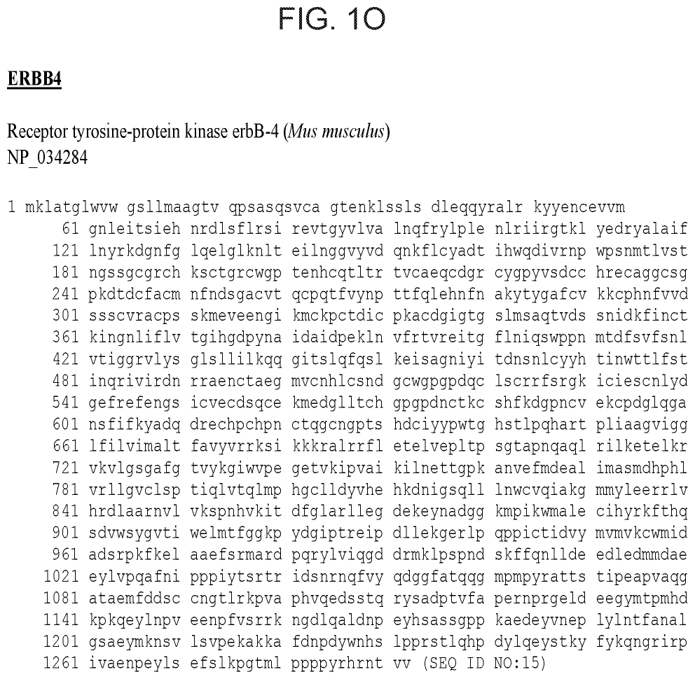

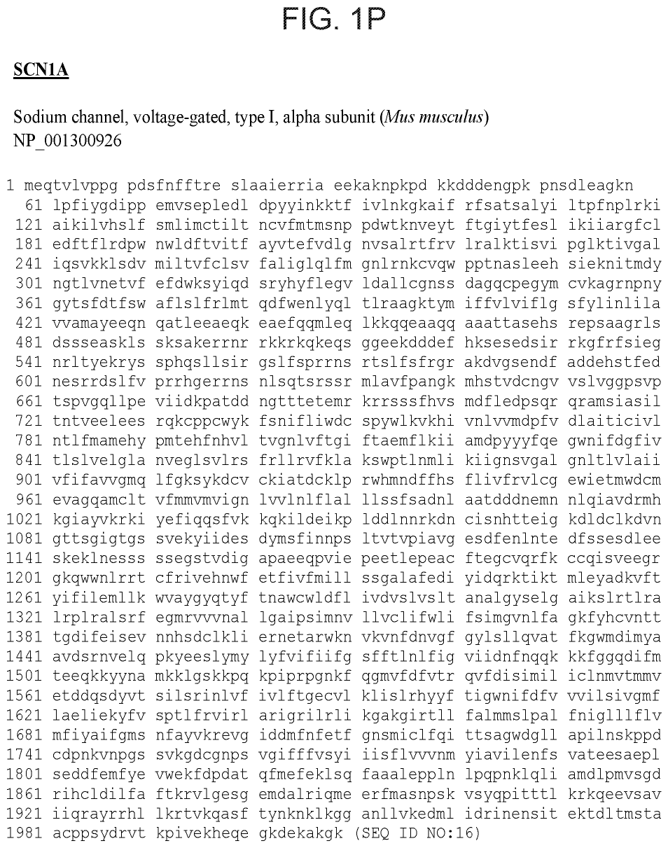

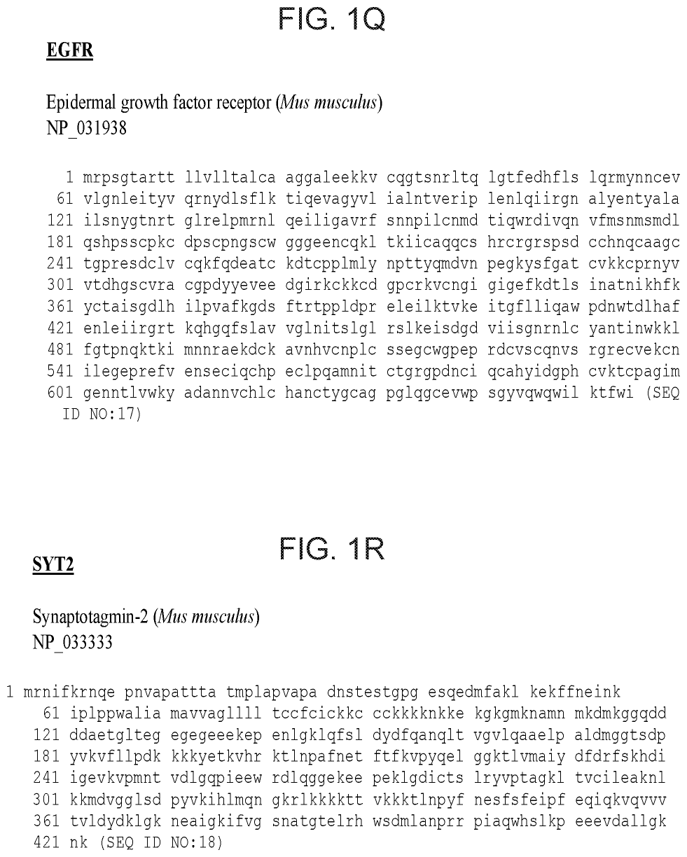

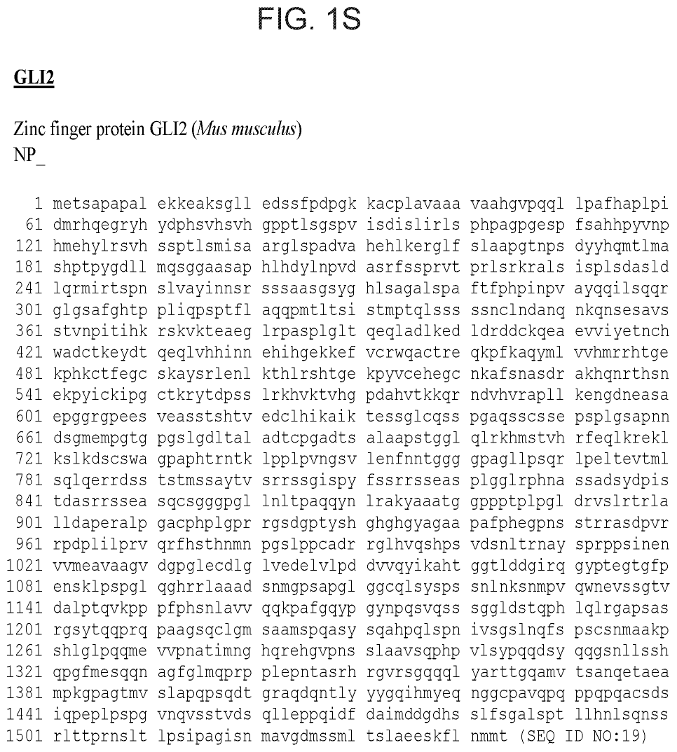

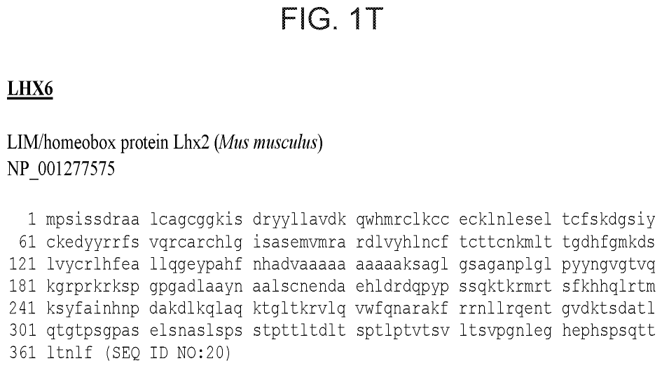

A target nucleic acid that is a target of a genetic modification can comprise a nucleotide sequence encoding a polypeptide having at least 80%, at least 85%, at least 90%, at least 95%, at least 98%, at least 99%, or 100%, amino acid sequence identity with any one of the amino acid sequences depicted in FIG. 1A-1T, where the polypeptides are PVALB, SST, MEF2C, GAD2, DLX5, DLX6, NKX2-1, MTOR, TSC1, TSC2, MECP2, PTEN, RYK, CHD8, ERBB4, SCN1A, EGFR, SYT2, GLI2, and LHX6.

Disease-associated or disease-causing mutations can be generated in a mouse primary neuronal progenitor (e.g., an MGE-progenitor) through targeted genetic manipulation (CRISPR/Cas9, etc.). Conditions of neurodevelopmental and neuropsychiatric disorders and neural diseases that have strong genetic components or are directly caused by genetic or genomic alterations can be modeled with a chimeric organoid (e.g., chimeric cortical organoid) of the present disclosure. Genetic alterations include for example: point mutations in genes such as NLGN1/3/4, NRXN1/4, SHANK1/2/3, GRIN2B/A, FMR1, or CHD8 that represent risk alleles for autism spectrum disorders; point mutations in or deletions of genes such as CACNA1C, CACNB2, NLGN4X, LAMA2, DPYD, TRRAP, MMP16, NRXN1 or NIPAL3 that are associated with schizophrenia or autism spectrum disorders (ASD); a triplet expansion in the HTT gene that cause to Huntington's disease (HD); monoallelic mutations in genes such as SNCA, LRRK2 and biallelic mutations in genes such as PINK1, DJ-1, or ATP13A2 that predispose to Parkinson disease (PD); single nucleotide polymorphisms (SNPs) in genes such as ApoE, APP, and PSEN1/2 that confer risks for developing Alzheimer's disease (AD) and other forms of dementia; single nucleotide polymorphisms (SNPs) in genes such as CACNA1C, CACNB3, ODZ4, ANK3 that are associated with bipolar disease (BP); Angelman (UBE3A); Rett (MEPC2); and Tuberous sclerosis (TSC1/2). Genomic alterations include copy number variations (CNVs) such as deletions or duplications of 1q21.1, 7q11.23, 15q11.2, 15q13.3, 22q11.2 or 16p11.2, 16p13.3 that are associated with ASD, schizophrenia, intellectual disability, epilepsy, etc.; trisomy 21 and Down Syndrome, Fragile X syndrome caused by alteration of the FMR1 gene. Any number of neurodevelopment disorders with a defined genetic etiology can be additionally modeled by introducing mutations in or completely removing disease-relevant gene(s) using genome editing.

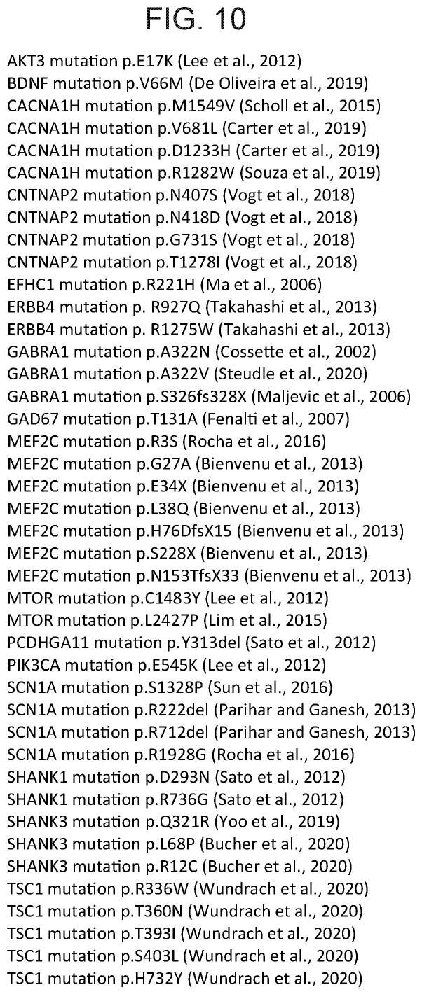

Non-limiting examples of such mutations are provided in FIG. 10. The mutations listed in FIG. 10 have been reported, as indicated by reference in FIG. 10 to publications. The publications listed in FIG. 10 are as follows: 1) Bienvenu et al. (2013). Refining the phenotype associated with MEF2C point mutations. Neurogenetics 14:71-75: 10.1007/s10048-012-0344-7; 2) Bucher et al. (2020). Autism associated SHANK3 missense point mutations impact conformational fluctuations and protein turnover at synapses. Biorxiv: 424970. DOI: 10.1101/2020.12.31.424970; 3) Carter et al. Compound heterozygous CACNA1H mutations associated with severe congenital amyotrophy. Channels 13 (1): 153-161. DOI: 10.1080/19336950.2019.1614415; 4) Cossette et al. (2002). Mutation of GABRA1 in an autosomal dominant form of juvenile myoclonic epilepsy. Nature Genetics 31:184-189. DOI: 10.1038/ng885; 5) De Oliveira et al. (2019). In silico analysis of the V66M variant of human BDNF in psychiatric disorders: An approach to precision medicine. PLOS ONE: 0215508. DOI: 10.1371/journal.pone.0215508.; 6) Fenalti et al. (2007). GABA production by glutamic acid decarboxylase is regulated by a dynamic catalytic loop. Nature Structural and Molecular Biology 14:280-286. DOI: 10.1038/nsmb1228; 7) Lee et al. (2012). De novo somatic mutations in components of the PI3K-AKT3-mTOR pathway cause hemimegalencephaly. Nature Genetics 44: 941-945. DOI: 10.1038/ng.2329; 8) Lim et al. (2015). Brain somatic mutations in MTOR cause focal cortical dysplasia type II leading to intractable epilepsy. Nature Medicine 21: 395-400. DOI: 10.1038/nm.3824; 9) Ma et al. (2006). Mutations in the GABRA1 and EFHC1 genes are rare in familial juvenile myoclonic epilepsy. Epilepsy Research 71 (2-3): 129-134. DOI: j.eplepsyres.2006.06.001; 10) Maljevic et al. (2006). A mutation in the GABAA receptor α1-subunit is associated with absence epilepsy. Annals of Neurology 59 (6): 983-987. DOI: 10.1002/ana.20874; 11) Parihar and Ganesh (2013). The SCN1A gene variants and epileptic encephalopathies. Journal of Human Genetics 58:573-580. DOI: 10.1038/jhg.2013.77; 12) Rocha et al. (2016). MEF2C haploinsufficiency syndrome: Report of a new MEF2C mutation and review. European Journal of Medical Genetics 59 (9): 478-482. DOI: 10.1016/j.ejmg.2016.05.017; 13) Sato et al. (2012). SHANK1 Deletions in Males with Autism Spectrum Disorder. American Journal of Human Genetics 90 (5): 879-887. DOI: 10.1016/j.ajhg.2012.03.017; 14) Scholl et al. (2015). Recurrent gain of function mutation in calcium channel CACNA1H causes early-onset hypertension with primary aldosteronism. eLife 4: c06315. DOI: 10.7554/eLife.06315; 15) Souza et al. (2019). Pathogenic Cav3.2 channel mutation in a child with primary generalized epilepsy. Molecular Brain 12: 86. DOI: 10.1186/s13041-019-0509-5; 16) Steudle et al. (2020). A novel de novo variant of GABRA1 causes increased sensitivity for GABA in vitro. Scientific Reports 10: 2379. DOI: 10.1038/s41598-020-59323-6; 17) Sun et al. (2016). A deleterious Nav1.1 mutation selectively impairs telencephalic inhibitory neurons derived from Dravet Syndrome patients. eLife 5: e13073 DOI: 10.7554/eLife.13073; 18) Takahashi et al. (2013). ERBB4 Mutations that Disrupt the Neuregulin-ErbB4 Pathway Cause Amyotrophic Lateral Sclerosis Type 19. American Journal of Human Genetics 93 (5): 900-905. DOI: 10.1016/j.ajhg.2013.09.008; 19) Vogt et al. (2018). Mouse Cntnap2 and Human CNTNAP2 ASD Alleles Cell Autonomously Regulate PV+ Cortical Interneurons. Cerebral Cortex 28 (11): 3868-3879. DOI: 10.1093/cercor/bhx248; 20) Wundrach et al. (2020). A human TSC1 variant screening platform in GABAergic cortical interneurons for genotype to phenotype assessments. Frontiers in Molecular Neuroscience. 13: 573409. DOI: 10.3389/fnmol.2020.573409; 21) Yoo et al. (2019). Shank3 Mice Carrying the Human Q321R Mutation Display Enhanced Self-Grooming, Abnormal Electroencephalogram Patterns, and Suppressed Neuronal Excitability and Seizure Susceptibility. Frontiers in Molecular Neuroscience. Frontiers in Molecular Neuroscience 12: 155. DOI: 10.3389/fnmol.2019.00155

Methods of genetically modifying a cell are known, and any such method can be used. For example, a CRISPR/Cas system (a CRISPR/Cas polypeptide and a guide RNA) can be used to delete all or a portion of a target nucleic acid of interest, or to introduce one or more mutations (single nucleotide substitutions, insertions, and the like) into a target nucleic acid of interest, or to replace a target nucleic acid of interest with a modified version of the nucleic acid. A CRISPR/Cas system (a CRISPR/Cas polypeptide and a guide RNA) can also be used to decrease transcription of a target nucleic acid of interest.

Mouse primary neuronal progenitors (e.g., MGE-progenitors) are introduced into a human brain organoid (e.g., a human cortical organoid), thereby generating a chimeric brain organoid (e.g., a chimeric cortical organoid), and the chimeric brain organoid (e.g., the chimeric cortical organoid) is cultured in vitro in a culture medium for a period of time of at least 2 days, e.g., for a period of time of from about 2 days to about 180 days (e.g., from about 2 days to 14 days, from about 14 days to about 30 days, from about 1 month to about 2 months, from about 2 months to about 3 months, from about 3 months to about 4 months, from about 4 months to about 5 months, or from about 5 months to about 6 months, or longer than 6 months). For example, the chimeric brain organoid (e.g., the chimeric cortical organoid) is cultured in vitro in a culture medium for a period of time of from about 2 days to about 4 days, from about 4 days to about 7 days, from about 7 days to about 10 days, from about 10 days to about 14 days, from about 14 days to about 20 days, from about 20 days to about 25 days, from about 25 days to about 28 days, from about 28 days to about 30 days, from about 1 month to about 2 months, from about 2 months to about 3 months, from about 3 months to about 4 months, from about 4 months to about 5 months, or from about 5 months to about 6 months, or longer than 6 months.

Culturing the chimeric brain organoid in culture in vitro generates a chimeric organoid that comprises an enriched population of parvalbumin-positive interneurons. Parvalbumin-positive (PV⁺) interneurons are GABAergic interneurons that primarily form fast synapses around the somatic regions of pyramidal cells. PV⁺ interneurons (e.g., an enriched population of mouse PV⁺ interneurons) are generated within 2 days following introduction of mouse primary neuronal progenitors (e.g., MGE-progenitors) into a human brain organoid (e.g., human cortical organoid). As used herein, the term “mouse PV⁺ interneurons” refers to PV⁺ interneurons that are differentiated from mouse primary neuronal progenitors (e.g., MGE-progenitors) following introduction of the mouse primary neuronal progenitors (e.g., MGE-progenitors) into a human brain organoid (e.g., human cortical organoid). The mouse PV⁺ interneurons so generated are of mouse origin, but may be genetically modified with one or more heterologous nucleic acids such that they are not, strictly speaking, “mouse” PV⁺ interneurons; nevertheless, due to their origin in the mouse, an enriched population of PV⁺ interneurons generated using a method of the present disclosure is referred to as “mouse PV⁺ interneurons.” The mouse PV⁺ interneurons generated using a method of the present disclosure increase in number during culture of a chimeric brain organoid (e.g., chimeric cortical organoid), and thus generate a population of “enriched PV⁺ interneurons.”

At least 50% of the mouse primary neuronal progenitors (e.g., MGE-progenitors) introduced into a human brain organoid (e.g., human cortical organoid) differentiate into PV⁺ interneurons following culture of the chimeric organoid (e.g., chimeric cortical organoid) for the above-noted period of time. For example, after a period of from about 5 days to about 30 days in culture, at least 50%, at least 60%, at least 70%, at least 75%, at least 80%, at least 85%, at least 90%, or at least 95%, of the mouse primary neuronal progenitors (e.g., MGE-progenitors) differentiate into PV⁺ interneurons. For example, after a period of about 28 days in culture, at least 50%, at least 60%, at least 70%, at least 75%, at least 80%, at least 85%, at least 90%, or at least 95%, of the mouse primary neuronal progenitors (e.g., MGE-progenitors) differentiate into PV⁺ interneurons.

A method of the present disclosure generates an enriched population of PV⁺ interneurons. The enriched population of PV⁺ interneurons can comprise from about 10⁴ to about 10⁸ PV⁺ interneurons. For example, the enriched population of PV⁺ interneurons can comprise from about 10³ to about 10⁴, from about 10⁴ to about 10⁵, from about 10⁵ to about 5×10⁵, from about 5×10⁵ to about 10⁶, from about 10⁶ to about 5×10⁶, from about ×10⁶ to about 10⁷, or from about 10⁷ to about 10⁸ PV⁺ interneurons. At least 75%, at least 80%, at least 85%, at least 90%, at least 95%, or at least 98%, of enriched population are PV⁺ interneurons.

A method of the present disclosure can generate an organoid that comprises perineuronal nets (PNNs). PNNs are specialized extracellular matrix complexes that preferentially surround PV⁺ interneurons. Abnormalities in PNNs are involved in a variety of disorders, including Alzheimer's disease (AD) and schizophrenia. PNNs require PV⁺ interneurons for formation. Carceller et al. (2020) J. Neurosci. 40:5008; Wen et al. (2018) Frontiers Mol. Neurosci. 11:270. Thus, in some cases, PV⁺ interneurons generated according to a method of the present disclosure are present in PNNs. An organoid of the present disclosure can thus serve as an in vitro model of PNNs and their involvement in disorders such as AD and schizophrenia.

A method of the present disclosure can generate a chimeric organoid (e.g., a brain organoid comprising tissue of human origin comprising an enriched population of PV⁺ interneurons of mouse origin. A method of the present disclosure can generate a chimeric cortical organoid (e.g., a cortical organoid comprising tissue of human origin comprising an enriched population of PV⁺ interneurons of mouse origin.

In some cases, a method of the present disclosure further comprises isolating the enriched PV⁺ interneurons from the chimeric organoid (e.g., chimeric cortical organoid).

### Chimeric Organoids

The present disclosure provides a chimeric organoid (e.g., a chimeric cortical organoid) comprising an enriched population of parvalbumin-positive interneurons. A chimeric organoid of the present disclosure comprises: a) a human brain organoid; and b) an enriched population of mouse PV⁺ interneurons. For example, a chimeric cortical organoid of the present disclosure comprises: a) a human cortical organoid; and b) an enriched population of mouse PV⁺ interneurons.

The enriched population of PV⁺ interneurons in a chimeric organoid (e.g., a chimeric cortical organoid) of the present disclosure can comprise from about 10⁴ to about 10⁸ PV⁺ interneurons. For example, the enriched population of PV⁺ interneurons can comprise from about 10³ to about 10⁴, from about 10⁴ to about 10⁵, from about 10⁵ to about 5×10, from about 5×10⁵ to about 10⁶, from about 10⁶ to about 5×10⁶, from about 5×10⁶ to about 10⁷, or from about 10⁷ to about 10⁸ PV⁺ interneurons. At least 75%, at least 80%, at least 85%, at least 90%, at least 95%, or at least 98%, of enriched population are PV⁺ interneurons.

PV⁺ interneurons present in a chimeric organoid (e.g., a chimeric cortical organoid) of the present disclosure can be present in PNNs. PNNs comprise chondroitin sulfate proteoglycans (CSPGs) (e.g., CSPGs such as tenascin, neurocan, versican, brevican, and aggrecan) and can include hyaluronic acid. The PNNs can surround the cell body and/or neurites.

The PV⁺ interneurons in an enriched population of PV⁺ interneurons present in a chimeric organoid (e.g., a chimeric cortical organoid) of the present disclosure can have a wild-type genome. Alternatively, the PV⁺ interneurons in an enriched population of PV⁺ interneurons present in a chimeric organoid (e.g., a chimeric cortical organoid) of the present disclosure can comprise one or more genetic modifications.

Genetic modifications of interest include knock-ins and knock-downs. For example, genes that can be knocked down (e.g., rendered non-functional; reduced in gene expression; or deleted) include PVALB, SST, MEF2C, GAD1, GAD2, DLX5, DLX6, NKX2-1, MTOR, TSC1, TSC2, CACNA1A, CACNA1H, CACN4B, CACNA1C, SHANK3, SHANK1, SHANK2, NKX6-1, CIT, MECP2, PTEN, MAP2, RYK, SHH, BMP4, BMP7, AUTS2, DCX, CHD8, PLCXD3, ERBB4, BCAN, GPHN, DLG4, SLC32A1, SLC6A1, GABRA1, GABRA2, GABRA3, GABRA4, GABRA5, GABRA6, GABRB1, GABRB2, GABRB3, GABRG1, GABRG2, GABRG3, GABRD, GABRE, GABRP, GABRQ, GABRR1, GABRR2, GABRR3, MAFA, MAFB, SATB1, DPF1, SSBP2, CARHSP1, CUX2, ZEB2, PBX3, POU3F4, CXCR4, CXCR7, NXPH1, NRP1, NETO1, CHL1, DSCAML1, NLGN2, GRIA1, GRIA2, LAMP5, KCC2, CACNB4, SEZ6L2, DPP6, CCK, CCKAR, CENTG2, DTNA, NR2F2, NR2F1, SOX6, SOX2, KCNC2, KCNK2, SCN1A, ABAT, CACNG2, NCAM1, EPHA3, EPHB5, ARX, FOS, ACKR3, MEIS1, CITED2, HDAC11, DNMT3A, DNMT3B, DNMT1, SIRT1, NPAS3, NPAS1, MAP3K, MAP2K, MAPK, EGF, EGFR, GRB2, NTRK1, NTRK2, NTRK3, FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, FGF23, FGFR1, FGFR2, FGFR3, FGFR4, LGALS1, RBP4, SYT2, STAC2, LGI2, KCNAB3, SP8, COX6A2, CTHRC1, ETL4, CBLN4, FAM134B, TMEM91, RAB3B, PTGS1, PPARGC1A, TCAP, CORT, ST3GAL6, PTGES2, ALDH5A1, CPLX1, TAC1, NRSN2, RYR1, RYR2, THY1, BCL11B, RPTOR, DEPTOR, PKC, PRKCA, OTX2, BDNF, GDNF, LHX6, LHX8, LDB1, LMO1, LMO3, ISLET1, FZD5, GLI2, BMPER, PGC1A, DLX1, DLX2, MET, CNTNAP2, FOXG1, NRG1, DTNBP1, FMR1, NRL4X, NOVA1, NRL3, CDKL5, ASCL1, and OSTN. In some cases, a gene that is knocked down (e.g., rendered non-functional; reduced in gene expression; or deleted) is selected from: PVALB, SST, MEF2C, GAD2, DLX5, DLX6, NKX2-1, MTOR, TSC1, TSC2, MECP2, PTEN, RYK, CHD8, ERBB4, MAKA, SCN1A, EGFR, SYT2, GLI2, and LHX6.

In some cases, a gene product (e.g., mRNA or polypeptide) of a gene is increased, where the gene product is a product of any one of the following genes: PVALB, SST, MEF2C, GAD1, GAD2, DLX5, DLX6, NKX2-1, MTOR, TSC1, TSC2, CACNA1A, CACNA1H, CACN4B, CACNA1C, SHANK3, SHANK1, SHANK2, NKX6-1, CIT, MECP2, PTEN, MAP2, RYK, SHH, BMP4, BMP7, AUTS2, DCX, CHD8, PLCXD3, ERBB4, BCAN, GPHN, DLG4, SLC32A1, SLC6A1, GABRA1, GABRA2, GABRA3, GABRA4, GABRA5, GABRA6, GABRB1, GABRB2, GABRB3, GABRG1, GABRG2, GABRG3, GABRD, GABRE, GABRP, GABRQ, GABRR1, GABRR2, GABRR3, MAFA, MAFB, SATB1, DPF1, SSBP2, CARHSP1, CUX2, ZEB2, PBX3, POU3F4, CXCR4, CXCR7, NXPH1, NRP1, NETO1, CHL1, DSCAML1, NLGN2, GRIA1, GRIA2, LAMP5, KCC2, CACNB4, SEZ6L2, DPP6, CCK, CCKAR, CENTG2, DTNA, NR2F2, NR2F1, SOX6, SOX2, KCNC2, KCNK2, SCN1A, ABAT, CACNG2, NCAM1, EPHA3, EPHB5, ARX, FOS, ACKR3, MEIS1, CITED2, HDAC11, DNMT3A, DNMT3B, DNMT1, SIRT1, NPAS3, NPAS1, MAP3K, MAP2K, MAPK, EGF, EGFR, GRB2, NTRK1, NTRK2, NTRK3, FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, FGF23, FGFR1, FGFR2, FGFR3, FGFR4, LGALS1, RBP4, SYT2, STAC2, LGI2, KCNAB3, SP8, COX6A2, CTHRC1, ETL4, CBLN4, FAM134B, TMEM91, RAB3B, PTGS1, PPARGC1A, TCAP, CORT, ST3GAL6, PTGES2, ALDH5A1, CPLX1, TAC1, NRSN2, RYR1, RYR2, THY1, BCL11B, RPTOR, DEPTOR, PKC, PRKCA, OTX2, BDNF, GDNF, LHX6, LHX8, LDB1, LMO1, LMO3, ISLET1, FZD5, GLI2, BMPER, PGC1A, DLX1, DLX2, MET, CNTNAP2, FOXG1, NRG1, DTNBP1, FMR1, NRL4X, NOVA1, NRL3, CDKL5, ASCL1, and OSTN. In some cases, a gene product (e.g., mRNA or polypeptide) of a gene is increased, where the gene product is a product of a gene selected from: selected from: PVALB, SST, MEF2C, GAD2, DLX5, DLX6, NKX2-1, MTOR, TSC1, TSC2, MECP2, PTEN, RYK, CHD8, ERBB4, MAKA, SCN1A, EGFR, SYT2, GLI2, and LHX6.

In some cases, the PV⁺ interneurons in an enriched population of PV⁺ interneurons present in a chimeric organoid (e.g., a chimeric cortical organoid) of the present disclosure have differentiated from mouse primary neuronal progenitors (e.g., a MGE-progenitors) that were genetically modified with a heterologous nucleic acid comprising a nucleotide sequence encoding a mutated version of a gene product of one of the following genes: PVALB, SST, MEF2C, GAD1, GAD2, DLX5, DLX6, NKX2-1, MTOR, TSC1, TSC2, CACNA1A, CACNA1H, CACN4B, CACNA1C, SHANK3, SHANK1, SHANK2, NKX6-1, CIT, MECP2, PTEN, MAP2, RYK, SHH, BMP4, BMP7, AUTS2, DCX, CHD8, PLCXD3, ERBB4, BCAN, GPHN, DLG4, SLC32A1, SLC6A1, GABRA1, GABRA2, GABRA3, GABRA4, GABRA5, GABRA6, GABRB1, GABRB2, GABRB3, GABRG1, GABRG2, GABRG3, GABRD, GABRE, GABRP, GABRQ, GABRR1, GABRR2, GABRR3, MAFA, MAFB, SATB1, DPF1, SSBP2, CARHSP1, CUX2, ZEB2, PBX3, POU3F4, CXCR4, CXCR7, NXPH1, NRP1, NETO1, CHL1, DSCAML1, NLGN2, GRIA1, GRIA2, LAMP5, KCC2, CACNB4, SEZ6L2, DPP6, CCK, CCKAR, CENTG2, DTNA, NR2F2, NR2F1, SOX6, SOX2, KCNC2, KCNK2, SCN1A, ABAT, CACNG2, NCAM1, EPHA3, EPHB5, ARX, FOS, ACKR3, MEIS1, CITED2, HDAC11, DNMT3A, DNMT3B, DNMT1, SIRT1, NPAS3, NPAS1, MAP3K, MAP2K, MAPK, EGF, EGFR, GRB2, NTRK1, NTRK2, NTRK3, FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, FGF23, FGFR1, FGFR2, FGFR3, FGFR4, LGALS1, RBP4, SYT2, STAC2, LGI2, KCNAB3, SP8, COX6A2, CTHRC1, ETL4, CBLN4, FAM134B, TMEM91, RAB3B, PTGS1, PPARGC1A, TCAP, CORT, ST3GAL6, PTGES2, ALDH5A1, CPLX1, TAC1, NRSN2, RYR1, RYR2, THY1, BCL11B, RPTOR, DEPTOR, PKC, PRKCA, OTX2, BDNF, GDNF, LHX6, LHX8, LDB1, LMO1, LMO3, ISLET1, FZD5, GLI2, BMPER, PGC1A, DLX1, DLX2, MET, CNTNAP2, FOXG1, NRG1, DTNBP1, FMR1, NRL4X, NOVA1, NRL3, CDKL5, ASCL1, and OSTN. In some cases, the PV⁺ interneurons in an enriched population of PV⁺ interneurons present in a chimeric organoid (e.g., a chimeric cortical organoid) of the present disclosure have differentiated from mouse primary neuronal progenitors (e.g., a MGE-progenitors) that were genetically modified with a heterologous nucleic acid comprising a nucleotide sequence encoding a mutated version of a gene product of a gene selected from: selected from: PVALB, SST, MEF2C, GAD2, DLX5, DLX6, NKX2-1, MTOR, TSC1, TSC2, MECP2, PTEN, RYK, CHD8, ERBB4, MAKA, SCN1A, EGFR, SYT2, GLI2, and LHX6. Mutated versions that result in a disease or disorder can be used to genetically modified a mouse primary neuronal progenitor (e.g., an MGE-progenitor).

A target nucleic acid that is a target of a genetic modification can comprise a nucleotide sequence encoding a polypeptide having at least 80%, at least 85%, at least 90%, at least 95%, at least 98%, at least 99%, or 100%, amino acid sequence identity with any one of the amino acid sequences depicted in FIG. 1A-1T, where the polypeptides are PVALB. SST, MEF2C, GAD2, DLX5, DLX6, NKX2-1, MTOR, TSC1, TSC2, MECP2, PTEN, RYK, CHD8, ERBB4, SCN1A, EGFR, SYT2, GLI2, and LHX6.

Disease-associated or disease-causing genotypes can be generated through targeted genetic manipulation (CRISPR/Cas9, etc.), e.g., targeted genetic manipulation of mouse primary neuronal progenitors (e.g., MGE-progenitors) that differentiate into PV⁺ interneurons. Conditions of neurodevelopmental and neuropsychiatric disorders and neural diseases that have strong genetic components or are directly caused by genetic or genomic alterations can be modeled with a chimeric organoid (e.g., a chimeric cortical organoid) of the present disclosure. Genetic alterations include for example: point mutations in genes such as NLGN1/3/4, NRXN1/4, SHANK1/2/3, GRIN2B/A, FMR1, or CHD8 that represent risk alleles for autism spectrum disorders; point mutations in or deletions of genes such as CACNA1C, CACNB2, NLGN4X, LAMA2, DPYD, TRRAP, MMP16, NRXN1 or NIPAL3 that are associated with schizophrenia or autism spectrum disorders (ASD); a triplet expansion in the HTT gene that cause to Huntington's disease (HD); monoallelic mutations in genes such as SNCA, LRRK2 and biallelic mutations in genes such as PINK1, DJ-1, or ATP13A2 that predispose to Parkinson disease (PD); single nucleotide polymorphisms (SNPs) in genes such as ApoE, APP, and PSEN1/2 that confer risks for developing Alzheimer's disease (AD) and other forms of dementia; single nucleotide polymorphisms (SNPs) in genes such as CACNA1C, CACNB3, ODZ4, ANK3 that are associated with bipolar disease (BP); Angelman (UBE3A); Rett (MEPC2); and Tuberous sclerosis (TSC1/2). Genomic alterations include copy number variations (CNVs) such as deletions or duplications of 1921.1, 7q11.23, 15q11.2, 15q13.3, 22q11.2 or 16p11.2, 16p13.3 that are associated with ASD, schizophrenia, intellectual disability, epilepsy, etc.; trisomy 21 and Down Syndrome, Fragile X syndrome caused by alteration of the FMR1 gene. Any number of neurodevelopment disorders with a defined genetic etiology can be additionally modeled by introducing mutations in or completely removing disease-relevant gene(s) using genome editing. Non-limiting examples of disease-associated mutations are provided in FIG. 10.

### Utility

A chimeric organoid (e.g., a chimeric cortical organoid) of the present disclosure is useful for drug screening and research applications. For example, various disorders can be studied using a chimeric organoid (e.g., a chimeric cortical organoid) of the present disclosure. As another example, a chimeric organoid (e.g., a chimeric cortical organoid) of the present disclosure is useful for identifying candidate agents for treating various disorders, including neurodegenerative disorders (Alzheimer's disease (AD), Parkinson's disease (PD), Huntington's disease (HD), and amyotrophic lateral sclerosis (ALS)); psychiatric conditions such as schizophrenia and other psychoses; bipolar disorders; mood disorders; intellectual disability (ID); and autism spectrum disorders (ASD).

The present disclosure provides methods of identifying agents that modulate a feature of a parvalbumin-positive interneuron. Such methods generally involve: a) contacting the population of PV⁺ interneurons (e.g., PV⁺ interneurons present in a chimeric organoid (e.g., a chimeric cortical organoid) of the present disclosure) with a test agent; and b) determining the effect of the test agent on a feature of the PV⁺ interneurons. Features include, e.g., viability, physiology, morphology, connectivity with other neurons, and gene expression. In some cases, modulation of PNNs is assayed. For example, in some cases, the integrity of a PNN is assayed using genetic reporters of extracellular matrix proteins, mass spectrometry or molecules such as Wisteria Fluoribunda Agglutinin (WFA) or other agglutinins.

Usually at least one control is included, for example a negative control and a positive control. Culture of cells is typically performed in a sterile environment, for example, at 37° C. in an incubator containing a humidified 60-95% air/5-40% CO₂ atmosphere. Cell culture may be carried out in nutrient mixtures containing undefined biological fluids such as fetal calf serum, or media which is fully defined and serum free. The effect of a test is assessed by monitoring one or more output parameters, including morphological changes, functional changes (e.g., physiological changes), and genetic changes (e.g., changes in gene expression).

Physiological features can be assayed using, e.g., current-clamp recording, voltage-clamp recording, multielectrode arrays, optical voltage and activity indicators, and the like.

Live imaging of PV⁺ interneurons can be performed, e.g., where the PV⁺ interneurons express a detectable marker. Calcium sensitive dyes can be used, e.g. Fura-2 calcium imaging; Fluo-4 calcium imaging, GCaMP6 calcium imaging, GCaMP7 calcium imaging, voltage imaging using voltage indicators such as voltage-sensitive dyes (e.g. di-4-ANEPPS, di-8-ANEPPS, and RH237) and/or genetically-encoded voltage indicators (e.g. ASAP1/2, Archer, QuasAr1/2/3) can be used on the intact chimeric organoid (e.g., chimeric cortical organoid) or on PV⁺ interneurons isolated from the chimeric organoid (e.g., chimeric cortical organoid).

Methods of analysis at the single cell level are also of interest, e.g. as described above: live imaging (including confocal or light-sheet microscopy), single cell gene expression or single cell RNA sequencing, calcium imaging, immunocytochemistry, patch-clamping, flow cytometry and the like. Various parameters can be measured to determine the effect of a test agent on PV⁺ interneurons in the intact chimeric organoid. or PV⁺ interneurons isolated from the chimeric organoid (e.g., chimeric cortical organoid).

Parameters are quantifiable components of cells, particularly components that can be accurately measured, desirably in a high throughput system. A parameter can also be any cell component or cell product including cell surface determinant, receptor, protein or conformational or posttranslational modification thereof, lipid, carbohydrate, organic or inorganic molecule, nucleic acid, e.g. mRNA, DNA, etc. or a portion derived from such a cell component or combinations thereof. While most parameters will provide a quantitative readout, in some instances a semi-quantitative or qualitative result will be acceptable. Readouts may include a single determined value, or may include mean, median value or the variance, etc. Variability is expected and a range of values for each of the set of test parameters will be obtained using standard statistical methods with a common statistical method used to provide single values.

Parameters of interest include detection of cytoplasmic, cell surface or secreted biomolecules, such as biopolymers, e.g. polypeptides, polysaccharides, polynucleotides, lipids, etc. Cell surface molecules, secreted molecules, and exosomes are parameters of interest, as these mediate cell communication and cell effector responses and can be more readily assayed. For example, a parameter of interest is expression of cell surface biomolecules. Epitopes present in cell surface biomolecules can be identified using specific monoclonal antibodies or receptor probes.

Test agents of interest are biologically active agents that encompass numerous chemical classes, primarily organic molecules, which may include organometallic molecules, inorganic molecules, nucleic acids, polypeptides, etc. Test agents comprise functional groups necessary for structural interaction with proteins, particularly hydrogen bonding, and may include at least an amine, carbonyl, hydroxyl or carboxyl group, frequently at least two of the functional chemical groups. The test agents can comprise cyclical carbon or heterocyclic structures and/or aromatic or polyaromatic structures substituted with one or more of the above functional groups. Test agents are also found among biomolecules, including peptides, polynucleotides, saccharides, fatty acids, steroids, purines, pyrimidines, derivatives, structural analogs or combinations thereof.

Test agents of interest include pharmacologically active drugs, genetically active molecules, etc. Compounds of interest include chemotherapeutic agents, anti-inflammatory agents, hormones or hormone antagonists, ion channel modifiers, and neuroactive agents. Exemplary of pharmaceutical agents suitable for this invention are those described in, “The Pharmacological Basis of Therapeutics,” Goodman and Gilman, McGraw-Hill, New York, N.Y., (1996), Ninth edition, under the sections: Drugs Acting at Synaptic and Neuroeffector Junctional Sites; Cardiovascular Drugs; Vitamins, Dermatology; and Toxicology, all incorporated herein by reference.

Test agents of interest include any of the classes of molecules described above, and may further comprise samples of unknown content. Of interest are complex mixtures of naturally occurring compounds derived from natural sources such as plants. While many samples will comprise compounds in solution, solid samples that can be dissolved in a suitable solvent may also be assayed. Samples of interest include environmental samples; biological samples, e.g. lysates prepared from plants, tissue samples, etc.; manufacturing samples, e.g. time course during preparation of pharmaceuticals; as well as libraries of compounds prepared for analysis; and the like. Samples of interest include compounds being assessed for potential therapeutic value, i.e. drug candidates.

The term “samples” also includes the fluids described above to which additional components have been added, for example components that affect the ionic strength, pH, total protein concentration, etc. In addition, the samples may be treated to achieve at least partial fractionation or concentration. Biological samples may be stored if care is taken to reduce degradation of the compound, e.g. under nitrogen, frozen, or a combination thereof. The volume of sample used is sufficient to allow for measurable detection, e.g., from about 0.001 ml to about 0.01 ml, from about 0.01 ml to about 0.1 ml, or from 0.1 ml to 1 ml, of a biological sample is sufficient.

Test agents can be obtained from a wide variety of sources, including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

In some cases, a test agent is a genetic agent. As used herein, the term “genetic agent” refers to polynucleotides and analogs thereof, which agents are tested in the screening assays of the present disclosure by introducing the genetic agent into a chimeric organoid (e.g., chimeric cortical organoid) of the present disclosure. The introduction of the genetic agent can results in genetic modification of a cell in the chimeric organoid (e.g., chimeric cortical organoid), or can result in modified transcription of a gene in a cell in the chimeric organoid (e.g., chimeric cortical organoid). Genetic agents such as DNA can result in genetic modification of the genome of a cell, e.g., through the integration of the sequence into a chromosome, for example using CRISPR mediated genomic engineering (see for example Shmakov et al. (2017) Nature Reviews Microbiology 15:169). Genetic changes can also be transient, where the exogenous sequence is not integrated but is maintained as an episomal agents. Genetic agents, such as antisense oligonucleotides, can also affect the expression of proteins without changing the cell's genotype, by interfering with the transcription or translation of mRNA. The effect of a genetic agent is to increase or decrease expression of one or more gene products in the cell.

Introduction of an expression vector encoding a polypeptide can be used to express the encoded product in cells lacking the sequence, or to over-express the product. Various promoters can be used that are constitutive or subject to external regulation, where in the latter situation, one can turn on or off the transcription of a gene. These coding sequences may include full-length cDNA or genomic clones, fragments derived therefrom, or chimeras that combine a naturally occurring sequence with functional or structural domains of other coding sequences. Alternatively, the introduced sequence may encode an anti-sense sequence; be an anti-sense oligonucleotide; RNAi, encode a dominant negative mutation, or dominant or constitutively active mutations of native sequences; altered regulatory sequences, etc.

Antisense and RNAi oligonucleotides can be chemically synthesized by methods known in the art. Preferred oligonucleotides are chemically modified from the native phosphodiester structure, in order to increase their intracellular stability and binding affinity. A number of such modifications have been described in the literature, which alter the chemistry of the backbone, sugars or heterocyclic bases. Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5-S-phosphorothioate, 3′-S-5-O-phosphorothioate, 3′-CH2-5′-O-phosphonate and 3′-NH-5-O-phosphoroamidate. Peptide nucleic acids replace the entire ribose phosphodiester backbone with a peptide linkage. Sugar modifications are also used to enhance stability and affinity, e.g. morpholino oligonucleotide analogs.

Test agents are screened for biological activity (modifying one or more features of a PV⁺ interneuron) by contacting a chimeric organoid (e.g., chimeric cortical organoid) of the present disclosure with the test agent. In some cases, the chimeric organoid (e.g., chimeric cortical organoid) is also subjected to one or more environmental conditions, e.g. stimulation with an agonist, electric stimulation, mechanical stimulation, etc. A change in parameter readout in response to the test agent is measured, desirably normalized, and the resulting screening results may then be evaluated by comparison to reference screening results, e.g. with PV⁺ interneurons that do not include a particular genetic modification., and the like. The reference screening results may include readouts in the presence and absence of different environmental changes, screening results obtained with other agents, which may or may not include known drugs, etc.

A test agent is generally added in solution, or readily soluble form, to the medium of cells in culture. A test agent may be added in a flow-through system, as a stream, intermittent or continuous, or alternatively, adding a bolus of the agent, singly or incrementally, to an otherwise static solution.

A plurality of assays may be run in parallel with different test agent concentrations to obtain a differential response to the various concentrations. As known in the art, determining the effective concentration of a test agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the parameter (e.g., physiology, morphology, gene expression, etc.).

Various methods can be utilized for quantifying the presence of selected parameters, in addition to the functional parameters described above. For measuring the amount of a molecule that is present, a convenient method is to label a molecule with a detectable moiety, which may be fluorescent, luminescent, radioactive, enzymatically active, etc., particularly a molecule specific for binding to the parameter with high affinity fluorescent moieties are readily available for labeling virtually any biomolecule, structure, or cell type. Immunofluorescent moieties can be directed to bind not only to specific proteins but also specific conformations, cleavage products, or site modifications like phosphorylation.

Depending upon the label chosen, parameters may be measured using other than fluorescent labels, using such immunoassay techniques as radioimmunoassay (RIA) or enzyme linked immunosorbance assay (ELISA), homogeneous enzyme immunoassays, and related non-enzymatic techniques. These techniques utilize specific antibodies as reporter molecules, which are particularly useful due to their high degree of specificity for attaching to a single molecular target. U.S. Pat. No. 4,568,649 describes ligand detection systems, which employ scintillation counting. These techniques are particularly useful for protein or modified protein parameters or epitopes, or carbohydrate determinants. Cell readouts for proteins and other cell determinants can be obtained using fluorescent or otherwise tagged reporter molecules. Cell based ELISA or related non-enzymatic or fluorescence-based methods enable measurement of cell surface parameters and secreted parameters. Capture ELISA and related non-enzymatic methods usually employ two specific antibodies or reporter molecules and are useful for measuring parameters in solution. Flow cytometry methods are useful for measuring cell surface and intracellular parameters, as well as shape change and granularity and for analyses of beads used as antibody-or probe-linked reagents. Readouts from such assays may be the mean fluorescence associated with individual fluorescent antibody-detected cell surface molecules or cytokines, or the average fluorescence intensity, the median fluorescence intensity, the variance in fluorescence intensity, or some relationship among these.

Neuronal activity parameters can be measured using any of various methods known in the art. Of particular interest for a screening assay of the present disclosure are parameters related to the electrical properties of the cells and therefore directly informative about neuronal function and activity. Methods to measure neuronal activity may sense the occurrence of action potentials (spikes). The characteristics of the occurrence of a single spike or multiple spikes either in timely clustered groups (bursts) or distributed over longer time (spike train) of a single neuron or a group of neurons indicate neuronal activation patterns and thus reflect functional neuronal properties, which can be described my multiple parameters. Such parameters can be used to quantify and describe changes in neuronal activity in a chimeric organoid (e.g., chimeric cortical organoid) of the present disclosure.

Neuronal activity parameters include, without limitation, total number of spikes (per recording period); mean firing rate (of spikes); inter-spike interval (distance between sequential spikes); total number of bursts (per recording period); burst frequency; number of spikes per burst; burst duration (in milliseconds); inter-burst interval (distance between sequential bursts); burst percentage (the portion of spikes occurring within a burst); total number of network bursts (spontaneous synchronized network activity); network burst frequency; number of spikes per network burst; network burst duration; inter-network-burst interval; inter-spike interval within network bursts; network burst percentage (the portion of bursts occurring within a network burst); salutatory migration, etc.

Quantitative readouts of neuronal activity parameters may include baseline measurements in the absence of agents or a pre-defined control condition and test measurements in the presence of a single or multiple agents or a test condition. Furthermore, quantitative readouts of neuronal activity parameters may include long-term recordings and may therefore be used as a function of time (change of parameter value). Readouts may be acquired either spontaneously or in response to or presence of stimulation or perturbation of the complete neuronal network or selected components of the network. The quantitative readouts of neuronal activity parameters may further include a single determined value, the mean or median values of parallel, subsequent or replicate measurements, the variance of the measurements, various normalizations, the cross-correlation between parallel measurements, etc. and every statistic used to a calculate a meaningful and informative factor.

Comprehensive measurements of neuronal activity using electrical or optical recordings of the parameters described herein may include spontaneous activity and activity in response to targeted electrical or optical stimulation of all neuronal cells or a subpopulation of neuronal cells within the integrated forebrain. Furthermore, spontaneous or induced neuronal activity can be measured under conditions of selective perturbation or excitation of specific PV⁺ interneurons, as discussed above.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A method for generating an enriched population of parvalbumin-positive interneurons, the method comprising culturing a population of mouse primary neuronal progenitors in a human brain organoid or a primary brain slice of human origin, wherein the mouse primary neuronal progenitors differentiate into parvalbumin-positive interneurons in the brain organoid or the primary brain slice, thereby generating an enriched population of parvalbumin-positive interneurons.

Aspect 2. The method of aspect 1, further comprising determining the number of parvalbumin-positive interneurons in the brain organoid or the primary brain slice.

Aspect 3. The method of aspect 1, wherein the enriched population of parvalbumin-positive interneurons is generated within 2 days of said culturing.

Aspect 4. The method of any one of aspects 1-3, wherein at least 50% of the population of primary neuronal progenitors differentiate into parvalbumin-positive interneurons.

Aspect 5. The method of any one of aspects 1-4, wherein the mouse primary neuronal progenitors are medial ganglionic eminence (MGE) neuronal progenitors or post-mitotic somatostatin-positive interneurons.

Aspect 6. The method of aspect 5, wherein the MGE neuronal progenitors are genetically modified to reduce expression of or to render a target gene non-functional.

Aspect 7. The method of aspect 6, wherein the target gene is selected from PVALB, SST, MEF2C, GAD2, DLX5, DLX6, NKX2-1, MTOR, TSC1, TSC2, MECP2, PTEN, RYK, CHD8, ERBB4, MAKA, SCN1A, EGFR, SYT2, GLI2, and LHX6.

Aspect 8. The method of aspect 5, wherein the MGE neuronal progenitors are genetically modified with a heterologous nucleic acid comprising a nucleotide sequence encoding a heterologous gene product of interest.

Aspect 9. The heterologous gene product is a polypeptide selected from PVALB, SST, MEF2C, GAD2, DLX5, DLX6, NKX2-1, MTOR, TSC1, TSC2, MECP2, PTEN, RYK, CHD8, ERBB4, MAKA, SCN1A, EGFR. SYT2, GLI2, and LHX6, and where the polypeptide comprises one or more mutations compared to wild-type.

Aspect 10. The method of any one of aspects 1-9, wherein the enriched population comprises from about 10³ to about 10⁷ parvalbumin-positive interneurons.

Aspect 11. The method of aspect 10, wherein at least 80% of the enriched population are parvalbumin-positive interneurons.

Aspect 12. The method of any one of aspects 1-11, further comprising:
a) contacting the population of parvalbumin-positive interneurons with a test agent; and
b) determining the effect of the test agent on a feature of the parvalbumin-positive interneurons.

Aspect 13. The method of aspect 12, wherein the feature is viability, physiology, morphology, connectivity, or gene expression.

Aspect 14. The method of aspect 12, wherein the feature is expression of a gene product, wherein the gene product is an mRNA or a polypeptide encoded by a gene selected from PVALB, SST, MEF2C, GAD2, DLX5, DLX6, NKX2-1, MTOR, TSC1, TSC2, MECP2, PTEN, RYK, CHD8, ERBB4, MAKA, SCN1A, EGFR, SYT2, GLI2, and LHX6.

Aspect 15. A chimeric organoid comprising: a) human brain organoid; and b) an enriched population of mouse parvalbumin-positive interneurons.

Aspect 16. The chimeric organoid of aspect 15, wherein the enriched population comprises from about 10³ to about 10⁷ parvalbumin-positive interneurons.

Aspect 17. The chimeric organoid of aspect 18, wherein at least 80% of the enriched population are parvalbumin-positive interneurons.

Aspect 18. The chimeric organoid of any one of aspects 15-17, wherein the mouse PV⁺ interneurons are within a perineuronal net.

Aspect 19. The chimeric organoid of any one of aspects 15-17, wherein the chimeric organoid is a chimeric cortical organoid.

Aspect 20. A method of identifying an agent that enhances function of a parvalbumin-positive interneuron, the method comprising:
a) contacting the chimeric organoid of any one of aspects 15-19 with a test agent; and
b) determining the effect of the test agent on a feature of the parvalbumin-positive interneurons, wherein a test agent that enhances the feature is a candidate agent for enhancing the function of the parvalbumin-positive interneuron.

Aspect 21. The method of aspect 20, wherein the feature is viability, physiology, morphology, connectivity, or gene expression.

Aspect 22. The method of aspect 20, wherein the feature is interaction with a perineuronal net.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### Host Environment Biases MGE-interneuron Fate

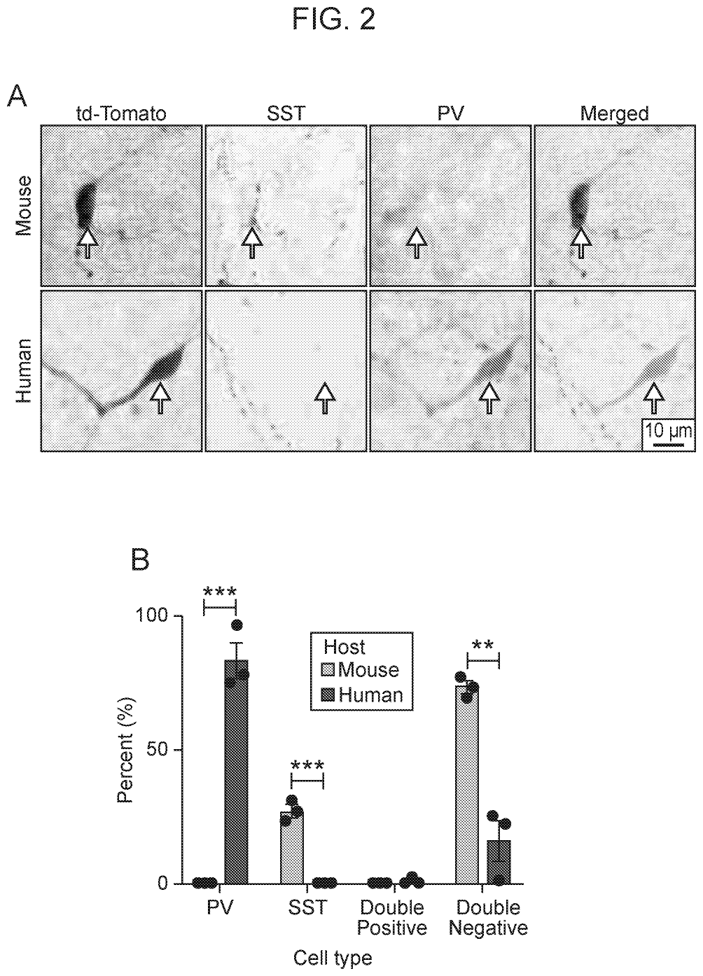

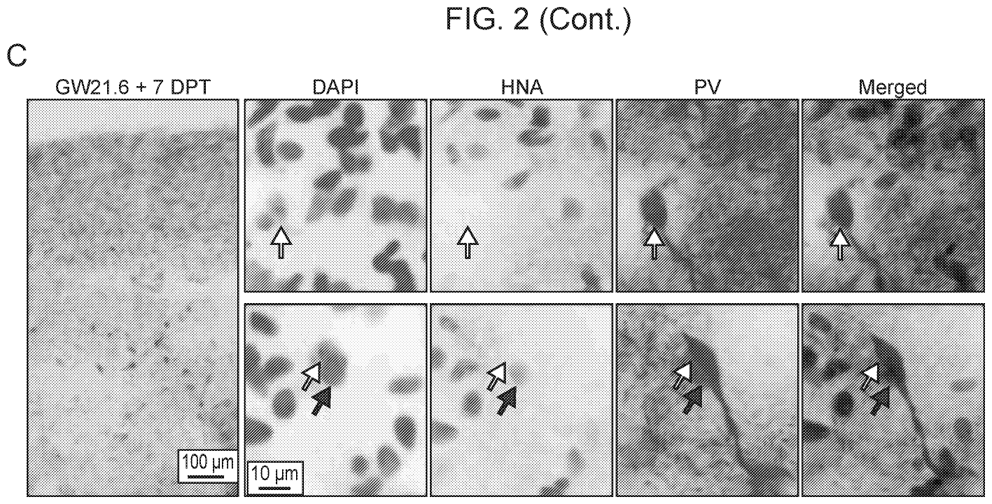

To understand whether the host environment can bias the identity of neuronal progenitors, E13.5 MGE progenitors were transplanted into organotypic cultures of human and mouse embryonic hosts (FIG. 2A). The previously described Nkx2.1-Cre mouse line was used, which specifically labels MGE-neuronal progenitors and its descendants (Xu et al., 2008) and crossed it to the Ai14 mouse line, which contains a floxed td-Tomato reporter in the ROSA26 locus (Madisen et al., 2010). The identity of the differentiated mouse INs were assessed by immunohistochemistry. Strikingly, xenotransplanted MGE progenitors differentiated into dramatically distinct fates depending on the host species. Specifically, transplantation of mouse MGE progenitors into E14.5 wild-type (WT) mouse brain organotypic slices yielded 27.00+3.94% of SST interneurons 7 days post-transplant (DPT) while 72.99+3.94% of descendants were immunonegative for both SST and PV (FIG. 2B). Notably, across 3 transplantation batches, no cell was immunopositive for PV (FIG. 2B), consistent with the fact that in mouse, PV is not upregulated until the third postnatal week. On the other hand, transplantation of mouse MGE progenitors into human gestational week (GW) 22 primary cortical slices resulted in 82.94+11.60% PV positive interneurons, and 16.81+13.29% double negative interneurons (n=3) 7 DPT (FIG. 2B). Importantly, while human MGE-derived interneurons have already migrated into the cortex well before GW22, PV is not upregulated during the second embryonic trimester and consistently evidence of PV immunoreactivity in human cells was not found. On the other hand, SST positive interneurons in the host are readily observed (FIG. 2B).

An additional experiment was performed in which WT unlabeled interneuron progenitors were transplanted into a GW21.6 cortex of a fourth individual (FIG. 2C). Mouse and human cells were distinguished a posteriori by immunostaining against the human nuclear antigen (HNA). Several PV positive interneurons were detected at 7 DPT, all of which were HNA negative. Because PV is upregulated 7 DPT (equivalent to mouse postnatal day 0), this phenomenon indicates that the human cortical environment not only induces PV fate in mouse cells, but does so at an accelerated rate, 3 weeks before expected in vivo.

### Development of Chimeric Cortical Organoid Models for Longitudinal Interrogation of Circuit Assembly

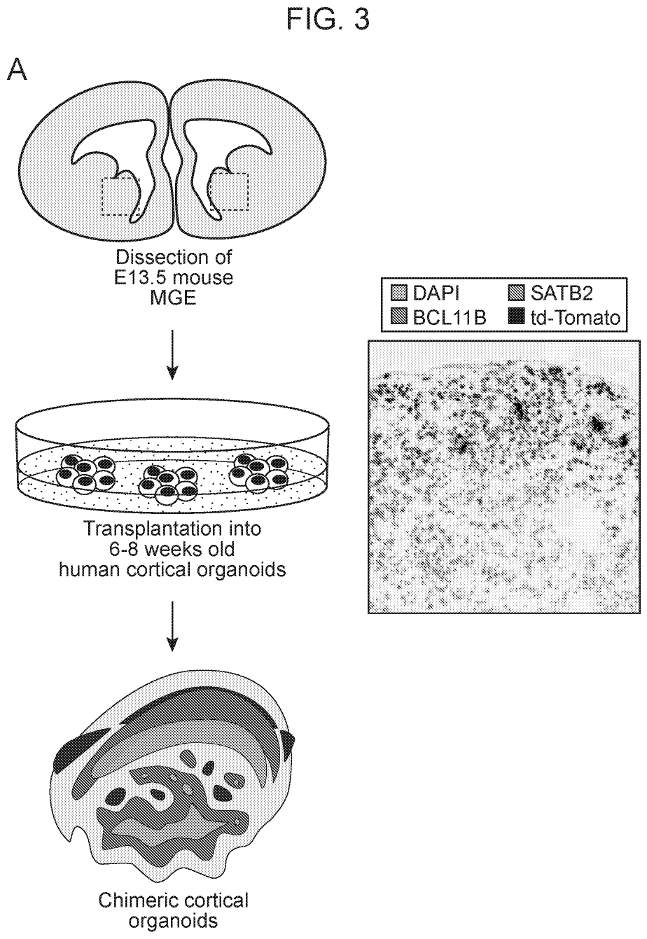

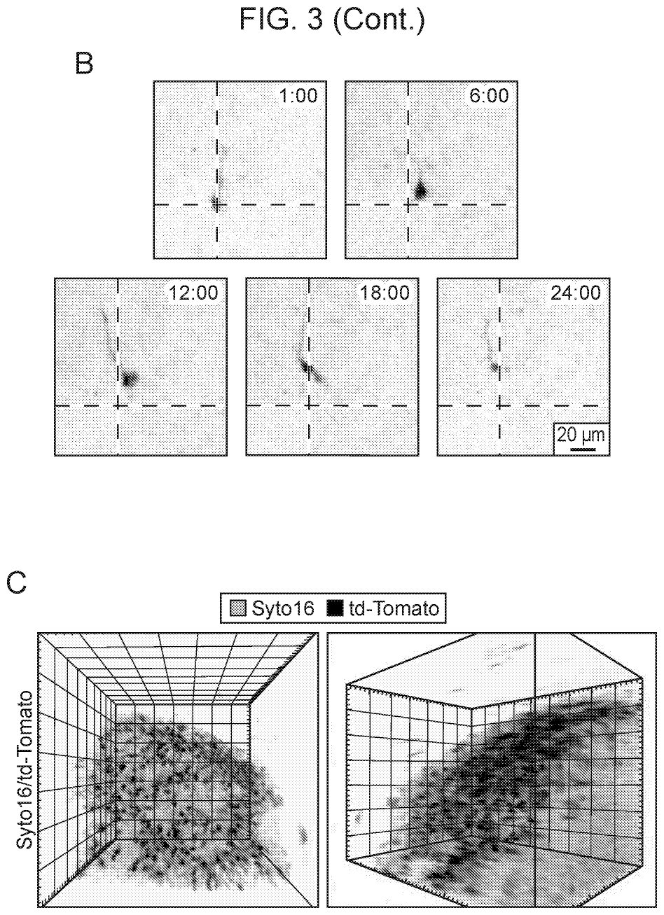

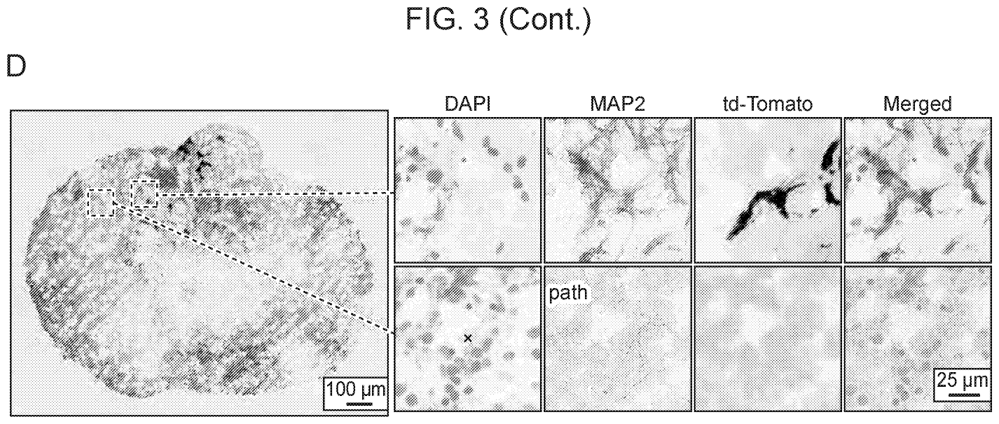

Human organotypic cortical cultures suffer from many limitations, including the long-term viability of neurons, as well as the scarcity of donors (Humpel, 2015). Induced pluripotent stem cell (iPSC)-derived organoids, on the other hand, have emerged as long-term 3D models of cortical development, tissue architecture and function (Mostajo-Radji et al., 2020; Pollen et al., 2019; Quadrato et al., 2017). A long-term chimeric model was developed by transplanting E13.5 MGE progenitors into human cortical organoids (FIG. 3A). Deep layer PNs are needed for the migration of MGE-derived INs into the cerebral cortex during embryonic development (Lodato et al., 2011). 6-8 weeks old human cortical organoids were used as hosts (FIG. 3A), as this time point corresponds to the peak of deep-layer PN neurogenesis (Pollen et al., 2019).

To assess whether mouse INs are migratory within human hosts, longitudinal live imaging of chimeric cortical organoids was performed 1 DPT hourly for 24 hours. Constant exploratory behavior was observed, neurite branching and nucleokinesis (FIG. 3B, suggesting that INs actively adapt to their new host environment.

The final position of INs was then evaluated by performing light-sheet microscopy of whole organoids 5 weeks post-transplant (WPT), well after IN maturation. In order to image throughout the entire organoid, tissue clearing was performed prior to imaging. Mouse INs were observed throughout the entirety of the organoid (FIG. 3C). However, the vast majority of INs were localized at the periphery of the organoids (FIG. 3C). In general, newborn PNs localize at the periphery of organoids, while the center contains a necrotic core (Giandomenico et al., 2021). This phenomenon leaves two possibilities to explain the location of the grafted INs: 1) INs are attracted to the peripheral PNs in the organoids, as observed in vivo (Lodato et al., 2011) or 2) Unlike other cell types (Bhaduri et al., 2020; Pham et al., 2018; Schmunk et al., 2020), INs could be incapable of penetrating to the core of the organoid. In order to distinguish between these possibilities occasionally organoids are generated containing few postmitotic neurons, many of which localize within the organoid. Mouse INs were transplanted into these organoids and performed immunostaining against the pan-neuronal marker MAP2. Strikingly, INs were always found near the MAP2-positive clusters, regardless of their location (FIG. 3D). Importantly, MAP2-negative areas were also deficient in transplanted INs, even if these areas were in the periphery of the organoid (FIG. 3D). Together, it was concluded that transplanted mouse INs are attracted to human PNs.

FIG. 3A-3D. A. The MGE of mouse E13.5 embryos was microdissected and transplanted into 6-8 weeks old human organoids. B. Interneuron migration 1 DPT for 24 hours. Representative longitudinal live imaging of a single migratory IN. The position of the cell soma 1 hour after the start of the experiment is marked for reference. C. Lightsheet imaging of a transplanted organoid. Syto16 labels all nuclei. D. INs migrate to neuronal (MAP2 positive) regions of the organoid. On left: Whole section of an organoid. On right, example of MAP2 rich and MAP2 poor regions of the organoid.

Previous work performing monosynaptic tracing of human stem cell-derived PNs into mouse hosts has shown that mouse INs can effectively synapse into human cortical PNs (Real et al. 2018), suggesting that in the transplantation paradigm mouse INs could integrate into human organoids. The integration of transplanted INs into human organoid hosts was then assessed using a variety of genetic, molecular and physiological approaches. First, the Ai34 reporter line was taken advantage of, in which a floxed Synaptophysin-tdTomato fusion gene is inserted in the ROSA26 locus (Daigle et al., 2018).

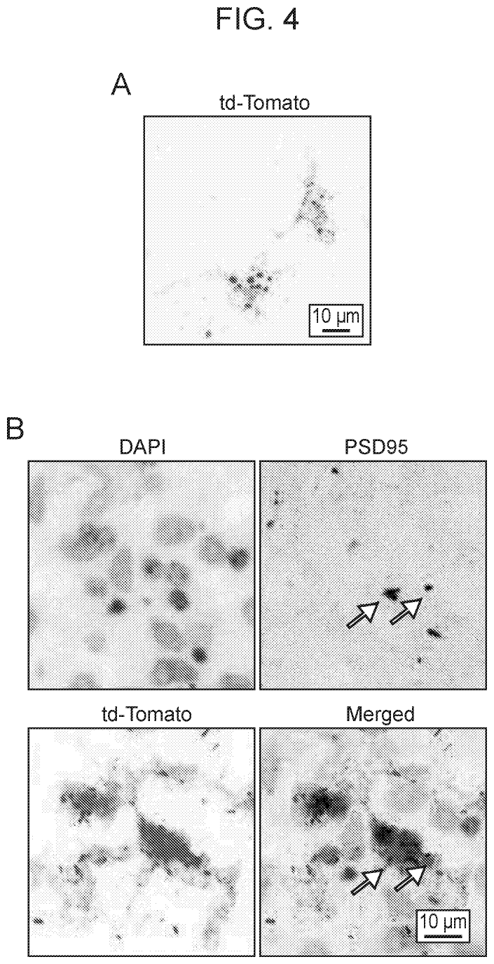

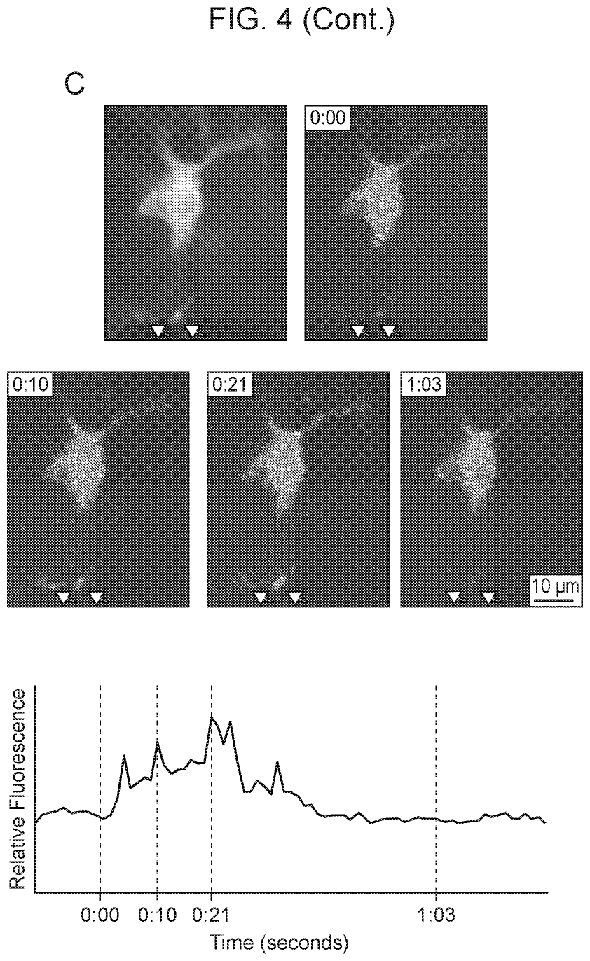

Finally, in order to interrogate the long-term integration of transplanted INs, calcium imaging was performed. The Ai96 mouse line was used, which contains the genetically-encoded calcium indicator GCaMP6s floxed in the ROSA26 locus (Madisen et al., 2015). These mice were crossed to the Nkx2.1-Cre mouse and allowed the INs to develop for 4 months post-transplantation (MPT). Performing calcium imaging in MGE-derived INs, and particularly PV-positive INs is not trivial, as PV is a slow calcium buffer which reduces the peak of calcium transients (Caillard et al., 2000), therefore affecting the imaging of such transients (FIG. 4C). The analysis was concentrated in axodendritic processes, which are effective readouts of synaptic integration (Ali and Kwan, 2019). At baseline, and without any external stimuli, strong calcium transients were observed in axodendritic processes (FIG. 4C). Altogether, it was concluded that transplanted mouse INs integrate and wire with human PNs in chimeric cortical organoid models.

FIG. 4A-4C. A. The NKX2.1: Ai34 mouse line showed strong SYP presence in the transplanted INs 5 WPT, indicating presynaptic vesicles. B. The postsynaptic excitatory marker PSD95 was observed in transplanted INs 5 WPT, suggestive of afferent excitatory synapses. C. Calcium imaging using the NKX2.1: Ai96 mouse line 4 MPT showed axodendritic calcium transients.

### Human Cortical Organoids Instruct PV-positive Interneuron Fate

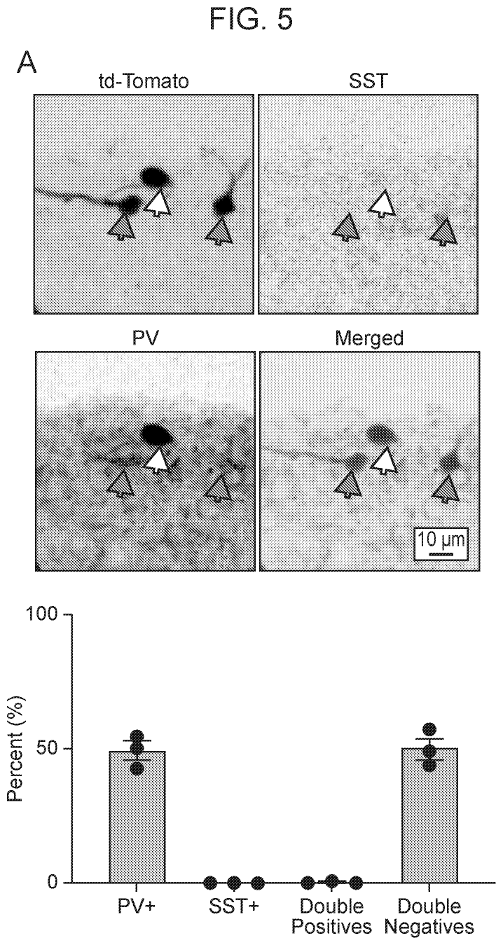

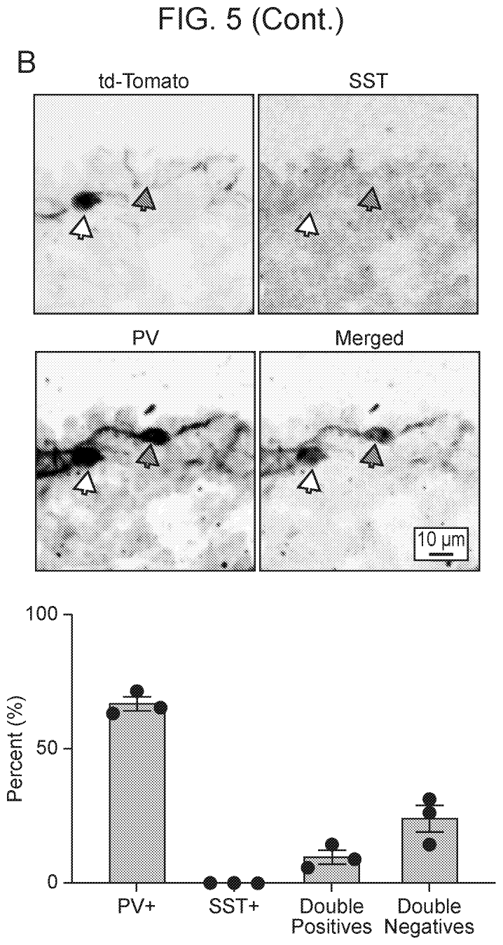

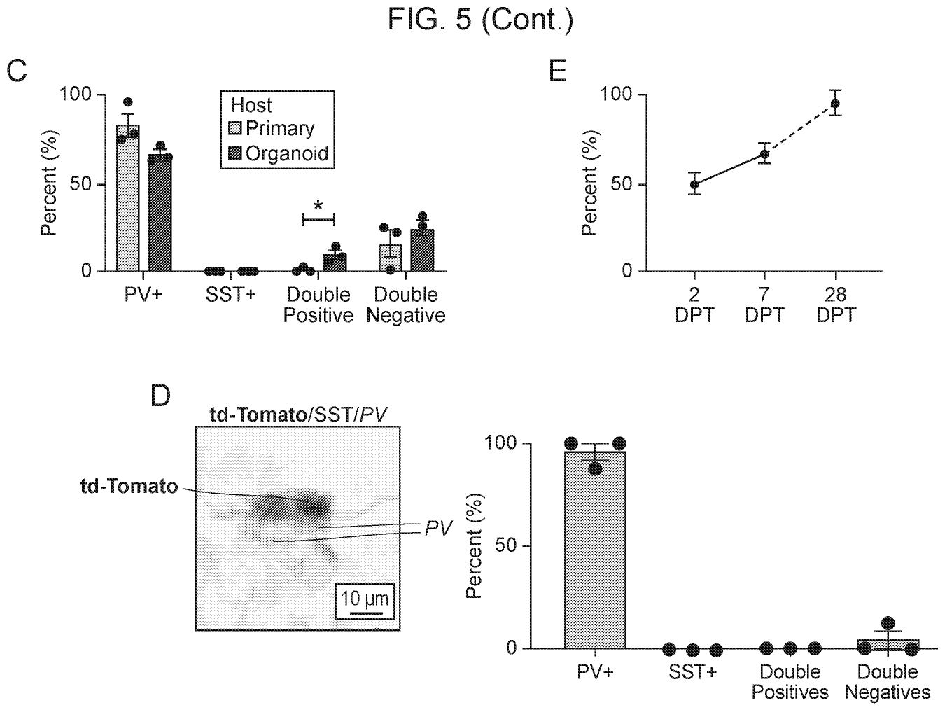

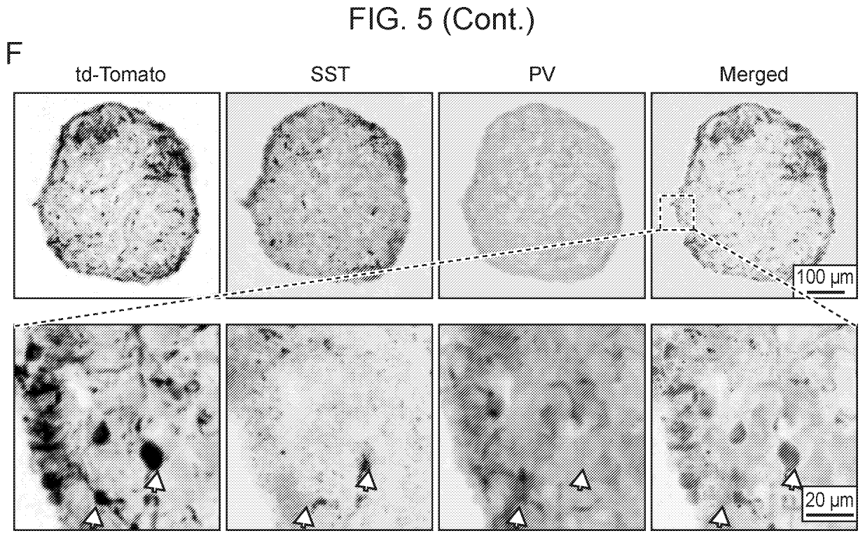

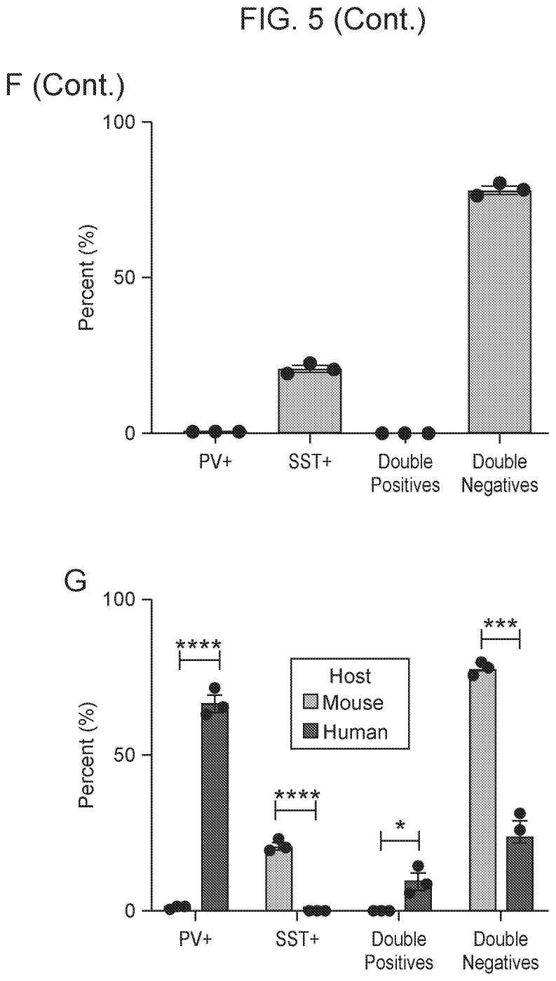

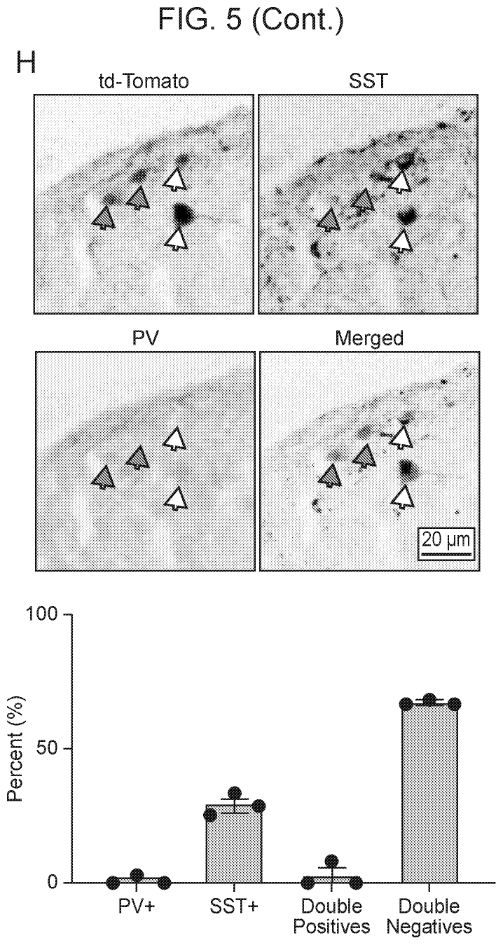

Considering that mouse INs effectively integrate into human organoids, determining if human organoids could recapitulate the PV fate bias observed in organotypic cultures was focused on next. The identity of INs 2 DPT was first analyzed, a timepoint in which cells were still migratory (as shown in FIG. 3B). Strikingly, ˜50% of transplanted INs were positive for PV at this time (FIG. 5A) (49.51±6.11% PV positive, 0% SST positive, 0.40±0.70% PV and SST double positive and 50.07±6.67% double negative).

Then, the identity of transplanted INs were analyzed at later time points. Indeed, immunostaining against PV and SST in transplanted organoids revealed that 66.56±4.38% of transplanted INs upregulated PV 7 DPT (FIG. 5B), similarly to what was observed in transplants into organotypic cultures (FIG. 5C, p>0.05). Of note, any cell that upregulated SST alone was not observed, although 9.57÷4.29% of cells were double positive for SST and PV. By 28 DPT the percentage of PV positive INs increased to 95.83±7.22% (0% SST positive, 0% double positive, 4.16±7.21% double negative) (FIG. 5D). Altogether, a progressive acquisition of PV fate was observed, at an accelerated and higher percentage than expected in nature, suggesting that extrinsic cues in the human 3D context can instruct the fate of the MGE progenitors.

Given the striking results observed in the xenotransplantations, an analysis was conducted to determine whether or not transplantation into mouse organoids would yield similar results. Mouse organoids were generated by dissociating E14.5 mouse cortices and reaggregating them in neuronal differentiation media. This approach, herein refer to as “aggregoids” yields cortical neurons of upper and deep layer identity in a 3D context. Similar to transplantation into organotypic cultures, 1.01±0.43% of transplanted INs are PV-positive 7 DPT. On the other hand, 20.91±1.61% of transplanted INs were SST positive and 78.07±1.93% did not upregulate any of the two markers (FIG. 5E). The transplanted aggregoids 28 DPT were then analyzed, a time point equivalent to the peak of PV upregulation in the in vivo context. It would be expected that if IN PV fate was genetically instructed, the majority of transplanted INs would be PV-positive at this time point. Yet, only 0.95±1.65% of transplanted INs were positive for PV (p>0.05 compared to 7 DPT), while 28.97±4.18% cells were SST positive and 67.30±1.09% lacked both markers. Together, these results suggest that the environment shapes INs fate.

FIG. 5A-5H. (A-E) Transplantation of mouse INs into human organoids. A. Representative image and quantification of the fate of transplanted INs into human organoids 2 DPT. White arrow=a mouse IN immunopositive for PV. Yellow arrows=mouse INs immunonegative for PV. n=3 organoids from 2 differentiations. B. Representative image and quantification of the fate of transplanted INs into human organoids 7 DPT. White arrow=a mouse IN immunopositive for PV. Magenta arrow=mouse IN that failed to recombine the td-Tomato. n=3 organoids from 3differentiations. C. Comparison between cell types generated 7 DPT in primary (grey) or organoid (black) human hosts. The data included is the same as 2B and 5B. D. Representative image and quantification of the fate of transplanted INs into human organoids 28 DPT. n=3 organoids from 2 differentiations. E. Progressive acquisition of PV fate in human organoid hosts. F-H. Transplantation of mouse INs into mouse aggregoids. F. Representative image and quantification of the fate of transplanted INs into mouse aggregoids 7 DPT. n=3 aggregoids from 2 differentiations. G. Comparison of populations generated in mouse aggregoids (grey) or human organoids (black) hosts 7 DPT. H. Representative image and quantification of the fate of transplanted INs into mouse aggregoids 28 DPT. n=3 aggregoids from 2 differentiations. *=p<0.05; ***=p<0.001; ****=p<0.0001. Error bars represent SEM.

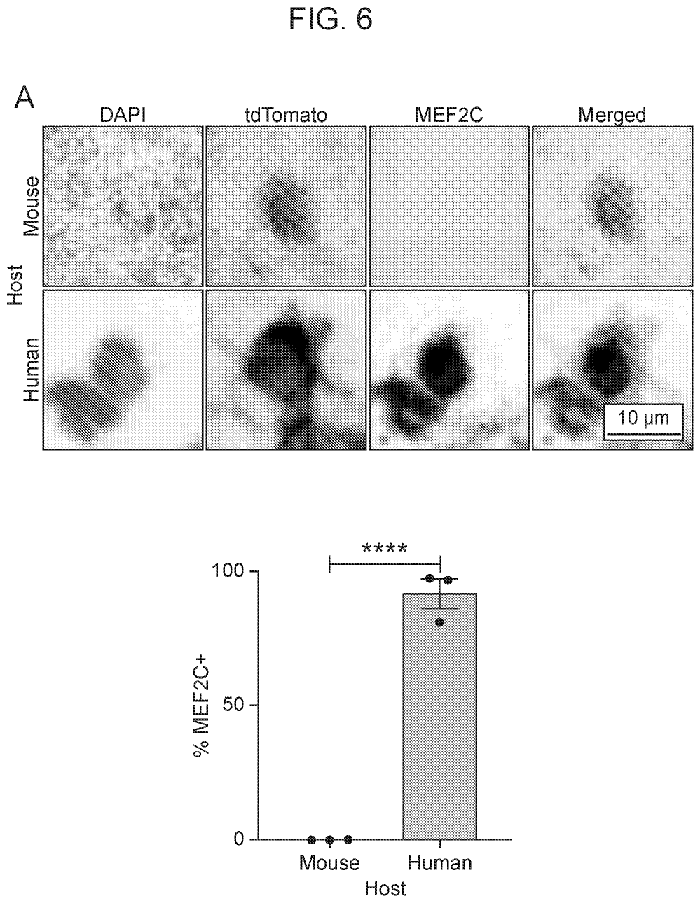

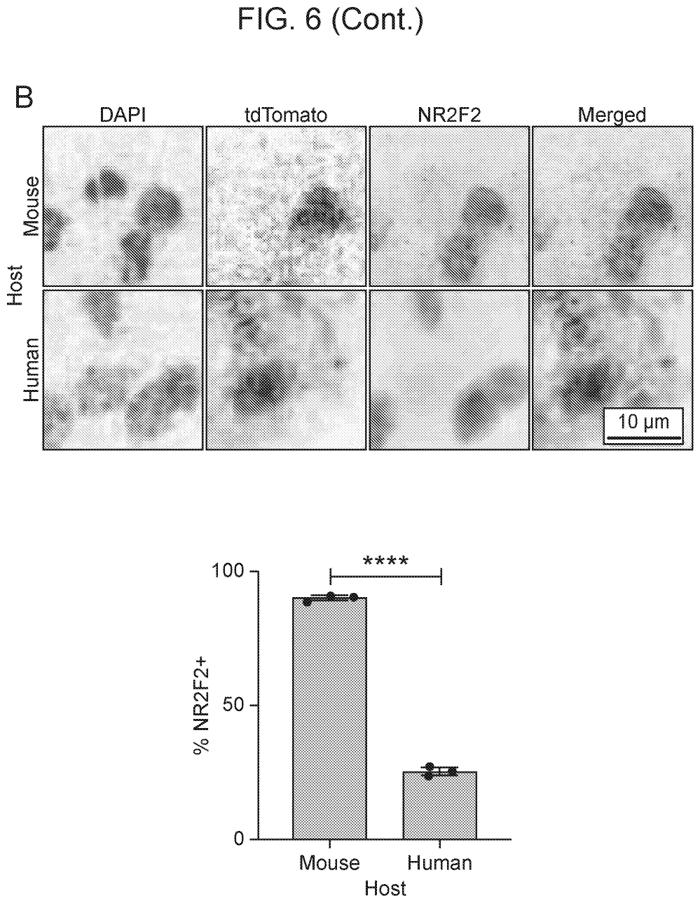

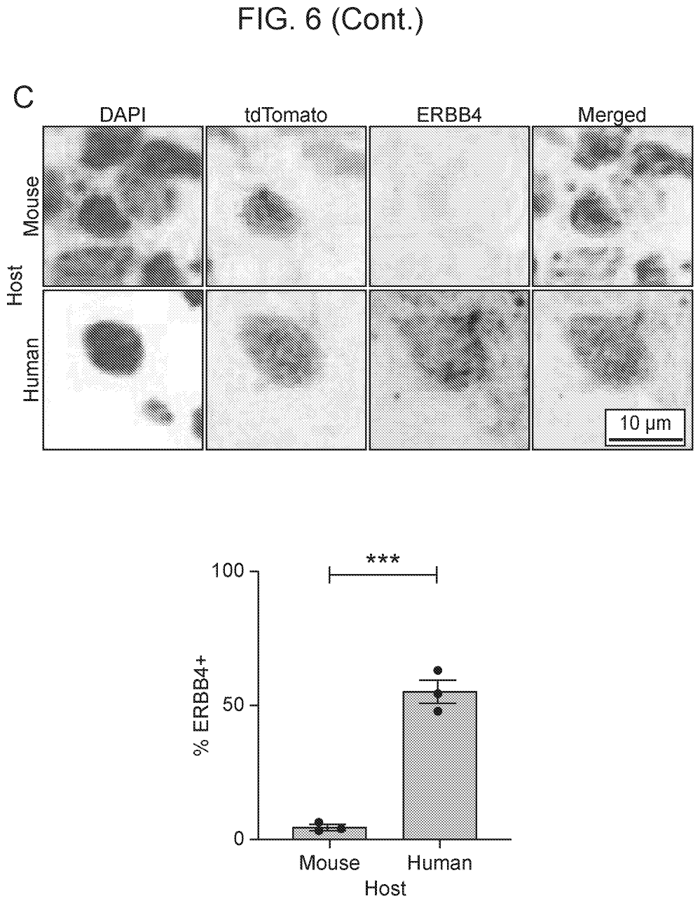

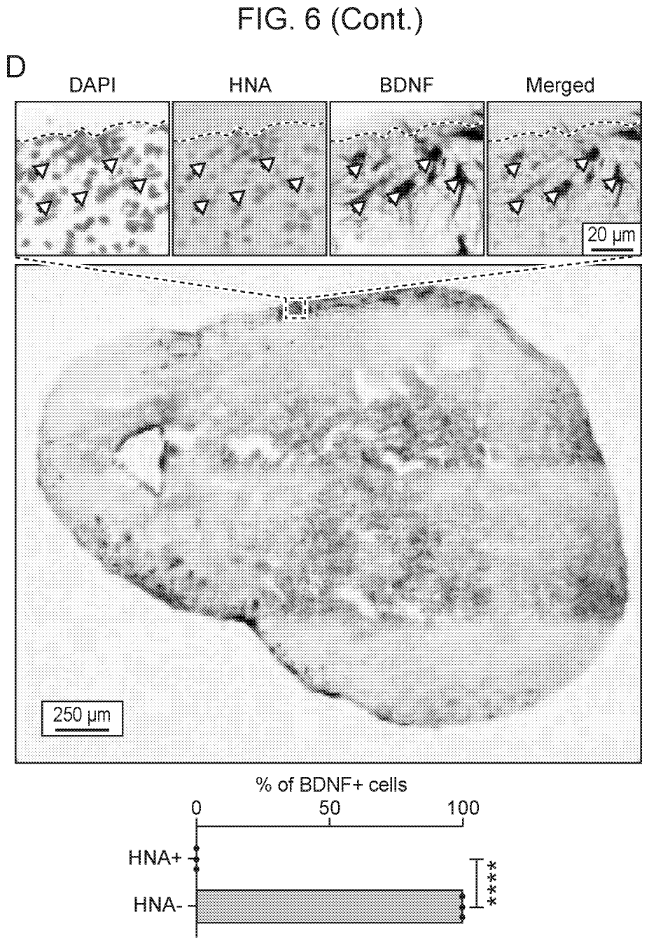

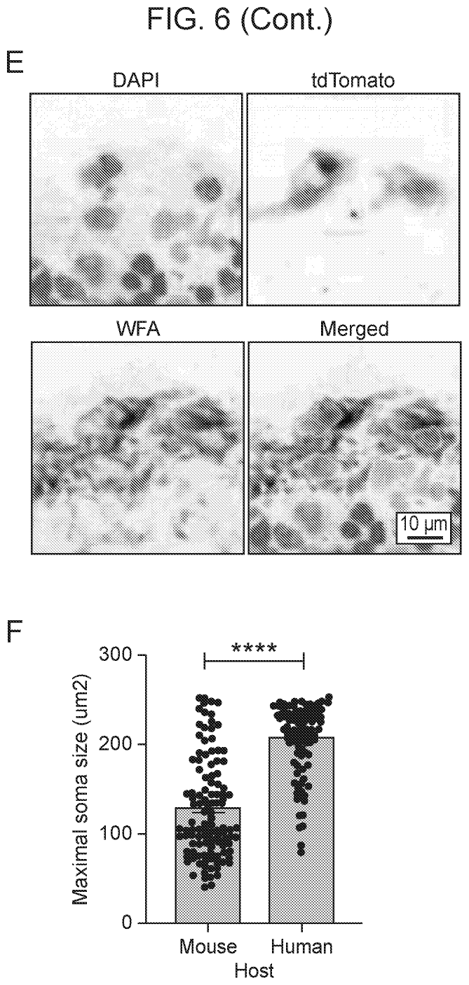

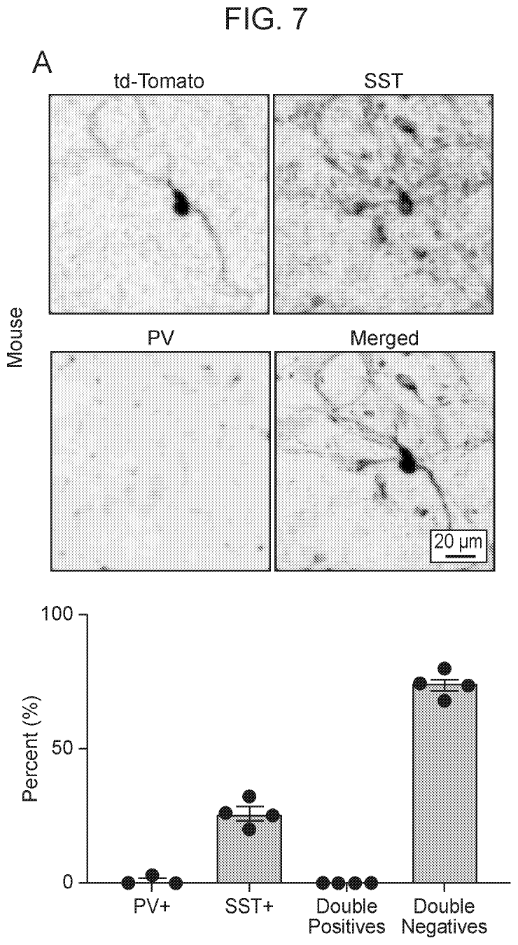

While PV and SST are terminal markers of distinct IN populations, to date few additional markers distinguishing between both fates have been identified, in part due to the relatively different developmental timelines between both cell types. The most prominent of such genes are the transcription factors MEF2C and NR2F2 (also known as COUP-TF2). MEF2C has been proposed as an early marker of PV-fated INs (Mayer et al., 2018). MEF2C has also been shown as upregulated in mature human PNs (Pollen et al., 2019). NR2F2, on other hand, has been shown to promote SST identity and repress PV fate (Hu et al., 2017). The percentages of MEF2C-positive and NR2F2 positive-INs 7 DPT were then quantified. Remarkably, it was found that 90.38±8.99% of INs transplanted into human organoids upregulated MEF2C at this time point (FIG. 6A), consistent with our findings that human 3D cultures upregulate PV. In contrast, no MEF2C positive IN was identified in transplants into mouse aggregoids (p<0.0001) (FIG. 6A). The opposite result was true for NR2F2, where 25.26%±2.19% of the INs transplanted into human organoids were positive for NR2F2, while 90.09±1.50% of INs transplanted into mouse aggregoids were positive for this marker (p<0.0001) (FIG. 6B).

In mature cortical circuits, it has previously been shown that ERBB4 is a marker of PV INs, while essentially no cortical SST-positive IN is immunopositive for ERBB4 (Sun et al., 2016). Immunostaining against this marker a 7 DPT once again showed dramatic differences depending on the host species of the INs: while the majority of INs transplanted into human organoids were positive for ERBB4 (55.45%±7.49%), this gene was rarely detected in INs transplanted into mouse aggregoids (4.95±1.76%; p<0.001) (FIG. 6C).

Previous work has shown that a subset of PV-positive INs are immunopositive for BDNF (Huang et al. 1999; Tomas et al., 2020), while no other IN subtype has been reported to synthesize BDNF. BDNF has been shown to regulate the maturation of PV INs (Huang et al., 1999). However, cortical organoids rarely express this gene in any cells (Pollen et al., 2019; Quadrato et al., 2017) and the organoid production protocols used in this study do not contain BDNF. Immunostaining for BDNF and HNA was performed, allowing the ability to distinguish the species of the BDNF expressing cells. Extensive expression of BDNF was found in the transplanted organoids, exclusively in HNA-negative cells, indicating that the source of BDNF in the transplants are mouse cells (FIG. 6D).

Then, analysis on perineuronal nets (PNNs) was focused on. PNNs are extracellular matrix (ECM) assemblies that preferentially ensheath PV-positive INs. allowing their maturation and closure of circuit plasticity (Wen et al., 2018). In the brain, PNNs are extrinsically instructed by neighboring neurons, including PNs and PV-negative INs (Su et al., 2017). One of the genes that has been involved in the regulation of PNNs is COL19A1 (Su et al., 2017), which has previously shown that it is highly expressed in mature neurons of cortical organoids (Pollen et al., 2019) and developing deep-layer PNs in the human prefrontal cortex (Nowakowski et al., 2017). PNNs were labeled using biotin-conjugated Wisteria floribunda agglutinin (WFA), a lectin that specifically binds N-acetylgalactosamines in PNNs (Su et al., 2017). Strong WFA labeling was found surrounding transplanted INs, suggesting that human organoid hosts can instruct PNNs in grafted PV INs (FIG. 6E).

FIG. 6A-6F. A. Representative images and quantification of MEF2C in mouse aggregoids and human organoids 7 DPT (n=3 of each; 2 differentiations). B. Representative images and quantification of NR2F2 in mouse aggregoids and human organoids 7 DPT (n=3 of each; 2 differentiations). C. Representative images and quantification of ERBB4 in mouse aggregoids and human organoids 7 DPT (n=3 of each; 2 differentiations). D. A representative image showing BDNF and HNA staining in transplanted human organoids 5 WPT. All BDNF positive cells were negative for HNA. n=4 organoids from 3 differentiations. E. Labeling of PNNs using biotinylated WFA 5 WPT. n=4 organoids from 3 differentiations. F. Maximal soma size of INs transplanted into mouse or human organoid hosts. n=109 INs transplanted into mouse aggregoids and n=106 INs transplanted into human organoids. ***=p<0.001; ****=p<0.0001. Error bars represent SEM.

Finally, the maximal soma area of transplanted INs was measured in mouse and human hosts. Neuronal size is commonly used as a parameter of neuronal identity (Ye et al., 2015). Moreover, PV-positive INs are among the largest INs in the cortex (Kooijmans et al., 2020; Malik et al., 2019). It was found that while INs transplanted into mouse aggregoids have a maximal cell soma area of 128.33±65.90 um2, transplants into human INs have a 1.6× increase in soma size, with a cell soma area of 207.33±39.03 um2 (FIG. 6F), consistent with previous reports for the same measurement on mouse IN populations (Malik et al., 2019). Together, with the upregulation of PV itself, as well as the upregulation of MEF2C, ERBB4 and BDNF, the downregulation of NR2F2 and SST, the assembly of PNNs in transplanted INs and the increase in cell size, our data indicates that the human 3D cortical system effectively instructs the acquisition of PV fate.

### Other Coculture Models Fail to Instruct PV Fate

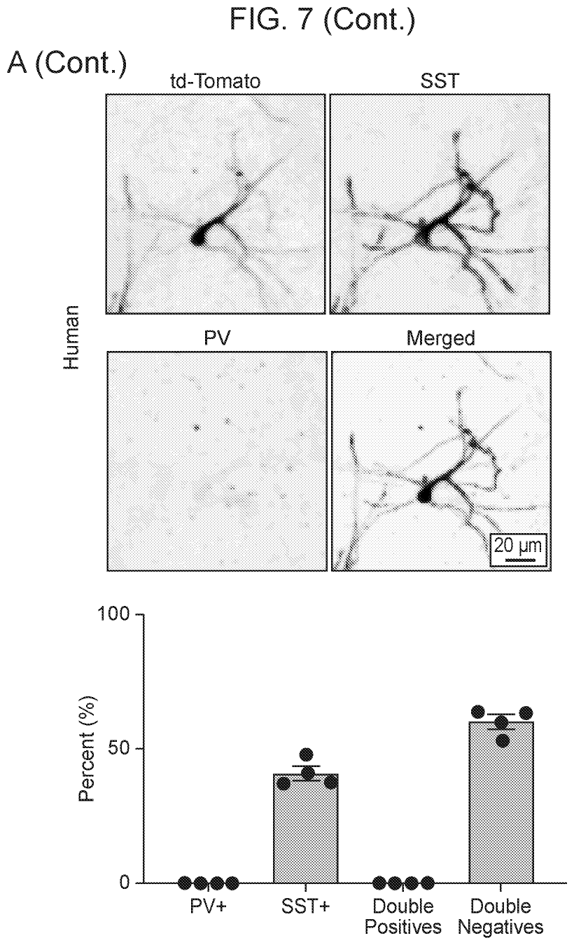

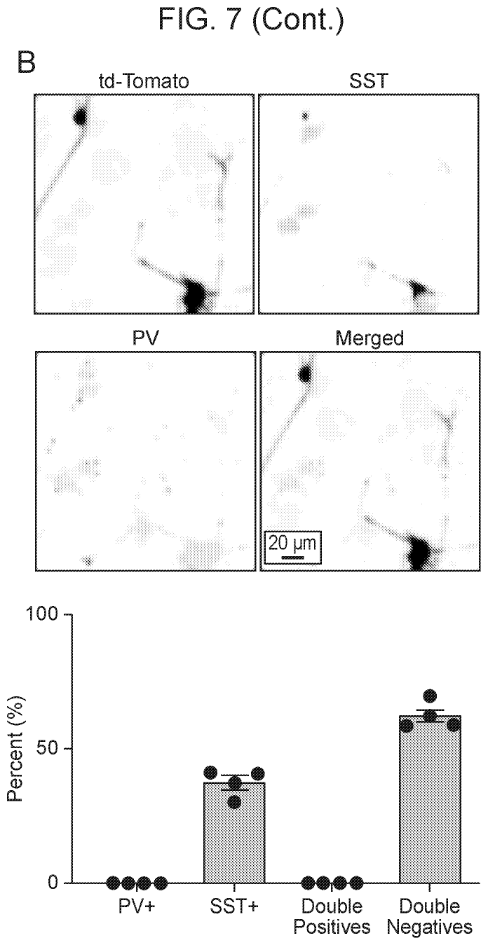

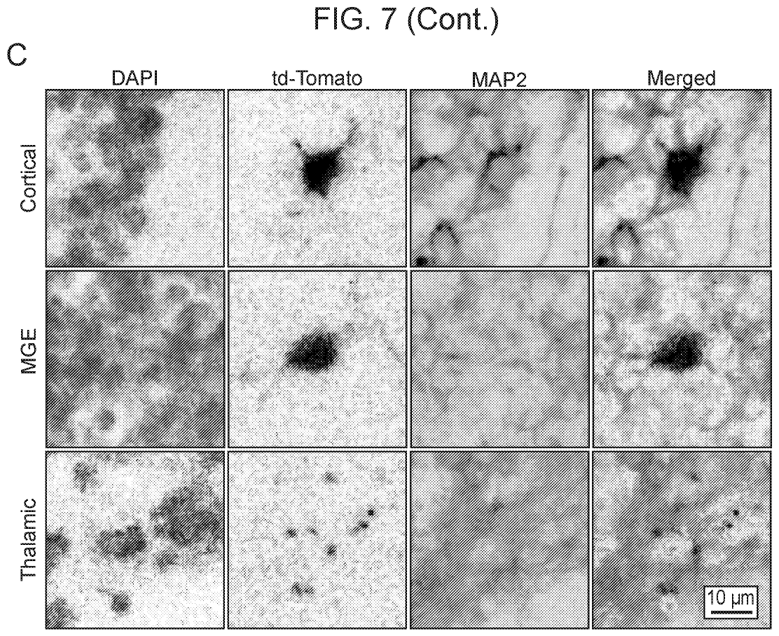

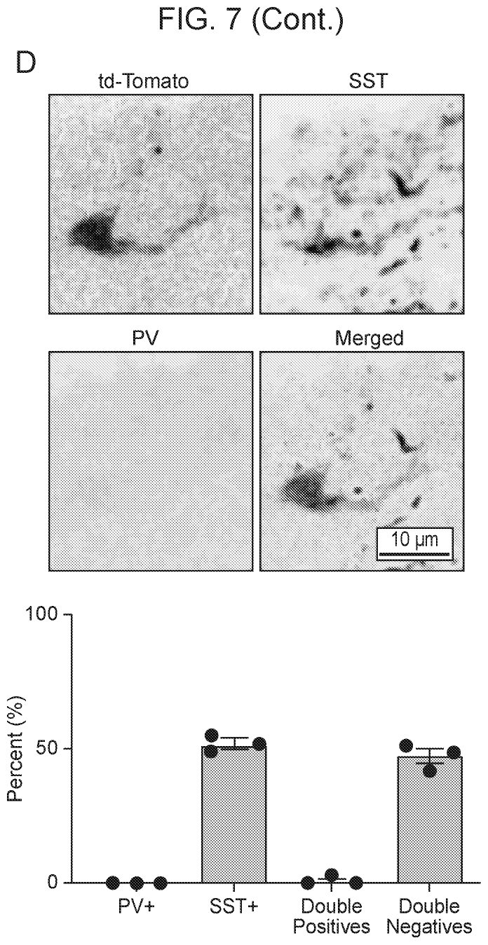

To further dissect whether the 3D cortical environment was not only sufficient to instruct PV fate in MGE progenitors, but was also necessary, MGE progenitors were cocultured with dissociated cortical cells from E14.5 mice and GW22 human hosts in 2D. After 7 days in culture (DIC) the identity of the grafts was assessed by immunostaining. No transplanted IN was positive for PV. Moreover, the results show different proportions of SST-positive INs across conditions: Whereas coculture with mouse cortical cells yields 25.85±4.93% SST INs, culturing MGE progenitors with human cortical cells yields a 1.7× fold increase in SST differentiation, reaching 40.61±4.94% SST INs (p<0.01) (FIG. 7A).

Given that coculturing INs with human cortical cells yielded a higher percentage of SST-INs, whether these differences were instructed by cell-to-cell interactions or by diffusible cues was investigated. To answer this question, mouse MGE progenitors were cultured in the presence of media conditioned by primary human cortical cells. It was found that 7 DIC the proportion of differentiated SST-positive INs was indistinguishable between INs grown in conditioned media or INs cocultured with primary human cells (37.49±5.08% SST-positive INs, p>0.05) (FIG. 7B), indicating that the extrinsic cues from the cortical cells were diffusible in the media.

Finally, whether the specification of PV-positive INs in the chimeric transplants required a cortical environment or whether the PV marker would be upregulated in other 3D human brain contexts was investigated. Mouse MGE INs were transplanted into human MGE organoids from one IPSC line and one embryonic stem (hES) cell line and human thalamic organoids from three different IPSC lines. At 7 DPT, transplantation of mouse MGE-INs into human MGE organoids yielded 51.78±3.03% SST-positive INs and no transplanted IN was PV-positive alone, although 1.07±1.87% of INs were double positive for PV and SST (FIGS. 7C and 7D). On the other the vast major INs transplanted into thalamic organoids died shortly after transplantation (FIG. 7C), in agreement with previous work that demonstrated that thalamic INs do not originate from the MGE (Jager et al., 2020). These results demonstrate that the 3D cortical environment is necessary for PV fate acquisition.

FIG. 7A-7D. A. Representative images and quantifications of INs cocultured with primary mouse and human cortical cells. No PV-positive IN is observed. n=4 replicates from 2 differentiations for each condition. B. Representative images and quantifications of INs cultured in media conditioned by primary human cortical cells. n=4 replicates from 2 differentiations for each condition. C. Representative images of INs transplanted into cortical, MGE and thalamic organoids. The pan neuronal marker MAP2 is included for reference. S. Representative image and quantification of the fate of transplanted INs into human MGE organoids 7 DPT. Of note, additional SST positive cells are observed from the host. n=3 organoids from 2 differentiations. Error bars represent SEM.

### Non Cell Autonomous Instruction of PV Fate

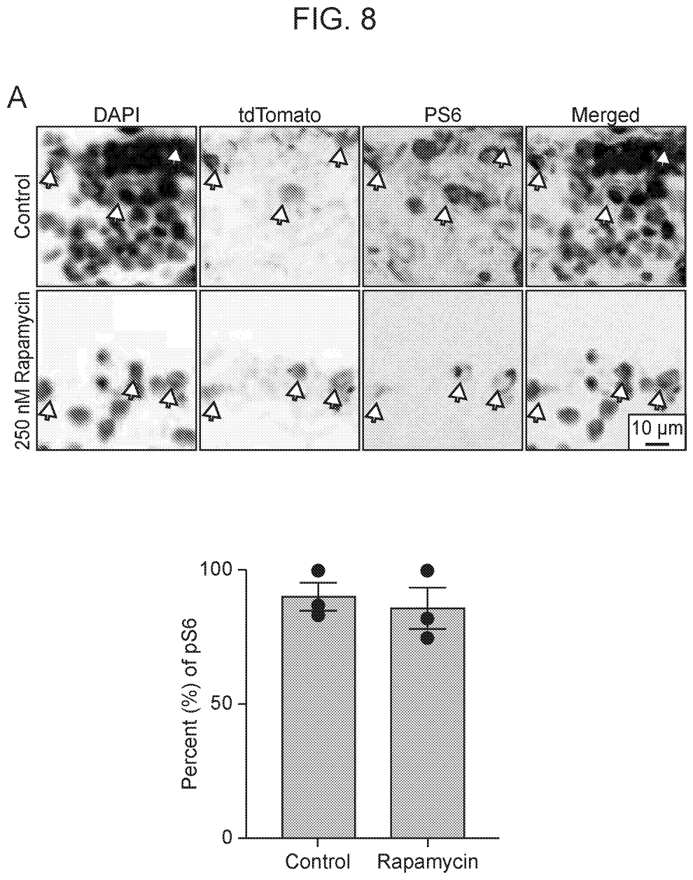

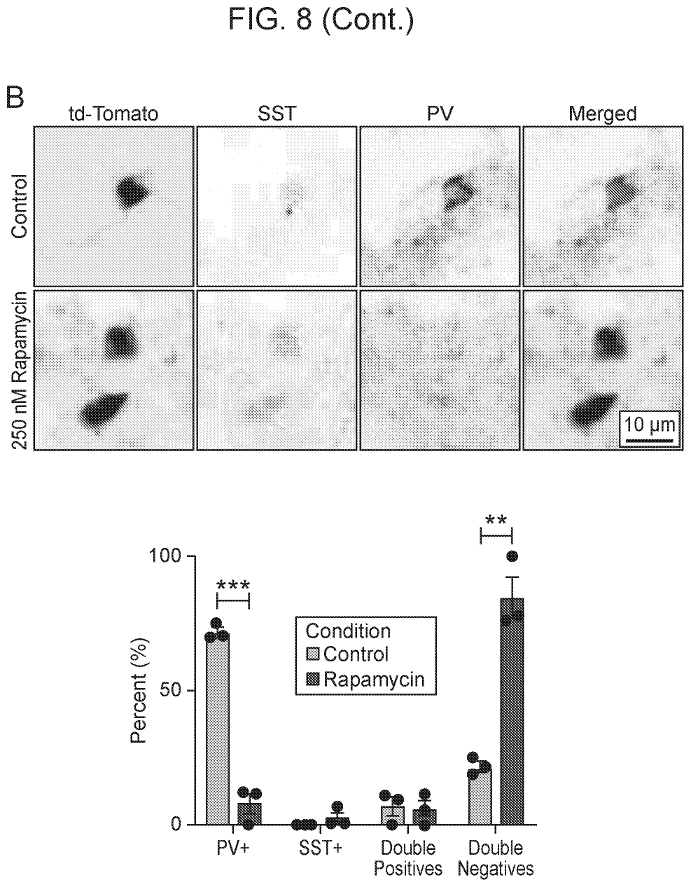

Pivotal work has suggested a role for the mammalian target of Rapamycin (MTOR) signaling pathway in the specification of PV-positive INs (Malik et al., 2019; Wundrach et al., 2020). Specifically, activation of the MTOR pathway via knockout of the upstream MTOR inhibitor TSC1 in MGE-derived INs leads to a modest but higher percentage of PV-INs in the mouse brain (Malik et al., 2019). It was investigated whether treating transplanted organoids chronically with high levels (250 nM) of the MTOR inhibitor rapamycin would lead to a decreased PV expression in the transplanted INs. Organoids with rapamycin since the moment of transplantation for 14 days. As control, vehicle application was performed. MTOR downregulation was assessed by immunostaining against phosphorylated ribosomal protein S6 (pS6), a downstream marker of MTOR activity and of PV fate within the MGE lineage (Malik et al., 2019). It was found that in in chimeric cortical organoids 90.00±8.82% of control treated transplanted INs are positive for this marker 14 DPT, further complementing the findings of PV fate upregulation of transplanted INs (FIGS. 5, 6 and 8A). Rapamycin treatment, however, did not significantly affect pS6 expression of transplanted INs (85.78±12.85% pS6-positive, p>0.05) (FIG. 8A). These results indicate that MGE-derived INs are resistant to rapamycin.

It has previously been shown that the developing human cortex has high levels of MTOR activity in the PN lineage (Andrews et al., 2020; Nowakowski et al., 2017), which is recapitulated in organoid models (Andrews et al., 2020; Pollen et al., 2019). Furthermore, it was observed that in agreement with previous work (Andrews et al., 2020; Pollen et al., 2019) rapamycin treatment ablates pS6 expression in virtually all host cells (FIG. 8A). It is well established that MTOR inhibition in the PN lineage affects the morphology and connectivity of postmitotic excitatory neurons (LiCausi and Hartman, 2018). Having a model in which the transplanted INs are rapamycin resistant while the host cells are rapamycin sensitive allows to test the effect of PN manipulation in the acquisition of PV fate. It was found that 14 DPT 71.66±2.91% of transplanted INs are positive for PV in control organoids (FIG. 8B), in agreement with the finding of progressive acquisition of PV fate upon transplantation (FIG. 5E). On the other hand, only 7.69±6.67% of transplanted INs are immunopositive for PV upon chronic rapamycin treatment (p<0.001), while 84.62±13.34% of transplanted INs are negative for both PV and SST (FIG. 8B). Together, it was shown that PV fate specification is controlled through non cell autonomous mechanisms requiring PNs.

### Reprogramming of Postmitotic SST-positive INS to a PV Fate

In vivo direct lineage reprogramming experiments in mouse cortical PNs identified a progressive loss of fate plasticity through neuronal development and maturation, with a sharp decline in fate plasticity shortly after the PNs become postmitotic (De la Rossa et al., 2013; Rouaux and Arlotta, 2010; Rouaux and Arlotta, 2013; Ye et al., 2015). Yet to date, whether the mechanisms that safeguard cell fate of mammalian neurons are intrinsic and universally applied throughout the central nervous system is unknown.

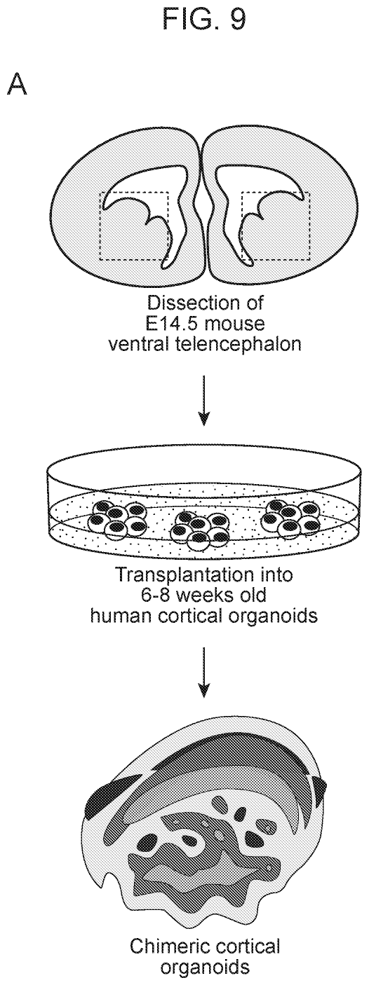

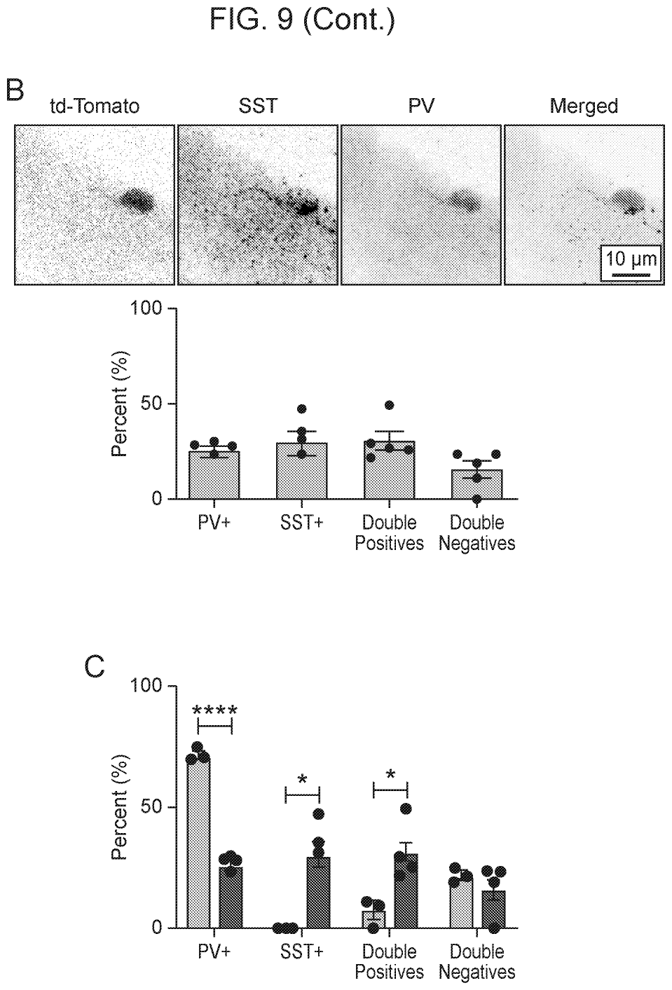

It was investigated whether postmitotic SST-positive INs can be reprogrammed to a PV fate upon transplantation to a human cortical organoid. To accomplish this task, the SST-Cre mouse was used. This mouse line has been heavily characterized in the field. This mouse upregulates the Cre recombinase postmitotically and it is highly specific to SST INs, with minimal leakage to other IN subtypes (Malik et al., 2019; Taniguchi et al., 2011). Breeding this mouse to the Ai14 reporter mouse allows to label postmitotic SST INs during embryonic development (Malik et al., 2019; Taniguchi et al., 2011). The transplantation protocol was modified slightly to further enrich for this population (FIG. 9A): 1). The dissections were performed at E14.5. At this age, the majority of SST-positive INs have been born and are postmitotic (Inan et al., 2012). 2) As postmitotic INs are highly migratory, the entirety of the ventral telencephalon was dissected to account of INs en route to the cortex. Then immunolabeling against the PV and SST reporters was performed at 14 DPT (FIGS. 9B and 9C).

It was found that only 29.28±13.99% of SST-Cre: Ai4 transplanted INs are immunopositive for SST alone (FIG. 9B). On the other hand, over half of transplanted INs were immunopositive of PV. Specifically, while 24.89±6.18% were positive for PV alone, 30.51±10.79% were double positive for PV and SST (FIG. 9B). Given the differences of cell types generated on whether the grafted cells were mitotic or postmitotic at the time of transplantation (FIG. 9C), it was concluded that, as observed in the PN lineage (Rouaux and Arlotta 2010; Rouaux and Arlotta 2013; Ye et al., 2015), INs undergo a progressive restriction of cell fate. Remarkably though, over half of the transplanted INs are capable of switching cell fate at postmitotic stages, unveiling a previously unappreciated potential of cortical INs.

FIG. 8A-8B. A. Representative images and quantifications of phosphorylated ribosomal protein S6 (pS6) in control and rapamycin-treated organoids. n=3 organoids from 2 differentiations for each condition. White arrows=td-Tomato and pS6 positive INs. B. Comparison of INs subtypes obtained in control and rapamycin treated transplanted organoids. **=p<0.01; ***=p<0.001. Error bars represent SEM.

FIG. 9A-9C. A. Experimental design. The entire ventral telencephalon was dissected, dissociated into single cells and transplanted into human brain organoids. B. Representative image and quantification of the fate of transplanted INs into human organoids 14 DPT. n=5 organoids from 2 differentiations. C. Comparison between cell types generated 14 DPT in transplants by either E13.5 NKX2.1-Cre: Ai14 (Differentiation) or SST-Cre: Ai14 (Reprogramming) grafts.

### Experimental Procedures

### Primary Human Cortical Tissue

All primary tissues were obtained and processed as approved by UCSF Gamete, Embryo and Stem Cell Research Committee (GESCR) approval 10-05113. Tissue was collected with patient consent for research and in strict observance of legal and institutional ethical regulations. All samples were de-identified and no sex information is known.

### Mouse Lines and Husbandry

All mouse procedures were previously approved by UCSF IACUC. The mouse lines used for this study have been previously described in literature. Specifically, the Nkx2.1-Cre (Xu et al., 2008), SST-Cre (Taniguchi et al., 2011), Ai14 (Madisen et al., 2010), Ai34 (Daigle et al., 2018) and Ai96 (Madisen et al., 2015) lines were used. Mice were of the C57BL/6J genetic background. For developmental staging, the plug date was considered as day 0.5 (D0.5).

### Induced Pluripotent Stem Cell Maintenance

The iPSC lines 13234 were used, which has been previously described (Pollen et al., 2019). iPSCs were maintained in StemFlex Medium (Thermo Fisher Scientific #A3349401), supplemented with Penicillin/Streptomycin (Thermo Fisher Scientific #15140122). Dissociation and cell passages were done using ReLeSR passaging reagent (Stemcell Technologies #05872) according to manufacturer's instructions.

### Culture of Human and Mouse Organotypic Cortical Sections

Prior to tissue collection fresh artificial cerebrospinal fluid (ACSF) was prepared containing 125 mM sodium chloride (Millipore Sigma #S9888), 2.5 mM potassium chloride (Millipore Sigma #P3911), 1 mM magnesium chloride (Millipore Sigma #M8266), 2 mM calcium chloride (Millipore Sigma #C4901), 1.25 mM sodium phosphate monobasic (Millipore Sigma #71505), 25 mM sodium bicarbonate (Millipore Sigma #S5761), 25 mM D-(+)-glucose (Millipore Sigma #G8270). The solution was bubbled with 95% O₂/5% CO₂.

The tissue was maintained in (ACSF) until embedded in a 3% low melt agarose gel (Millipore Sigma #A9414). Embedded tissue was acute sectioned at 300 μM using a vibratome (Leica) and plated on Millicell inserts (Millipore Sigma #PICM0RG50) in a six well tissue culture plate.

Slices were cultured at the air liquid interface in medium containing 32% Hanks' Balanced Salt solution (Millipore Sigma #H8264), 60% Basal Medium Eagle (Millipore Sigma #B9638), 5% Fetal Bovine Serum (Millipore Sigma F2442), 1% glucose, 1% N2 Supplement and 100 U/mL Penicillin/Streptomycin. Medium was replaced every 2-3 days.

### Human Cortical Organoids Generation

To generate human cortical organoids a variation of our previously described protocol was used (Pollen et al., 2019). iPSCs were dissociated into single cells and re-aggregated in lipidure-coated 96-well V-bottom plates at a density of 10.000 cells per aggregate, in 100 μL of StemFlex Medium supplemented with Rho Kinase Inhibitor (Y-27632, 10 μM, Tocris #1254) (Day −1). After one day (Day 0), the medium was replaced with cortical differentiation medium containing Glasgow Minimum Essential Medium (Thermo Fisher Scientific #11710035), 20% Knockout Serum Replacement (Thermo Fisher Scientific #10828028), 0.1 mM MEM Non-Essential Amino Acids (Thermo Fisher Scientific #11140050), 0.1 mM 2-Mercaptoethanol (Sigma Aldrich #M3148) and 100 U/mL Penicillin/Streptomycin.

Cortical differentiation medium was supplemented with Rho Kinase Inhibitor (Y-27632, 20 μM #days 0-6), WNT inhibitor (IWR1-ε, 3 μM, Cayman Chemical #13659, days 0-18) and TGF-Beta inhibitor (SB431542, Tocris #1614, 5 μM, days 0-18). Media was changed on days 3 and 6 and then every 2-3 days until day 18. On day 18 organoids were transferred to ultra low-adhesion plates and put on an orbital shaker in neuronal differentiation medium at 90 revolutions per minute. Neuronal differentiation medium contained Dulbecco's Modified Eagle Medium: Nutrient Mixture F-12 with GlutaMAX supplement (DMEM/F12 #Thermo Fisher Scientific, 10565018), 1× N-2 Supplement (Thermo Fisher Scientific #17502048), 1× Chemically Defined Lipid Concentrate (Thermo Fisher Scientific #11905031) and 100 U/mL Penicillin/Streptomycin. Organoids were grown under 40% 02 and 5% CO2 conditions. Medium was changed every 2-3 days.

On day 35 onward, 5 μg/mL Heparin sodium salt from porcine intestinal mucosa was added (Sigma Aldrich #H3149) and 1% v/v Matrigel Growth Factor Reduced (GFR) Basement Membrane Matrix, LDEV-free (Matrigel GFR, Corning #354230) to the neuronal differentiation medium.

On day 70 onward, the organoids were transferred to neuronal maturation media containing BrainPhys Neuronal Medium (Stem Cell Technologies #05790), 1× N-2 Supplement, 1× Chemically Defined Lipid Concentrate, 1× B-27 Supplement (Thermo Fisher Scientific #17504044), 100 U/mL Penicillin/Streptomycin and 2% v/v Matrigel Growth Factor Reduced (GFR) Basement Membrane Matrix, LDEV-free.

### Human MGE Organoids Generation

MGE organoids were generated based on a previously published protocol (Birey et al., 2017), albeit with some modifications. iPSCs were dissociated and reaggregated at a density of 10,000 cells per well in lipidure-coated 96-well V-bottom plates. They were reaggregated in Stem Flex medium supplemented with Rho Kinase Inhibitor Y-27632 (Day −1). The next day (day 0), the medium was replaced with Dulbecco's Modified Eagle Medium: Nutrient Mixture F-12 with GlutaMAX supplement, containing 20% (v/v) Knockout Serum Replacement, 1 mM MEM Non-Essential Amino Acids, 0.1 mM 2-Mercaptoethanol and 100 U/mL Penicillin/Streptomycin. Media was supplemented with 5 μM dorsomorphin (Millipore Sigma #P5499), 10 μM TGF-Beta inhibitor SB431542 and 10 μM Rho Kinase Inhibitor Y-27632. Media was changed on days 2 and 4 and did not include Rho Kinase Inhibitor Y-27632.

On Day 6 organoids were transferred to neuronal differentiation media, which contained Neurobasal A medium (ThermoFisher Scientific #10888022), 1× B-27 supplement minus Vitamin A (ThermoFisher Scientific #12587010), 1× GlutaMAX supplement (ThermoFisher Scientific #35050061) and 100 U/mL Penicillin/Streptomycin. Media was changed every 2-3 days. On day 18 organoids were transferred to ultra low-adhesion plates and put on an orbital shaker at 90 revolutions per minute. Neuronal differentiation media was supplemented with small molecules as follows: From day 6 to 11:20 ng/ml Human Recombinant EGF (R&D Systems #236-EG), 20 ng/ml Human Recombinant FGF basic (R&D Systems #233-FB) and 3 μM

WNT inhibitor IWR1-ε. On days 12-15, 100 nM SHH pathway agonist SAG was included (Selleckchem #S7779) and 100 nM retinoic acid (Millipore Sigma #R2625). On days 16-24 retinoic acid was removed from the medium and added 100 nM allopregnanolone (Cayman Chemicals #16930). On day 25 onwards, any small molecules on the medium were not included.

### Human Rhalamic Organoids Generation

Thalamic organoids were generated using a previously described protocol (Xiang et al., 2020). Briefly, iPSCs were clump dissociated into ultralow attachment 6-well plates and cultured for 8 days in Dulbecco's Modified Eagle Medium: Nutrient Mixture F-12 with GlutaMAX supplemented with 15% (v/v) Knockout Serum Replacement, 1 mM MEM Non-Essential Amino Acids, 0.1 mM 2-Mercaptoethanol, 100 U/mL Penicillin/Streptomycin, 10 uM SB431542, 100 nM LDN193189 dihydrochloride (Tocris #6053) and 4 ug/mL insulin (Millipore Sigma #19278). Media was replaced every other day.

Subsequently, plates were moved onto a shaker rocking at 80 rpm, and organoids were cultured for an additional 8 days in a patterning media comprised of Dulbecco's Modified Eagle Medium: Nutrient Mixture F-12 with GlutaMAX supplemented with 15% Dextrose (Millipore Sigma #PHR1000), 0.1 mM 2-Mercaptoethanol, 1% N2 Supplement, 2% B27 Supplement minus Vitamin A, 30 ng/mL recombinant human BMP7 (R&D Systems #354-BP), 1 uM PD325901 (Millipore Sigma #PZ0162) and 100 U/mL Penicillin/Streptomycin, with media replacement every other day.

On day 17 onwards, organoids were cultured in a differentiation media comprised of Neurobasal Medium (ThermoFisher Scientific #21103049) and Dulbecco's Modified Eagle Medium: Nutrient Mixture F-12 with GlutaMAX mixed at a 1:1 ratio, and supplemented with 2% B27 Supplement, 1% N2 Supplement, 0.1 mM MEM Non-Essential Amino Acids, 100 U/mL Penicillin/Streptomycin, 50 uM 2-Mercaptoethanol, and 20 ng/mL BDNF (Millipore Sigma #B3795).

### Mouse Cortical Aggregoids Generation

Mouse E14.5 cortices were dissected and chopped into small pieces. Cortices were then dissociated using Worthington Papain Dissociation System (Worthington #LK003150) according to manufacturer's instructions. Briefly, 20 units of papain per ml, 1 mM L-cysteine and 0.5 mM EDTA were resuspended in Earle's Balanced Salt Solution (EBSS). The enzyme solution was activated by incubating for 30 minutes at 37° C. After activation, 200 units of DNase I per ml were included. The tissue was transferred into the papain and DNase I solution and incubated for 30 minutes at 37° C. shaking at 90 rpm. The tissue was mechanically dissociated using flamed glass Pasteur Pipets (Fisher Scientific #13-678-6B). The tissue was then washed in 1× PBS containing 0.1% Bovine Serum Albumin (Millipore Sigma #A3311) at 300 rpm for 3 minutes. Cells were resuspended and aggregated at a density of 10,000 cells per well in lipidure-coated 96-well V-bottom plates in cortical organoid differentiation medium: Dulbecco's Modified Eagle Medium: Nutrient Mixture F-12 with GlutaMAX supplement, 1× N-2 Supplement, 1× Chemically Defined Lipid Concentrate, 5 μg/mL Heparin sodium salt from porcine intestinal mucosa and 1% v/v Matrigel Growth Factor Reduced (GFR) Basement Membrane Matrix, LDEV-free and 100 U/mL Penicillin/Streptomycin. Organoids were grown under 40% O₂ and 5% CO₂ conditions. Medium was changed every 2-3 days.

### Dissociation and 2D Culture of Primary Human and Mouse Cortical Neurons

Prior to tissue dissociation, glass-bottom cell culture plates (NEST #801006) were coated overnight at 37° C. with 0.1% Poly-L-ornithine solution (Millipore Sigma #P4957). Plates were then washed 3 times with sterile water. Plates were then coated overnight at 37° C. with 5 μg/mL Laminin (Millipore Sigma #L2020) and 1 μg/mL Fibronectin (Millipore Sigma #DLW354008) resuspended in PBS.

Mouse E14.5 cortices and human GW22 cortices were dissected and chopped into small pieces. Cortices were then dissociated using Worthington Papain Dissociation System (for details see Mouse cortical aggregoids generation). Resuspended cells were plated at a concentration of 100,000 cells per well. Cells were grown in cortical organoid differentiation medium: Dulbecco's Modified Eagle Medium: Nutrient Mixture F-12 with GlutaMAX supplement, 1× N-2 Supplement, 1× Chemically Defined Lipid Concentrate, 5 μg/mL Heparin sodium salt from porcine intestinal mucosa and 1% v/v Matrigel Growth Factor Reduced (GFR) Basement Membrane Matrix, LDEV-free and 100 U/mL Penicillin/Streptomycin. Medium was changed every 2-3 days.

### Transplantation of Mouse MGE Interneuron Progenitors

E13.5 MGE were microdissected and transferred to ice cold Leibovitz's L-15 Medium (ThermoFisher Scientific #11415064) supplemented with 180 ug/ml DNase I. The tissue was then mechanically dissociated on ice by pipetting with P1000 micropipette. The dissociated cells were then concentrated by centrifugation (4 min, 800×g).

For human organoids or mouse aggregoids transplantation, the organoids were transferred to individual wells of lipidure-coated 96-well V-bottom plates. A total of 200 ul of fresh cortical organoid neuronal differentiation medium was added: Dulbecco's Modified Eagle Medium: Nutrient Mixture F-12 with GlutaMAX supplement, 1× N-2 Supplement, 1× Chemically Defined Lipid Concentrate, 5 μg/mL Heparin sodium salt from porcine intestinal mucosa and 1% v/v Matrigel Growth Factor Reduced (GFR) Basement Membrane Matrix, LDEV-free and 100 U/mL Penicillin/Streptomycin. Human organoids were 6-8 weeks old at the time of transplantation. Mouse aggregoids had been cultured for 3 weeks before transplantation. A total of 50,000 MGE cells were added into each well and incubated the organoids for 24 hours at 37° C. After this incubation, the organoids were carefully transferred to ultra low attachment tissue culture plates and incubated on an orbital shaker at 90 rpm and 37° C. under 40% O₂ and 5% CO₂ conditions. Medium was changed every 2-3 days.

For transplantation into human and mouse organotypic cultures, the cells were resuspended at a concentration of 1,000 cells/ul. The cell concentrate was then pipetted directly on top of the organotypic cultures and allowed them to integrate.

For coculture with 2D cortical neurons, 10,000 cells were added to each well of the cortical cultures.

### MTOR Inhibition

Transplanted organoids were grown as described. 250 nM rapamycin (Millipore Sigma #R8781) was added in the media from the transplantation throughout the duration of the experiment. Control organoids were treated with vehicle media.

### Immunohistochemistry and Confocal Imaging

Organoids were collected and fixed in 4% Paraformaldehyde (PFA) (ThermoFisher Scientific #28908) and cryopreserved in 30% Sucrose (Millipore Sigma #S8501). They were then embedded in a solution containing 50% of Tissue-Tek O.C.T. Compound (Sakura #4583) and 50% of 30% sucrose dissolved in in 1× Phosphate-buffered saline (PBS) pH 7.4 (Thermo Fisher #70011044). They were then sectioned to 12 μm using a cryostat (Leica Biosystems #CM3050) directly onto glass slides. After 3 washes of 10 minutes in 1× PBS, the sections were incubated in blocking solution 5% donkey serum, 2% gelatin and 0.1% Triton X-100 for 1 hour. The sections were then incubated in primary antibodies overnight at 4° C. They were then washed 3 times for 30 minutes and incubated in secondary antibodies for 90 minutes at room temperature. They were then washed 3 times for 30 minutes in PBS and one time in sterile water for 10 minutes.

Whole human and mouse organotypic sections were fixed with 4% PFA for 2 hours at room temperature and then washed in PBS at 4° C. overnight. Blocking was done for one day at 4° C. Primary antibody incubation was done for 3 days at 4° C. followed by 3 2 hours PBS washes. Secondary antibody incubation was done for 3 days at 4° C. followed by 3-2 hours PBS washes and 1 30 min wash with sterile water.

Primary antibodies used were: rabbit anti BDNF (Abcam #ab108319, 1:100); rat anti CTIP2 (Abcam #ab18465, 1:100); mouse anti ERBB4 (ThermoFisher #MA5-12888; 1:100); mouse anti HNA (Sigma Aldrich #MAB1281; 1:100); rabbit anti MAP2 (Proteintech #17490-1-AP. 1:100), rabbit anti MEF2C (Abcam #ab227085, 1:250); mouse anti NR2F2 (Novus Biologicals #PP-H7147-00, 1:100); rabbit anti PV (Swant #PV27; 1:250); rabbit anti PSD95 (ThermoFisher #51-6900, 1:100); chicken anti RFP (Rockland #600-901-379, 1:100); rabbit anti S6 phosphorylated (pS6) (S235/236) (Cell Signaling #2211, 1:100); mouse anti SST (Santa Cruz Biotechnology #sc-55565, 1:100). Secondary antibodies were of the Alexa series (ThermoFisher), used at a concentration of 1:250. In addition, biotin-conjugated WFA (Vector laboratories #B-1355-2, 1:200) were used, which was visualized using Alexa 488-conjugated Streptavidin (Thermo Fisher #S11223; 1:500).

Of note, only the BDNF antibody required antigen retrieval, which was done by incubating the slides at 95° C. for 20 minutes in “Antigen retrieval solution” containing 10 mM Trisodium citrate dihydrate (Millipore Sigma #S1804) and 0.05% Tween 20 (Millipore Sigma #P1379) at pH 6.0 before blocking.

Imaging was done using an inverted confocal microscope (Leica CTR 6500) and LAS AF software (Leica). Images were processed using ImageJ software (NIH). Overlays and quantifications were done using Adobe Photoshop version 2020 (Adobe).

### Whole Organoid Immunostaining, Clearing and Light Sheet Imaging

Human brain organoids were fixed at room temperature for 45 minutes in 4% paraformaldehyde. After fixation, they were washed 3 times in PBS and stored at 4° C. For whole-organoid immunostaining and tissue clearing, the organoids were blocked for 24 hours at room temperature in PBS supplemented with 0.2% gelatin (VWR, 24350.262) and 0.5% Triton X-100 (Millipore Sigma, X100) (PBSGT). Samples were then incubated with primary antibodies for 7 days at 37° C. at 70 rpm in PBSGT+1 mg/ml Saponin Quillaja sp (Sigma Aldrich, S4521) (PBSGTS).

Following primary antibody incubation, samples were washed 6 times in PBSGT over the course of one day at room temperature. For nuclear staining Syto16 green fluorescent dye was used (ThermoFisher Scientific, S7578). Secondary and nuclear staining was performed at for 1 day at 37° C. at 70 rpm in PBSGTS. Samples were then washed 6 times in PBSGT over the course of one day at room temperature.

Whole organoid clearing was performed using ScaleCUBIC-1 solution as described in (Suzaki et al., 2015). Briefly, the solution contained: 25% wt Urea (Millipore Sigma, U5378), 25% wt N,N,N′,N′-Tetrakis (2-hydroxypropyl) ethylenediamine (Tokyo Chemical Industry, T0781) and 15% Triton X-100 dissolved in distilled water. Organoids were incubated in ScaleCUBIC-1 solution overnight at room temperature at 90 rpm. Whole organoid imaging was performed using a custom made Lattice Light Sheet Microscope (UCSF Biological Imaging Development Center) and the images were deconvoluted using Richardson-Lucy algorithm. Images were then processed using Imaris 9.2 software (Bitplane).

### Statistical Analysis of Immunohistochemistry Quantifications

All statistical analysis was performed using Prism 8.4.3 (GraphPad Software). Conditions were compared using unpaired parametric Student's t-test without Welch's correction.

### Maximal Soma Size Measurements

In order to avoid any potential bias in image acquisition, images obtained for marker quantifications were re-used. Random sections were used. Files were processed using ImageJ 2.0.0-rc-54/1.51h. The Z Project function was first used to create a single plane image of the z-stacks using Maximal Intensity as the projection type parameter. The freehand selection tool was then used to delineate the area of the maximal soma size. Finally, the Measure tool to calculate the area of the maximal soma.

### Live Imaging of Organoids

Live imaging was performed as previously described (Huang et al., 2020). 1 DPT, the transplanted organoids were moved to Millicell inserts (Millipore Sigma #PICMORG50) on a six well glass-bottom tissue culture plate for culture in air liquid interface. The medium used for culture was cortical organoid neuronal differentiation medium without Matrigel. Organoids were then incubated for 6 hours at 37° C. prior to imaging to allow for tissue flattening.

Imaging was performed on an inverted Leica TCS SP5 confocal microscope with an on-stage incubator streaming 5% CO₂, 8% O₂, and balanced N₂ into the chamber. The chamber temperature was 37° C. Slices were imaged for 24 hours using a 10× air objective (Zoom 2×) at 20 min intervals.

### Calcium Imaging of Organoids

Calcium imaging was done using genetically encoded calcium indicators. The imaging was performed in an inverted confocal microscope (Leica CTR 6500) using the LAS AF software (Leica). Images were taken every 1.2 seconds and processed using ImageJ software.

### REFERENCES

Ali F, Kwan A C. (2019). Interpreting in vivo calcium signals from neuronal cell bodies, axons, and dendrites: a review. Neurophotonics 7 (1): 011402. 10.1117/1.NPh.7.1.011402

Andrews M G, Subramanian L, Kriegstein A R. (2020). mTOR signaling regulates the morphology and migration of outer radial glia in developing human cortex. eLife 9: e58737. DOI: 10.7554/eLife.58737

Bhaduri A, Di Lullo E, Jung D, Müller S, Crouch E E, Espinosa C S, et al. (2020). Outer Radial Glia-like Cancer Stem Cells Contribute to Heterogeneity of Glioblastoma. Cell Stem Cell 26 (1): 48-63. DOI: 10.1016/j.stem.2019.11.015

Birey F, Andersen J, Makinson C D, Islam S, Wei W, Huber N, et al. (2017). Assembly of functionally integrated human forebrain spheroids. Nature 545:54-59. DOI: 10.1038/nature22330.

Caillard O, Moreno H, Schwaller B, Llano I, Celio M R, Marty A. (2000). Role of the calcium-binding protein parvalbumin in short-term synaptic plasticity. PNAS 97 (24): 13372-13377. DOI: 10.1073/pnas.230362997.

Carceller H, Guirado R, Ripolles-Campos E, Teruel-Marti V, Nacher J. (2020). Perineuronal nets regulate the inhibitory perisomatic input onto parvalbumin interneurons and gamma activity in the prefrontal cortex. Journal of Neuroscience. DOI: 10.1523/JNEUROSCI.0291-20.2020

Chen Y J J, Friedman B A, Ha C, Durinck S, Liu J, Rubenstein J L, et al. (2017). Single-cell RNA sequencing identifies distinct mouse medial ganglionic eminence cell types. Scientific Reports 7:45656. DOI: 10.1038/srep45656

Daigle T L, Madisen L, Hage T A, Valley M T, Knoblich U, Larsen R S, et al. (2018). A Suite of Transgenic Driver and Reporter Mouse Lines with Enhanced Brain-Cell-Type Targeting and Functionality. Cell 174 (2): 465-480. DOI: 10.1016/j.cell.2018.06.035

De la Rossa A, Bellone C, Golding B, Vitali I, Moss J, Toni N, et al. (2013). In vivo reprogramming of circuit connectivity in postmitotic neocortical neurons. Nature Neuroscience 16 (2): 193-200. DOI: 10.1038/nn.3299.

Giandomenico S L, Sutcliffe M, and Lancaster M A. (2021). Generation and long-term culture of advanced cerebral organoids for studying later stages of neural development. Nature Protocols 16:579-602. DOI: 10.1038/s41596-020-00433-w.

Hsich J Y, Baraban S C. (2017). Medial Ganglionic Eminence Progenitors Transplanted into Hippocampus Integrate in a Functional and Subtype-Appropriate Manner. cNeuro 4 (2): ENEURO.0359-16.2017. DOI: 10.1523/ENEURO.0359-16.2017.

Hu J S, Vogt D. Lindtner S, Sandberg M, Silberberg S N, Rubenstein J L R. (2017). Coup-TF1 and Coup-TF2 control subtype and laminar identity of MGE-derived neocortical interneurons. Development 144: 2837-2851. DOI: 10.1242/dev. 150664.

Huang W, Bhaduri A, Velmeshev D. Wang S, Wang L, Rottkamp C A, et al. (2020). Origins and Proliferative States of Human Oligodendrocyte Precursor Cells. Cell 182 (3): 594-608.e11. DOI: 10.1016/j.cell.2020.06.027.

Huang Z J, Kirkwood A, Pizzorusso T, Porciatti V, Morales B, Bear M F, et al. (1999). BDNF Regulates the Maturation of Inhibition and the Critical Period of Plasticity in Mouse Visual Cortex. Cell 98 (6): 739-755. DOI: 10.1016/S0092-8674(00)81509-3.

Inan M, Walagen J, and Anderson S A. (2012). Spatial and Temporal Bias in the Mitotic Origins of Somatostatin- and Parvalbumin-Expressing Interneuron Subgroups and the Chandelier Subtype in the Medial Ganglionic Eminence. Cerebral Cortex 22 (4): 820-827. DOI: 10.1093/cercor/bhr148.

Jager P, Moore G, Calpin P, Durmishi X, Salgarella I, Menage L, et al. (2020). Dual midbrain and forebrain origins of thalamic inhibitory interneurons. eLife 10: e59272. DOI: 10.7554/eLife.59272.

Kooijmans R S, Sierhuis W, Self M W, Roelfsema P R. (2020). A Quantitative Comparison of Inhibitory Interneuron Size and Distribution between Mouse and Macaque V1, Using Calcium-Binding Proteins. Cerebral Cortex Communications 1: tgaa068. DOI: 10.1093/texcom/tgaa068.

LiCausi F, Hartman N W. (2018). Role of mTOR Complexes in Neurogenesis. International Journal of Molecular Sciences 19 (5): 1544. DOI: 10.3390/ijms19051544.

Lodato S, Rouaux C, Quast K B, Jantrachotechatchawan C, Studer M, Hensch T K. Arlotta P. (2011). Excitatory projection neuron subtypes control the distribution of local inhibitory interneurons in the cerebral cortex. Neuron 69 (4): 763-779. DOI: 10.1016/j.neuron.2011.01.015

Madisen L, Zwingman T A, Sunkin S M, Oh S W, Zariwala H A, Gu H, et al. (2010). A robust and high-throughput Cre reporting and characterization system for the whole mouse brain. Nature Neuroscience 13 (1): 133-40. DOI: 10.1038/nn.2467.

Madisen L, Garner A R, Shimaoka D, Chuong A S, Klapoetke N C, Li L, et al. (2015). Transgenic mice for intersectional targeting of neural sensors and effectors with high specificity and performance. Neuron 85:942-958. DOI: 10.1016/j.neuron.2015.02.022.

Malik R, Pai E L, Rubin A N, Stafford A M, Angara K, Minasi P, et al. (2019). Tsc1 represses parvalbumin expression and fast-spiking properties in somatostatin lineage cortical interneurons. Nature Communications 10:4994. DOI: 10.1038/s41467-019-12962-4.

Mayer C, Hafemeister C, Bandler R C, Machold R, Brito R B, Jaglin X, et al. (2018). Developmental diversification of cortical inhibitory interneurons. Nature 555: 457-462. DOI: 10.1038/nature25999.

Mostajo-Radji M A, Pollen A A. (2018). Postmitotic Fate Refinement in the Subplate. Cell Stem Cell 23 (1): 7-9. DOI: 10.1016/j.stem.2018.06.017.

Mostajo-Radji M A, Schmitz M T, Montoya S T, Pollen A A. (2020). Reverse engineering human brain evolution using organoid models. Brain Research 1729:146582.

Nowakowski T J, Bhaduri A, Pollen A A, Alvarado B, Mostajo-Radji M A, Di Lullo E, et al. (2017). Spatiotemporal gene expression trajectories reveal developmental hierarchies of the human cortex. Science 358 (6368): 1318-1323. DOI: 10.1126/science.aap8809

Pham M T, Pollock K M, Rose M D, Cary W A, Stewart H R, Zhou P, et al. (2018). Generation of human vascularized brain organoids. Neuroreport 29 (7): 588-593. DOI: 10.1097/WNR.0000000000001014

Pollen A A, Bhaduri A, Andrews M G, Nowakowski T J, Meyerson O S, Mostajo-Radji M A, et al. (2019). Establishing Cerebral Organoids as Models of Human-Specific Brain Evolution. Cell 176 (4): 743-756. DOI: 10.1016/j.cell.2019.01.017.

Quadrato G, Nguyen T, Macosko E Z, Sherwood J L, Yang S M, Berger D R, et al. (2017). Cell diversity and network dynamics in photosensitive human brain organoids. Nature 545: 48-53. DOI: 10.1038/nature22047.

Real R, Peter M, Trabalza A, Khan S, Smith M A, Dopp J, et al. (2018). In vivo modeling of human neuron dynamics and Down syndrome. Science 362 (6416): eaau1810. DOI: 10.1126/science.aau1810.

Rouaux C, Arlotta P. (2010). Fezf2 directs the differentiation of corticofugal neurons from striatal progenitors in vivo. Nature Neuroscience 13:1345-1347. DOI: 10.1038/nn.2658.

Rouaux C, Arlotta P. (2013). Direct lineage reprogramming of post-mitotic callosal neurons into corticofugal neurons in vivo. Nature Cell Biology 15 (2): 214-221. DOI: 10.1038/ncb2660.

Schmunk G, Kim C N, Soliman S S, Keefe M G, Bogdanoff D, Tejera D, et al. (2020). Human microglia upregulate cytokine signatures and accelerate maturation of neural networks. Biorxiv. DOI: 10.1101/2020.03.24.006874.

Shmakov S, Smargon A, Scott D, Cox D, Pyzocha N, Yan W, et al. (2017). Diversity and evolution of class 2 CRISPR-Cas systems. Nature Reviews Microbiology 15(3): 169-182. DOI: 10.1038/nrmicro.2016.184.

Su J, Cole J. Fox M A. (2017). Loss of Interneuron-Derived Collagen XIX Leads to a Reduction in Perineuronal Nets in the Mammalian Telencephalon. ASN Neuro 9 (1): 1759091416689020. DOI: 10.1177/1759091416689020.

Sun Y, Ikrar T, Davis M F, Gong N, Zheng X, Luo Z D. (2016). Neuregulin-1/ErbB4 Signaling Regulates Visual Cortical Plasticity. Neuron 92 (1): 160-173. DOI: 10.1016/j.neuron.2016.08.033.

Taniguchi H, He M, Wu P, Kim S, Paik R, Sugino K, et al. (2011). A Resource of Cre Driver Lines for Genetic Targeting of GABAergic Neurons in Cerebral Cortex. Neuron 71 (6): 995-1013. DOI: 10.1016/j.neuron.2011.07.026.

Tomas F J B, Turko P, Heilmann H, Trimbuch T, Yanagawa Y, Vida I, et al. (2020). BDNF Expression in Cortical GABAergic Interneurons. International Journal of Molecular Sciences 21 (5): 1567. DOI: 10.3390/ijms21051567

Wen T H, Binder D K, Ethell I M, Razak K A. (2018). The Perineuronal ‘Safety’ Net? Perineuronal Net Abnormalities in Neurological Disorders. Frontiers in Molecular Neuroscience: 00270. DOI: 10.3389/fnmol.2018.00270

Wundrach D, Martinetti L E, Stafford A M, Bilinovich S M, Angara K, Prokop J W, et al. (2020). A human TSC1 variant screening platform in GABAergic cortical interneurons for genotype to phenotype assessments. Frontiers in Molecular Neuroscience. 13:573409. DOI: 10.3389/fnmol.2020.573409.

Xiang Y, Cakir B, Park I. (2020). Generation of Regionally Specified Human Brain Organoids Resembling Thalamus Development. STAR Protocols 1 (1): 100001. DOI: 10.1016/j.xpro.2019.100001.

Xu Q, Tam M, Anderson S A. (2008). Fate mapping Nkx2.1-lineage cells in the mouse telencephalon. The Journal of Comparative Neurology 506 (1): 16-29. DOI: 10.1002/cne.21529.

Ye Z, Mostajo-Radji M A, Brown R, Rouaux C, Tomassy G S, Hensch T K, Arlotta P. (2015). Instructing Perisomatic Inhibition by Direct Lineage Reprogramming of Neocortical Projection Neurons. Neuron 88 (3): 475-483. DOI: 10.1016/j.neuron.2015.10.006

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method for generating an enriched population of parvalbumin-positive interneurons, the method comprising culturing a population of mouse primary neuronal progenitors in a human brain organoid or a primary brain slice of human origin, wherein the mouse primary neuronal progenitors differentiate into parvalbumin-positive interneurons in the brain organoid or the primary brain slice, thereby generating an enriched population of parvalbumin-positive interneurons.

2. The method of claim 1, further comprising determining the number of parvalbumin-positive interneurons in the brain organoid or the primary brain slice.

3. The method of claim 1, wherein the enriched population of parvalbumin-positive interneurons is generated within 2 days of said culturing.

4. The method of claim 1, wherein at least 50% of the population of primary neuronal progenitors differentiate into parvalbumin-positive interneurons.

5. The method of claim 1, wherein the mouse primary neuronal progenitors are medial ganglionic eminence (MGE) neuronal progenitors or post-mitotic somatostatin-positive interneurons.

6. The method of claim 5, wherein the MGE neuronal progenitors are genetically modified to reduce expression of or to render a target gene non-functional.

7. The method of claim 6, wherein the target gene is selected from PVALB, SST, MEF2C, GAD2, DLX5, DLX6, NKX2-1, MTOR, TSC1, TSC2, MECP2, PTEN, RYK, CHD8, ERBB4, MAKA, SCN1A, EGFR, SYT2, GLI2, and LHX6.

8. The method of claim 5, wherein the MGE neuronal progenitors are genetically modified with a heterologous nucleic acid comprising a nucleotide sequence encoding a heterologous gene product of interest.

9. The method of claim 5, wherein the heterologous gene product is a polypeptide selected from PVALB, SST, MEF2C, GAD2, DLX5, DLX6, NKX2-1, MTOR, TSC1, TSC2, MECP2, PTEN, RYK, CHD8, ERBB4, MAKA, SCN1A, EGFR, SYT2, GLI2, and LHX6, and where the polypeptide comprises one or more mutations compared to wild-type.

10. The method of claim 1, wherein the enriched population comprises from about 10⁴ to about 10⁷ parvalbumin-positive interneurons.

11. The method of claim 10, wherein at least 80% of the enriched population are parvalbumin-positive interneurons.

12. The method of claim 1, further comprising:
a) contacting the population of parvalbumin-positive interneurons with a test agent; and
b) determining the effect of the test agent on a feature of the parvalbumin-positive interneurons.

13. The method of claim 12, wherein the feature is viability, physiology, morphology, connectivity, or gene expression.

14. The method of claim 12, wherein the feature is expression of a gene product, wherein the gene product is an mRNA or a polypeptide encoded by a gene selected from PVALB, SST, MEF2C, GAD2, DLX5, DLX6, NKX2-1, MTOR, TSC1, TSC2, MECP2, PTEN, RYK, CHD8, ERBB4, MAKA, SCN1A, EGFR, SYT2, GLI2, and LHX6.

15. A chimeric organoid comprising:
a) human brain organoid; and
b) an enriched population of mouse parvalbumin-positive interneurons.

16. The chimeric organoid of claim 15, wherein the enriched population comprises from about 10³ to about 10⁷ parvalbumin-positive interneurons.

17. The chimeric organoid of claim 15, wherein at least 80% of the enriched population are parvalbumin-positive interneurons.

18. The chimeric organoid of claim 15, wherein the mouse PV⁺ interneurons are within a perineuronal net.

19. The chimeric organoid of claim 15, wherein the chimeric organoid is a chimeric cortical organoid.

20. A method of identifying an agent that enhances function of a parvalbumin-positive interneuron, the method comprising:
a) contacting the chimeric organoid of claim 1 with a test agent; and
b) determining the effect of the test agent on a feature of the parvalbumin-positive interneurons, wherein a test agent that enhances the feature is a candidate agent for enhancing the function of the parvalbumin-positive interneuron.

21-22. (canceled)
